(19) United States
(12) Patent Application Publication
(10) Pub. No.: US 2015/0160186 A1
(43) Pub. Date: Jun. 11, 2015

(54) Diabetic Testing Supply Dispenser

(71) Applicant: Steven Garner-Richards, Homosassa, FL (US)

(72) Inventor: Steven Garner-Richards, Homosassa, FL (US)

(21) Appl. No.: 14/266,060

(22) Filed: Apr. 30, 2014

Related U.S. Application Data
(60) Provisional application No. 61/913,605, filed on Dec. 9, 2013.

(51) Int. Cl. G01N 33/487

(52) U.S. Cl. CPC G01N 33/48757; G01N 33/48778

(57) ABSTRACT

Single-use and refillable diabetic testing supply dispensers are disclosed. In various embodiments, the dispenser uses an ejector coupled to an actuator external to the dispenser to move single testing supplies contained within the internal volume of the dispenser through a port in the dispenser for selection by the user. In various embodiments the dispenser has a dispenser head unit that is coupled to one of a variety of magazine designs which store the testing supplies to be dispensed. A bulk loader rapidly refills the dispenser. In some embodiments, testing supplies are affixed to a carrier strip which is carried on a spool internal to the dispenser, incremented out of the dispenser, and cut to dispense a single testing supply during each operation. In some embodiments, tamper resistant elements are added to discourage fraudulent resale of the testing supplies contained in the dispenser. In some embodiments, the dispenser includes a lancing device that is fed by a magazine and deters re-use of lancets.

### REFERENCE TO RELATED APPLICATIONS

This application claims one or more inventions which were disclosed in Provisional Application No. 61/913,605, filed Dec. 9, 2013, entitled “Diabetic Testing Supply Dispenser”. The benefit under 35 USC §119(e) of the United States provisional application is hereby claimed, and the aforementioned application is hereby incorporated herein by reference.

### BACKGROUND OF THE INVENTION

1. Field of the Invention

The invention pertains to the field of diabetic testing supplies. More particularly, the invention pertains to dispensers for blood glucose test strips and lancets.

2. Description of Related Art

Patients suffering from diabetes are required to perform blood glucose tests on their own blood at regular intervals to ensure that they take oral medication or self-inject with insulin at appropriate times and in proper doses. While specific testing protocols vary, in general they require the patient to draw a small quantity of blood by piercing their skin with a lancet. One end of a disposable, single-use, test strip is then inserted into a blood glucose meter. Having prepared the meter for testing, the free end of the test strip is brought into contact with a drop of the patient's blood drawn with the lancet. Blood is drawn into the test strip, and the glucose meter displays a value correlated to the blood glucose level for that sample.

According to the American Diabetes Association, insulin dependent diabetics should test their blood four to seven times per day. Further, testing protocol dictates that a fresh, sterile lancet be used for each test for reasons of sterility. As a result, these patients require a large supply of disposable testing strips and lancets to comply with recommended testing protocols. Test strips are therefore sold in bulk quantities, and a single package may contain between 25 and 100 strips. Packaging of the strips is often performed by simply placing a quantity of the strips in a small canister with a press on lid.

Lancets have historically been constructed as flat strips of surgical steel with a double edge blade at one end. More recently, single use, disposable, needle lancets have been developed in conjunction with lancing devices that allow the patient to customize the lancet penetration depth. Needle lancets are made available, as with test strips, in bulk quantities (e.g., 100 lancets per package) in conventional product packaging.

However, these packaging methods have several issues associated with them. Individual test strips are relatively small; for example, approximately 30 mm×5 mm×1 mm. Loose packing of large numbers of strips in a canister makes it difficult for some patients to isolate and grasp an individual strip from the bulk package, particularly the elderly. As a result, strips may become lost, damaged, dropped and adulterated, or otherwise unusable. Bulk packaging also requires vigilance on the part of the patient to take care when opening the canister, and closing the canister after selecting a strip, to prevent contamination of the remaining strips or spillage of the container, which may also result in losing test strips or damaging them. Opening and closing test strip containers can also be an issue for some patients with compromised motor skills.

Furthermore, while the canisters currently used to package bulk quantities of test strips are individually relatively inexpensive to manufacture, the sheer quantity of strips used in a large population results in manufacturers making significant expenditures in packaging to supply this demand.

The cost of diabetic testing supplies is often covered by government subsidized healthcare reimbursement plans, such as Medicaid insurance programs in the United States. These costs, for test strips alone, can be significant. A study published by the Office of the New York State Comptroller, Division of State Government Accountability (Report 2008-S-123), and herein incorporated by reference, investigated the costs incurred by Medicaid in particular. The study determined that for a five year period ending in 2008, Medicaid programs within the State of New York had spent $254 million on test strips alone.

More importantly, the study pointed out numerous inconsistencies in test strip and lancet insurance claim patterns. These inconsistencies include: patients ordering more test strips than lancets; and patients ordering test strips but no lancets. Further, the study showed that the New York Medicaid program generally provided more test strips to Medicaid beneficiaries in New York State than use patterns showed were provided in similar programs in other demographic areas. Even more significantly, the study showed that in many cases patients had acquired, through government subsidized health insurance, far more test strips than they could possibly use. Based on these data alone, it is clear that the one-to-one recommended ratio of test strips to lancets, i.e. using a fresh lancet with each blood test, is not followed by many patients.

In a simple analysis of this study, one can also conclude that significant waste of healthcare funds occurs through simple mismanagement of test strip and lancet ordering, delivery, and dispensing procedures. A more detailed analysis, however, shows that an excess supply of test strips has led to the growth of a “gray market”, with patients offering their excess test strips for sale on the internet to patients who would otherwise pay out-of-pocket from legitimate sources. Test strips obtained intentionally through fraudulent insurance claims may also similarly flow into this gray market.

As strips are packaged in bulk and therefore relatively untraceable after sale, test strip aggregators have developed that purchase excess strips from patients, re-package them in quantity, and offer them for resale. This practice is undesirable on at least two grounds. First, resale of test strips that have been either legitimately or fraudulently obtained with public funds (e.g., government subsidized insurance claims) results in an unnecessary, and fraudulent, financial burden on those systems. Secondly, as the excess test strips have in many cases been in opened packaging, or even worse repackaged, their integrity cannot be assured. As a result, the gray market contributes to an illegal trade that may also result in delivery of adulterated medical supplies to otherwise unsuspecting patients.

Similar issues also relate to the bulk packaging of lancets. The temptation for resale of lancets, whether excess lancets acquired legitimately or through fraudulent means, is less than that for test strips. However, the handling of bulk quantities of lancets remains cumbersome, and temptation to re-use lancets contrary to sterile procedure is high.

### SUMMARY OF THE INVENTION

Dispensers for diabetic testing supplies include single-use dispenser embodiments and refillable dispenser embodiments. In various embodiments, the dispenser uses an ejector coupled to an actuator external to the dispenser to move single testing supplies contained within the internal volume of the dispenser through a port in the dispenser for selection by the user. These embodiments include, but are not limited to, linear slides with ejectors attached to them, rotating drum ejectors, and cylinders containing individual testing supplies that are rotated into alignment with a dispenser port. In various embodiments, the dispenser has a dispenser head unit that is coupled to one of a variety of magazine designs which store the testing supplies to be dispensed. Some magazine embodiments include, but are not limited to, linear feed magazines, staggered stack magazines, serpentine magazines, and circular, spiral, or helical feed magazines. A bulk loader rapidly refills the dispenser. In some embodiments, testing supplies are affixed to a carrier strip which is carried on a spool internal to the dispenser, incremented out of the dispenser, and cut to dispense a single testing supply during each operation. In some embodiments, tamper resistant elements are added to discourage fraudulent resale of the testing supplies contained in the dispenser. Tamper resistant elements include, but are not limited to, fluid containing ampules that break when attempts to open the dispenser are made, and applicators that apply a marking fluid to each testing supply as it is dispensed. In some embodiments, the dispenser includes a lancing device that is fed by a magazine and deters re-use of lancets.

### BRIEF DESCRIPTION OF THE DRAWINGS

FIG. 1 shows a frontal perspective of a testing supply dispenser for test strips.

FIG. 2 shows a longitudinal cross-section of the dispenser of FIG. 1 and its internal elements.

FIG. 3 shows a longitudinal cross-section of the dispenser of FIG. 1 in operation.

FIG. 4 shows a longitudinal cross-section of the dispenser of FIG. 1 in operation with a test strip in the dispensed position.

FIG. 5 shows a longitudinal cross-section of the dispenser of FIG. 1 with a slide return spring.

FIG. 6 shows a longitudinal cross-section of the dispenser of FIG. 1 with a marking fluid reservoir and applicator.

FIG. 7 shows a frontal perspective of a testing supply dispenser for lancets.

FIG. 8 shows a longitudinal cross-section of the dispenser of FIG. 7 and its internal elements including a return spring, a marking fluid reservoir, and an applicator.

FIG. 9 shows a lateral cross-section of the dispenser of FIG. 7 including feed regulators.

FIG. 10 shows a detailed lateral cross-section of the feed regulators of FIG. 9.

FIG. 11 shows a longitudinal cross-sectional detail of the feed regulators of FIG. 9 in relation to an ejector and two lancets.

FIG. 12 shows a lateral cross-section of the dispenser of FIG. 7 having a stacked magazine storage volume.

FIG. 13 shows a lateral cross-section of the dispenser of FIG. 7 having a stacked magazine storage volume after dispensing of all lancets.

FIG. 14 shows a lateral cross-section of the dispenser of FIG. 7 having a serpentine magazine storage volume.

FIG. 15 shows a lateral cross-section of the dispenser of FIG. 7 having a circular magazine storage volume filled with lancets.

FIG. 16 shows a lateral cross-section of the dispenser of FIG. 7 having a circular magazine storage volume filled with test strips.

FIG. 17A shows frontal perspective of a rectalinear fracturable ampule containing a fluid.

FIG. 17B shows frontal perspective of an arcuate fracturable ampule containing a fluid.

FIG. 17C shows frontal perspective of a trapezoidal fracturable ampule containing a fluid.

FIG. 18 shows a longitudinal cross-section of the dispenser of FIG. 5 having fracturable ampules containing fluid integrated into the walls of the dispenser.

FIG. 19 shows a lower perspective of a slide mechanism having a fracturable ampule containing fluid integrated into the slide.

FIG. 20 shows a longitudinal cross-section of a slide mechanism having a fracturable ampule containing fluid integrated into the slide.

FIG. 21 shows a longitudinal cross-section of a dispenser of having fracturable ampules containing fluid integrated into the walls and bottom of the dispenser.

FIG. 22 shows a lateral cross-section of a dispenser having a separable magazine group and dispenser head group.

FIG. 23 shows a lateral cross-section of the dispenser head group and magazine group of FIG. 22 separated.

FIG. 24 shows the dispenser of FIG. 14 having a separable magazine group and dispenser head group.

FIG. 25 shows the dispenser of FIG. 16 having a separable magazine group and dispenser head group.

FIG. 26 shows the dispenser of FIG. 12 having a separable magazine group and dispenser head group.

FIG. 27 shows a stack of test strips bound by a removable band.

FIG. 28 shows a dispenser having magazine volume sears.

FIG. 29 shows a lateral cross-section of a magazine group having magazine volume sears and head group sear blocks.

FIG. 30 shows a bound stack of test strips being bulk loaded into a magazine group.

FIG. 31 shows a bound stack of test strips being held in a magazine by sears.

FIG. 32 shows the release of magazine sears by connection of a head group.

FIG. 33 shows a top frontal perspective of a dispenser with a slot allowing passage of a leader attached to a band surrounding a stack of test strips.

FIG. 34 shows a dispenser bulk loader for testing supplies.

FIG. 35 shows a dispenser bulk loader for testing supplies filled with test strips.

FIG. 36 shows a dispenser bulk loader for testing supplies filled with lancets.

FIG. 37 shows a lateral cross-section of a dispenser bulk loader filled with lancets inserted into a magazine group.

FIG. 38 shows a lateral cross-section of a dispenser bulk loader inserted into a magazine group loading lancets.

FIG. 39 shows a lateral cross-section of a dispenser bulk loader inserted into a magazine group when loading lancets is completed.

FIG. 40 shows a horizontal cross-section of a magazine group having feed regulators and a feed stop mechanism.

FIG. 41 shows a magazine group filled with lancets and an engaged feed stop mechanism.

FIG. 42 shows a magazine group after filling with lancets, connection of a head group, and disengagement of a feed stop mechanism.

FIG. 43 shows a lateral cross-section of a dispenser bulk loader filled with test strips inserted into a magazine group.

FIG. 44 shows a lateral cross-section of a dispenser bulk loader inserted into a magazine group loading test strips.

FIG. 45 shows a lateral cross-section of a dispenser bulk loader inserted into a magazine group when loading test strips is completed.

FIG. 46 shows a horizontal cross-section of a magazine group having feed regulators and a feed stop mechanism.

FIG. 47 shows a magazine group filled with test strips and an engaged feed stop mechanism.

FIG. 48 shows a magazine group after filling with test strips, connection of a head group, and disengagement of a feed stop mechanism.

FIG. 49 shows a dispenser for dispensing lancets and test strips in working pairs.

FIG. 50 shows a lateral cross-section of the dispenser of FIG. 49.

FIG. 51 shows a dispenser having a transparent view window and graduations indicating the number of tests supplies remaining in the dispenser.

FIG. 52 shows a carrier strip to which individual test strips have been affixed.

FIG. 53 shows a carrier strip to which individual lancets have been affixed.

FIG. 54 shows a carrier strip to which individual lancets have been affixed forming a bandolier.

FIG. 55 shows a carrier strip to which lancets and test strips have been affixed in working pairs.

FIG. 56 shows a carrier strip to which testing supplies have been affixed wound around a dispensing spool.

FIG. 57 shows a partial cut-away top frontal perspective of a dispenser for testing supplies affixed to a carrier strip.

FIG. 58 shows a top frontal perspective of a dispenser for testing supplies affixed to a carrier strip.

FIG. 59 shows a longitudinal cross-sectional detail of a carrier strip cutting blade and actuator.

FIG. 60 shows a storage receptacle for used lancets and test strips and an intact ampule containing a sterilizing fluid.

FIG. 61 shows a storage receptacle for used lancets and test strips and a fractured ampule containing a sterilizing fluid.

FIG. 62 shows a top frontal perspective of a dispenser having a thumb wheel actuator and port adapted to test strips.

FIG. 63 shows a top frontal perspective of a dispenser having a thumb wheel actuator and port adapted to lancets.

FIG. 64 shows a cross-section of a dispenser having a thumb wheel actuator and drum ejector adapted to lancets.

FIG. 65 shows a cross-section of a dispenser having a thumb wheel actuator and drum ejector adapted to test strips.

FIG. 66 shows a top frontal perspective of a case for a testing supply dispenser.

FIG. 67 shows a cross-section of a dispenser for testing supplies also having receptacles for used testing supplies.

FIG. 68 shows a cross-section of a dispenser having a dispenser cylinder.

FIG. 69 shows a cross-section of a dispenser having a dispenser cylinder head group, a slide actuated ejector, and a magazine group attachment.

FIG. 70 shows a dispenser with an integrated lancing device.

FIG. 71A shows a detail of the lancing device of FIG. 70.

FIG. 71B shows a detail of the port regulator interacting with a lancet during lancing.

FIG. 71C shows a detail of the port regulator blocking a used lancet from being reinserted into a dispenser.

FIG. 72 shows the dispenser of FIG. 70 prepared for lancing.

FIG. 73 shows the dispenser of FIG. 70 after firing the lancing device.

FIG. 74 shows the dispenser of FIG. 70 after a lancing operation has been completed.

FIG. 75 shows the dispenser of FIG. 70 dispensing a used lancet and cocking the lancing device.

### DETAILED DESCRIPTION OF THE INVENTION

Dispensers may be constructed to hold large quantities of blood glucose test strips (hereafter referred to as “test strips”) in an orderly and sanitary manner. Generally, such dispensers are constructed using a magazine or similar holding volume that contains a number of test strips in a sealed environment. These dispensers are also provided with a dispensing mechanism that conveniently delivers individual strips from the dispenser magazine when they are needed. These same dispenser embodiments also lend themselves to use with needle lancets and provide an orderly, sterile, and secure packaging and delivery method.

Dispensers may be pre-filled by test strip manufacturers and offered for sale as a complete unit. In other embodiments, the dispensers are reloadable. In reloadable embodiments, consumers may purchase a dispenser and quantities of test strips or lancets sealed in foil packs or similar low cost packaging separately. The test strips and lancets in these embodiments are further prepared for bulk loading into the dispenser so the patient does not have to deal with individual testing supply elements. Thus, reloadable dispenser embodiments reduce production costs for the manufacturer over time, and provide the consumer with an improved system for storing and managing their testing supplies.

Other dispenser embodiments are specifically sealed for single use, and include tamper-resistant elements that render the strips useless when attempts to open the dispenser are made. These embodiments are particularly directed at test strip supplies that are provided through subsidized funding programs, and act as a deterrent to resale of excess, or fraudulently acquired, test strips and lancets. These embodiments may also include tamper-resistant elements in the form of marking devices that discourage simple emptying of the dispenser in the intended manner, and repackaging the dispensed test strips or lancets for resale.

Embodiments of dispensers for lancets provide many of the same benefits as the dispensers for test strips. Lancets may be readily dispensed individually as needed, and loss, fraud, and adulteration can be minimized.

In some embodiments, test strip dispenser structures, lancet dispensing structures, and (optionally), receptacles for storage of used strips and lancets, may be integrated into a single dispenser that provides all of the benefits of individual dispensers. These embodiments may also be constructed to force the patient to dispense one test strip with each lancet, encouraging them to follow sterile procedures with regard to single-use of the lancets.

In some embodiments, lancet and test strip dispensers may be adapted to couple directly to lancing devices and blood glucose meters, respectively, so that these single use components are dispensed directly into their multi-use host devices when prepping for a test. These embodiments may be advantageous to young patients, elderly patients, and others who may have difficulty handling the small disposable components and properly inserting them into their host devices (blood glucose meters, and lancing devices).

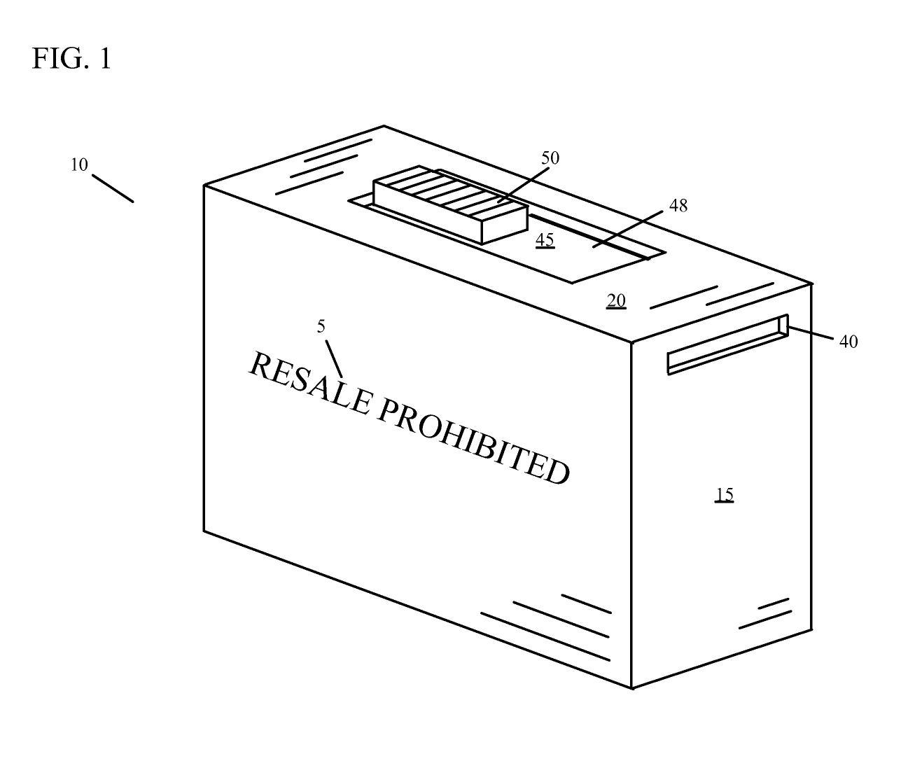

One embodiment of a test strip dispenser 10 is shown in FIG. 1. The dispenser 10 has a generally rectilinear case in this embodiment. The case top 20 has a slot 48 formed in it that allows a slide 45 to move longitudinally within the dispenser. The slide 45 is also preferably provided with an actuator grip 50 that the patient may apply force to in order to actuate the slide 45 when dispensing a test strip. The front 15 of the dispenser 10 is provided with a port 40 through which a test strip passes from the inside of the dispenser 10 for delivery to the patient when the slide 45 is actuated.

Throughout the description and figures of the various dispenser embodiments described herein, the port 40 is generally shown on the front 15 of the dispenser 10, with the slide 45 and actuator grip 50 (or other actuator mechanism) oriented to move a testing supply from back to front within the dispenser 10. This representation should not be considered limiting of the embodiments described herein, as the various elements (e.g., slide 45, grip actuator 50) that move individual testing supplies may also be arranged to move supplies laterally in any direction (front to back, back to front, or from one side to the other) to a port 40 appropriately placed to allow the testing supply to exit the dispenser when the dispenser 10 is operated. Further, the same or similar elements are referred to with the same reference numerals throughout all the figures.

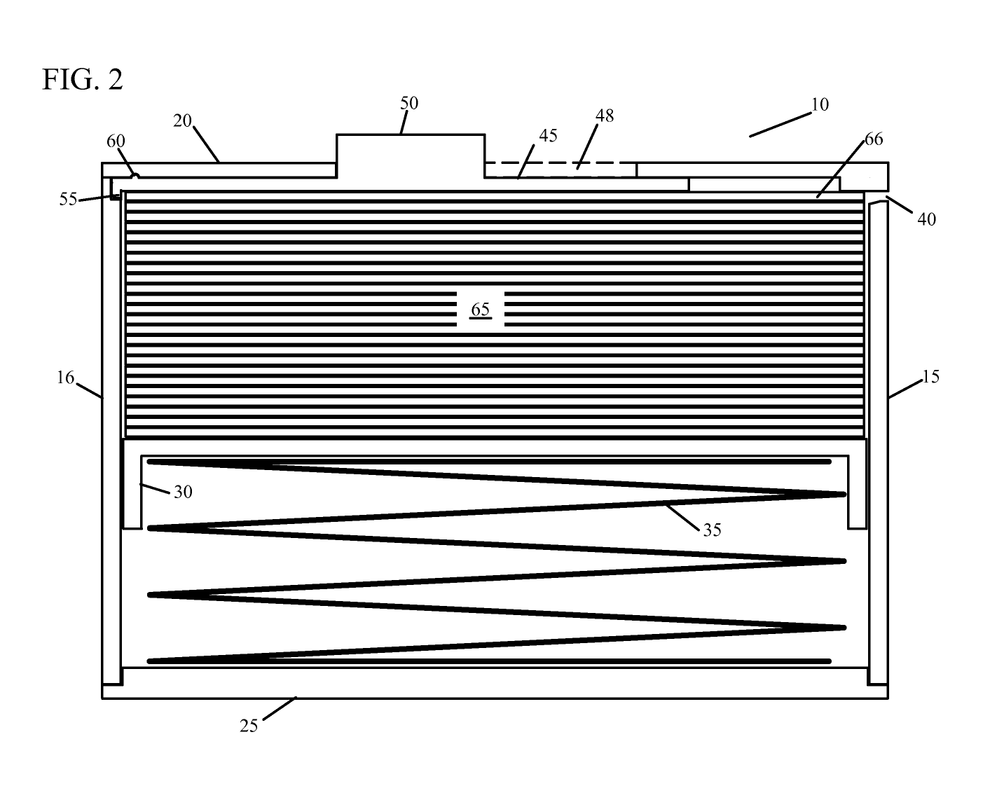

FIG. 2 shows a cross-section of the dispenser 10 shown in FIG. 1, from the front 15 of the dispenser to the back 16 of the dispenser. The volume enclosed by the top 20, bottom, 25, front 15, back 16, and sides of the dispenser forms a magazine and a storage volume which holds a stack 65 of test strips. The stack 65 is forced upward against the slide 45 and dispenser top 20 by a magazine spring 35 pushing on a follower 30 that supports the bottom of the test strip stack 65. The magazine spring 35 may be of any construction that biases the follower toward the slide 45 and dispenser top 20, including but not limited to, coil springs, saw-tooth shaped flat metal springs, or other similar structures.

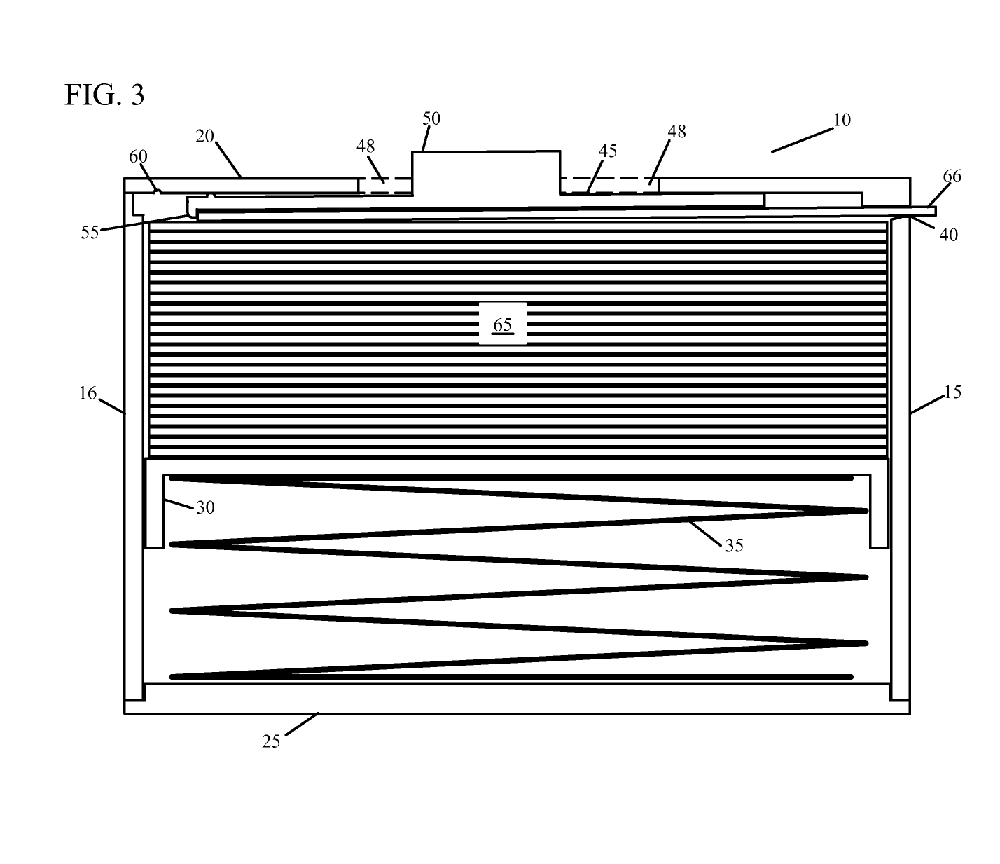

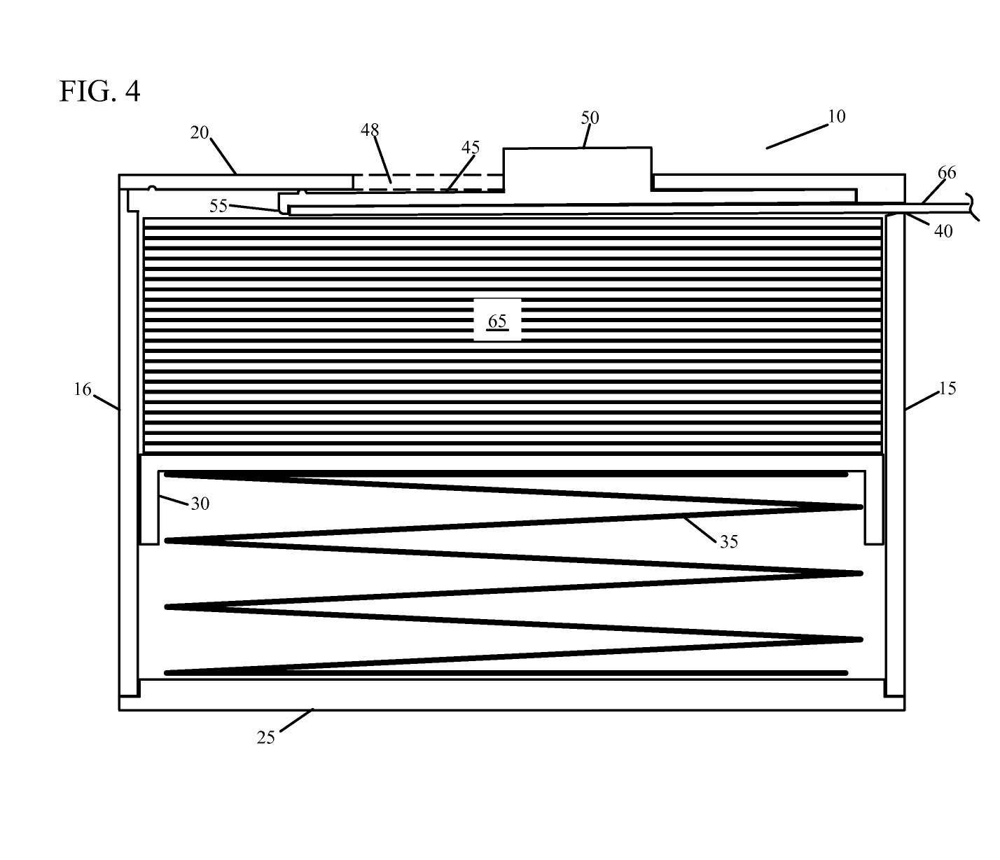

In a ready state, the slide 45 is held in place at the rear of the dispenser 10 by a detent 60 that resists forward motion of the slide. In this condition, an ejector 55 extending downward into the dispenser 10 is held to the rear of the test strip stack 65. The ejector 55 is dimensioned vertically to approximately the thickness of a single test strip and horizontally to approximately the width of a test strip. When the dispenser 10 is operated by pressing the actuator grip 50 and slide 45 toward the front wall 15 of the dispenser 10, as shown in FIGS. 3-4, the ejector 55 pushes a single test strip 66 forward, toward the front wall 15 of the dispenser 10, and out of the dispenser 10 through the port 40 in the front wall 15.

In operation, the follower 30 is designed to fit inside the storage volume of the dispenser 10 so that it will slide freely throughout the length of the magazine, but not tilt substantially. The follower 30 may then apply even pressure to the test strip stack 65 as it is pressed downward by the motion of the detent 60, and in turn depress the magazine spring 30 without binding. Similarly, the ejector 55 maintains downward pressure on the stack 65 as the slide 45 moves forward across the rest of the stack 65 as a single test strip 66 is ejected through the port 40.

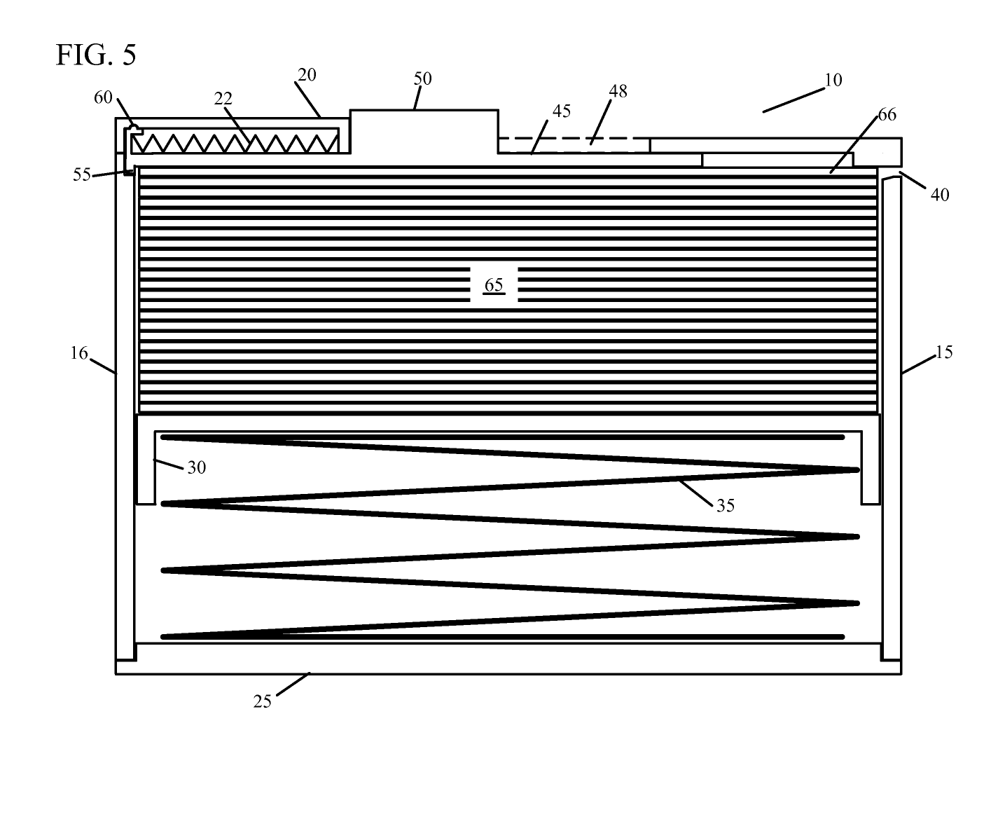

When the slide 45 is moved to its most forward position in the dispenser 10, as shown in FIG. 4, the single test strip 66 dispensed is held in place by the force of the magazine spring 35 through the follower 30 and stack 65, pinching the test strip 66 against the slide 45 and dispenser top 20. In this position, the patient may conveniently grasp and remove the test strip 66. After the patient has removed the dispensed test strip 66, the dispenser 10 is brought back to the ready position of FIG. 2 by returning the actuator grip 50 and slide 45 to their rearmost position. This return operation may be performed manually by the patient, or as shown in FIG. 5, by a return spring 22 incorporated to return the slide 45 to the ready position when pressure is released from the actuator grip 50. The stack 65 is then free to move upward through the action of the magazine spring 35, and bring another test strip 66 into a top-most position to be dispensed upon the next actuation of the dispenser 10.

In some embodiments, the dispenser 10 shown in FIGS. 1-5 is produced in three pieces including a top 20, a bottom 25, and a tube forming the front 15, back 16, and sides of the dispenser. Alternatively, the dispenser 10 is formed as a cup in which, for example, only the top 20 or bottom 25 need be attached at the end of assembly. In one embodiment, where the top 20 is already in place and the bottom 25 is open, the dispenser 10 is easily assembled by inserting the slide 45, charging the dispenser with a stack 65 of test strips 66, inserting the follower 30 and follower spring 35, and attaching the bottom 25. The reverse order is also possible in constructions where the bottom 25 has been added first, and assembly is completed by adding the top 20.

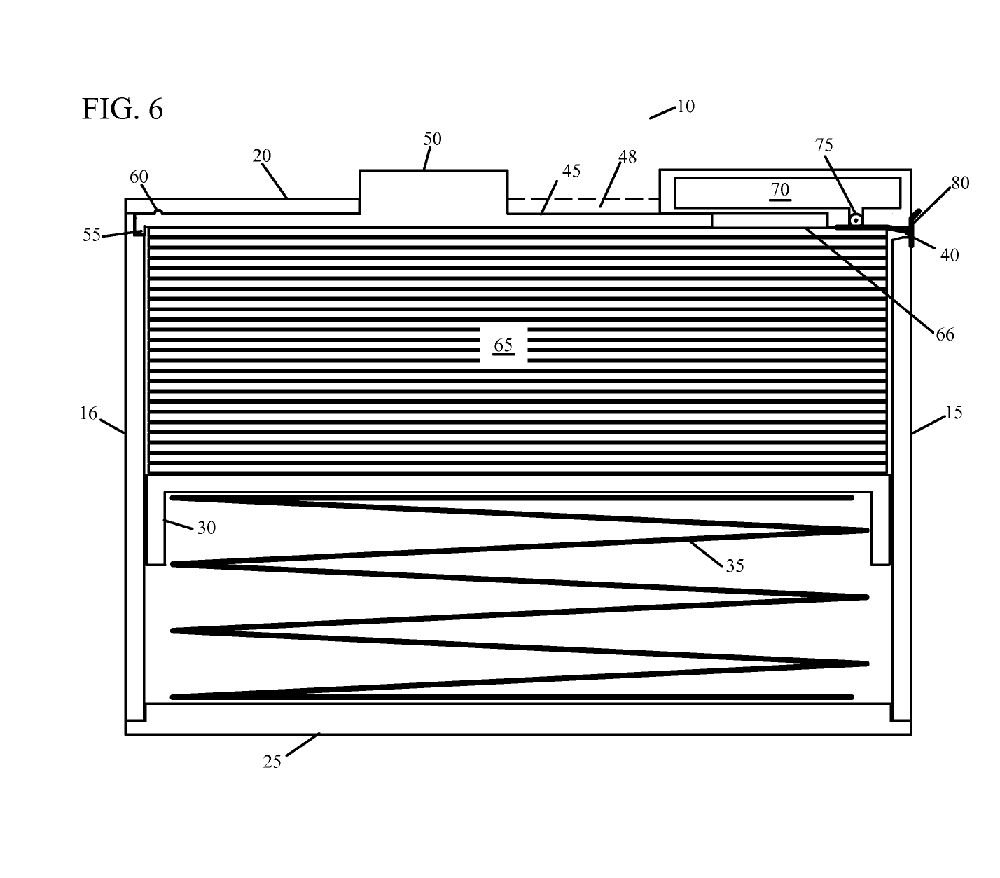

In one preferred embodiment, shown in FIG. 6, the dispenser 10 may be fitted with tamper-resistant elements that discourage resale of unused test strips. In this embodiment, the dispenser 10 tamper-resistant element is an applicator 75 that comes in contact with the test strip 66 at the top of the stack 65. The applicator 75 is further in communication with a reservoir 70 in the dispenser 10 that contains a permanent dye, ink, or other marking fluid that may be used to indelibly mark test strips 66 as they are dispensed. The marking applied may be of any type, including but not limited to, a stripe, a geometric figure, and/or alphanumeric characters.

In some embodiments, the marking fluid is biocompatible. The marking fluid is preferably formulated to be rapidly absorbed into the surface of the test strip, and to change over time. For example, at first application, the marking fluid may appear blue, but over a period of time, for example a few hours, change to red. Thus, used test strips, or strips that have been simply dispensed for the purpose of resale to others can be discouraged without confusing the legitimate user when the test strips are dispensed. The marking fluid may be reactive to elements on the surface being marked, part of a two part dye formulation, may be reactive to light over time (photo-sensitive), or may be reactive to oxygen or other environmental elements that cause it to change color.

The applicator 75 may be a roller, an absorbent felt, or other suitable structure for applying the contents of the reservoir 70 to the dispensed test strip 66. Placement of the applicator 70 is preferably chosen to prevent it from contacting the active open blood collecting end of the test strip 66 during dispensing to avoid introducing the marking fluid into the blood collection tube.

The reservoir 70 and applicator 75 may be sealed during manufacture using a safety seal 80 that also covers the port 40. In this embodiment, test strips may not be dispensed until the safety seal 80 is removed, which simultaneously allows the applicator 75 to come in contact with the test strip 66 at the top of the stack 65.

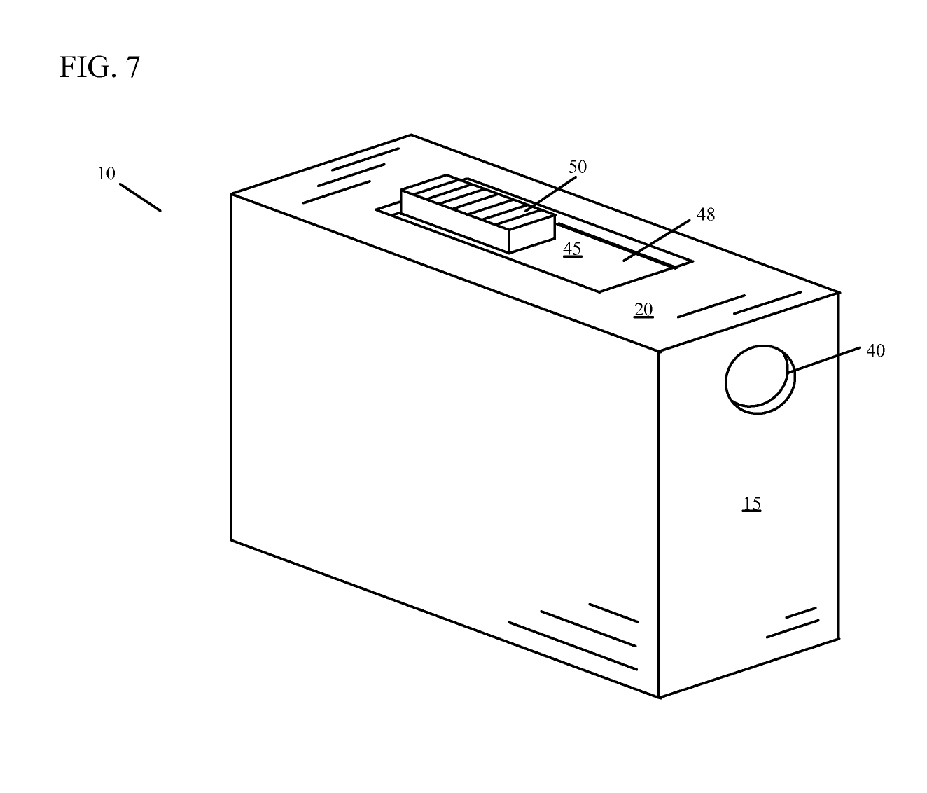

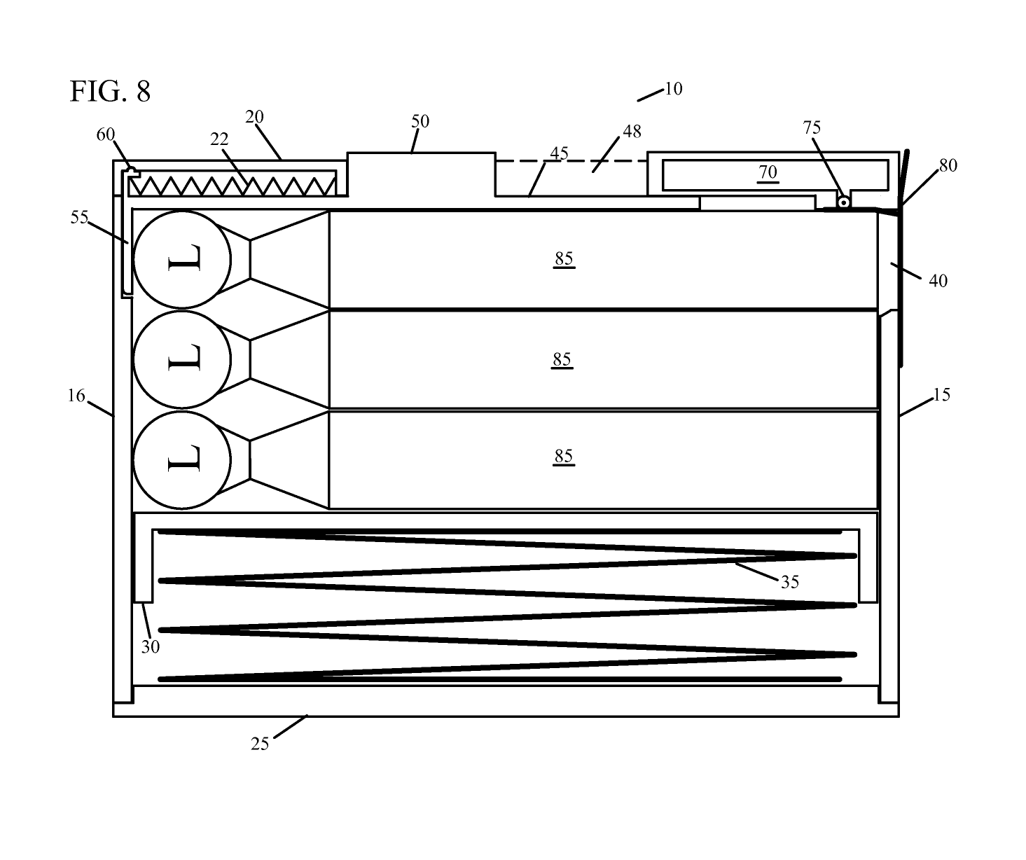

The dispensers 10 shown in FIGS. 1-6 are also readily adaptable to dispensing lancets, as shown FIGS. 7-11. FIG. 7 shows a front perspective of a dispenser 10 adapted for use with disposable needle lancets 85 that are substantially cylindrical in form. To accommodate this embodiment, the port 40 is a circular hole in the front 15 of the dispenser 10. Referring to FIG. 8, when dispensing disposable needle lancets 85, the bottom 25, follower 30, magazine spring 35, and elements associated with dispensing such as the slide 45, and actuator grip 50 remain substantially unchanged in comparison to the dispensers 10 shown in FIGS. 1-6. However, to accommodate the larger size of the disposable needle lancets 85, the ejector 55 is preferably lengthened, and the width of the magazine and ejector 55 are increased accordingly. As with other embodiments, a return spring 22 may be added, as well as tamper-resistant elements such as a marking fluid reservoir 70 and an applicator 75.

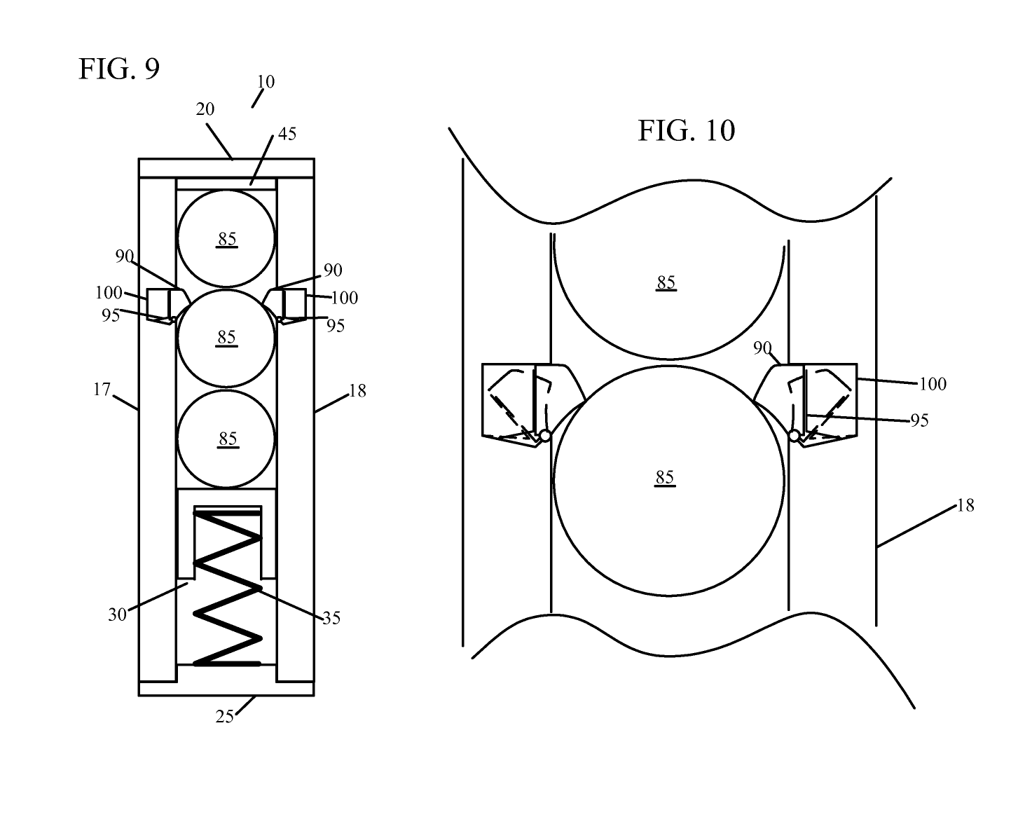

While disposable needle lancets 85 are generally cylindrical throughout most of their length, in some embodiments the lancets 85 may have irregularities, such as removable needle shields, that may inhibit smooth operation of the slide 50 from its ready position, to full dispensing position, and back to ready position. As shown in FIGS. 9-11, feed regulators in the form of spring-loaded sears 90 may be incorporated within recesses 100 in the side walls 17, 18 of the dispenser 10 to ensure smooth operation of the slide 45 throughout its operating range.

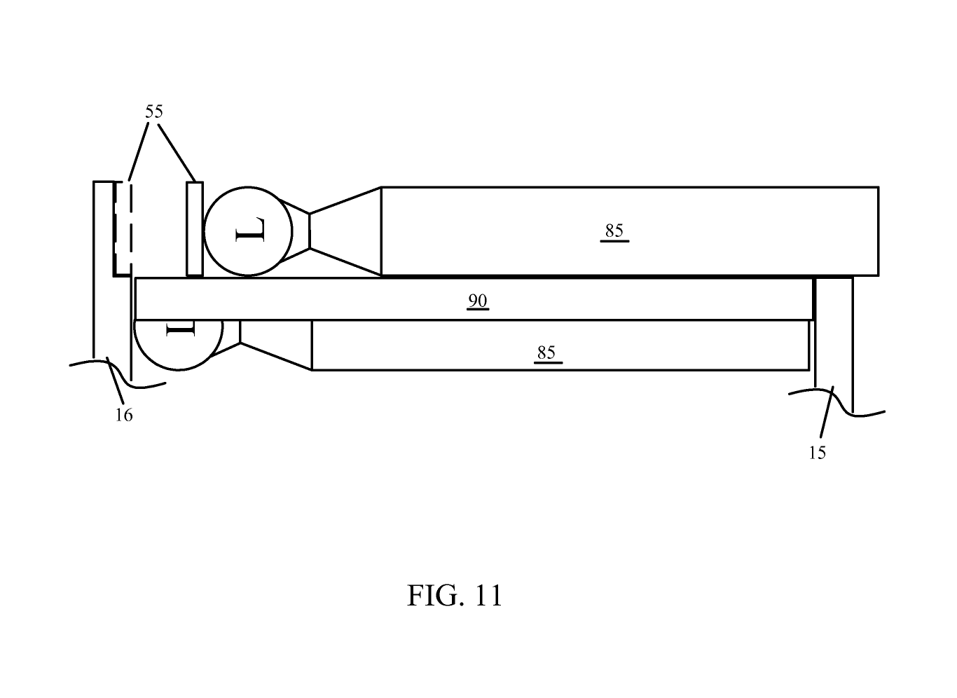

When a disposable needle lancet 85 is in the ready position under the slide 45, it holds the other disposable needle lancets 85 in the magazine down against the follower 30 and force of the magazine spring 35. In this condition, the spring-loaded sears 90 are also held in their extended position within the storage volume by springs 95. If the ready position lancet 85 (top-most in stack) is removed, and the ejector 55 is in any position except its most rearward, ready position (dashed lines in FIG. 11), the spring-loaded sears 90 are held in their extended position by the lower edge of the ejector 55 (see FIG. 11) riding along their upper surface.

Thus, even when the upper lancet 85 is fully removed by the patient, upward motion of the remaining lancets 85 is blocked, and the ejector 55 may still be smoothly operated regardless of irregular surface features on the lancets 85. When the ejector 55 is returned to its rearward most position (dashed lines in FIG. 11), the spring-loaded sears 90 are no longer prohibited from pivoting out of the feed path (dashed lines in FIG. 10) in the storage volume, and the remaining disposable needle lancets 85 may move upward under the force of the magazine spring 35, delivering another lancet 85 into the ready position. Proper angulation of the upper surface of the spring-loaded sears 90 also allows them to be pushed out of their blocking position when a stack of disposable needle lancets 85 is pushed into the dispenser 10 from above.

The dispenser 10, and its tamper-resistant elements 70, 75, 80, have been described with regard to linear feed magazines for both test strips 66 and lancets 85. However, alternative feed geometries are possible for both test strips 66 and lancets 85 utilizing the same slide 45 and ejector 55 mechanisms.

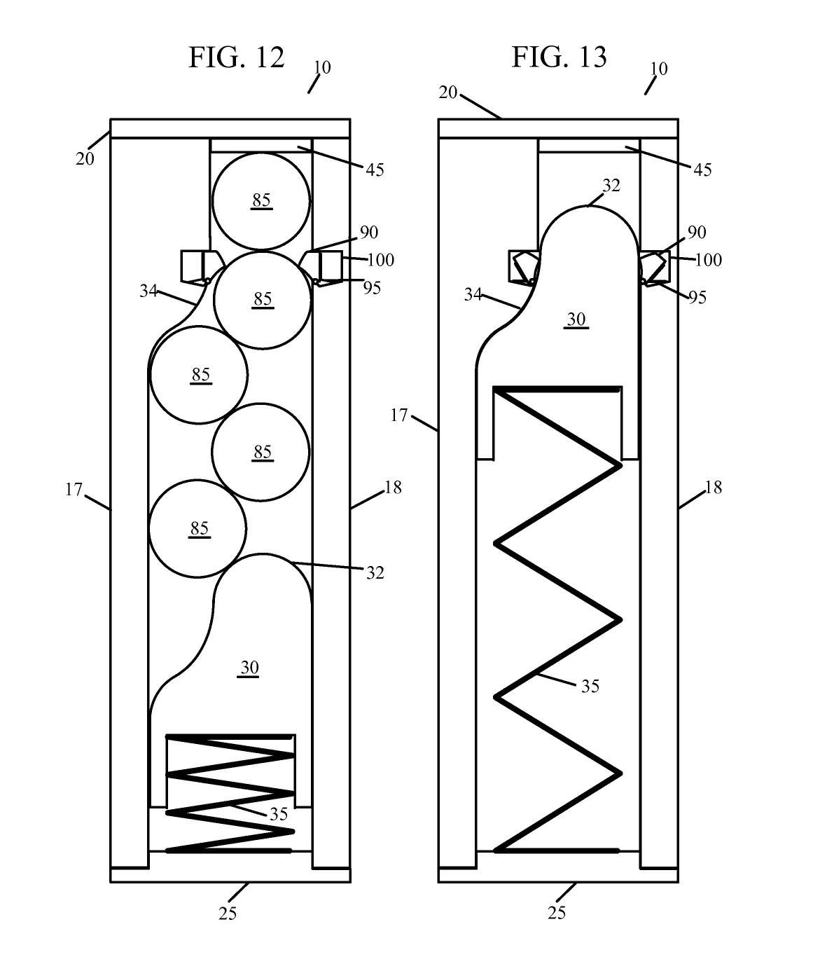

FIGS. 12-13 show a staggered stack magazine. In this embodiment, the width of the magazine volume is increased to allow the lancets 85 to alternate between the left side 17 and right side 18 of the dispenser 10 storage volume. This arrangement allows more lancets 85 to be stored in the same length dispenser 10. As with the embodiment shown in FIGS. 9-10, spring-loaded sears 90 are preferably incorporated in the upper aspect of the dispenser 10 to allow for smooth slide 45 and ejector 55 (not show in this cross-section) operation regardless of irregularities in the shape of the specific lancets 85 being dispensed.

In this embodiment, the follower 30 has been formed with an extension 32 that simulates the presences of a lancet 85 at the lowest point in the magazine, maintains the lateral position of the lowest lancet 85 in the magazine, and forces it into position under the slide 45 when all other lancets have been dispensed. A guide ramp 34 is also preferably incorporated on the interior of the side 17 of the dispenser 10 to transition the lancets 85 in the staggered stack toward the spring-loaded sears 90, slide 45, and ejector 55 as they are sequentially dispensed. While the guide ramp 34 is shown on the left side 17 of the dispenser 10, it may alternatively be placed on the right side 18, or a guide ramp 34 may be placed on each side 17, 18 of the dispenser, directing the top most lancet 85 to the center of the dispenser 10. In each case, the follower 30 extension 32 is appropriately modified to force the last lancet 85 along the guide ramp(s) 32, and into a dispensing position.

As can be seen in FIG. 13, when all lancets 85 have been dispensed, the follower 30 extension 32 moves past the feed regulators and into the space from which a lancet 85 would normally be located in the ready position when the slide 45 and ejector 55 are returned to their rearmost position in the dispenser 10. This localization of the follower 30 extension 32 then blocks further actuation of the slide 45, giving a clear indication that the dispenser is empty.

The linear feed magazine embodiments shown in FIGS. 7-13 depict a small number of lancets 85 contained in the dispenser 10. This depiction should not be considered a limitation and is only used for clarity. The length of the dispenser 10 may be varied to accommodate large numbers of lancets in both linear feed embodiments, with appropriate modifications of the magazine spring 35.

FIG. 14 shows a serpentine dispenser 10. In this embodiment, a center wall 19 is constructed between the left wall 17 and right wall 18 of the dispenser 10. The lower end of the center wall 19 has been shaped as a semi-circle to allow smooth feeding of the lancets 85, follower 30, and follower spring 35 throughout the entire feed channel. Similarly, the internal surface of the dispenser 10 bottom has been formed as a semi-circle to maintain a continuous serpentine feed channel through which the lancets 85, follower 30, and follower spring 35 may transit without jamming or blockage. In this embodiment, the follower 30 is formed as a cylinder to allow it to negotiate curves in the serpentine dispenser magazine feed path.

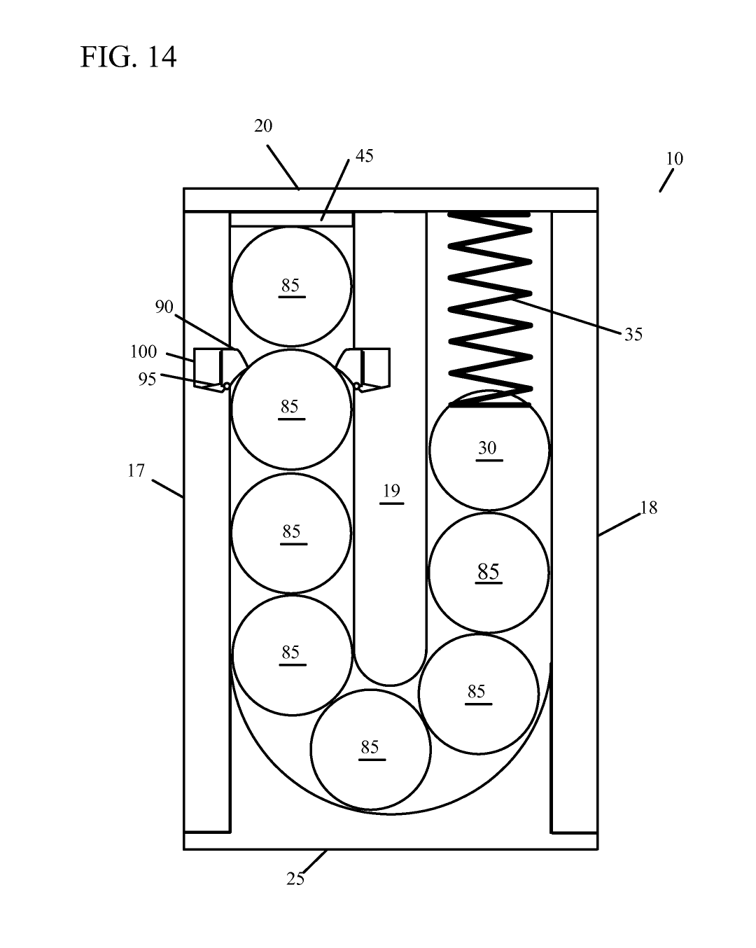

In other embodiments, the magazine feed pathway inside the dispenser 10 may be radially oriented as a circle, shown in FIG. 15 and FIG. 16, a planar spiral, or a helix. FIG. 15 shows an embodiment for dispensing lancets 85, and FIG. 16 shows an embodiment for dispensing test strips 66. As with the embodiments of FIGS. 1-14, dispensing is achieved by operating an actuator grip 50 attached to a slide 45 that has an ejector 55 which pushes a test strip 66 or a lancet 85 out of a port on one side of the dispenser 10. The tamper-resistant marking fluid reservoir 75, applicator 75, and return spring 22 of FIGS. 1-14 may also be incorporated into the embodiments shown in FIGS. 15-16. All of these elements are incorporated to intersect with the radial magazine feed path and function in the same manner as described previously for FIGS. 1-14.

FIG. 15 shows a circular feed path formed by an outer wall 14 and an inner wall 13. Equally these walls 13, 14 may form the inner and outer walls of a planar spiral or helix. A cylindrical follower 30 is used to allow smooth feeding of the lancets 85 through the arcuate feed pathway of the magazine without binding. As the magazine spring 35 is flexible, it can readily bend along this same pathway as well. FIG. 16 shows a similar embodiment, but dimensioned for, and filled with, a stack 65 of test strips 66. As the individual test strips 66 are relatively thin and narrow, the stack 65 easily curves to follow the arcuate pathway within the dispenser 10. In this example, a generally cylindrical follower 30 has been modified with a flat facing the stack 65 of test strips 66 to ensure that the bottom strips do not tilt and jam in the dispenser 10 magazine.

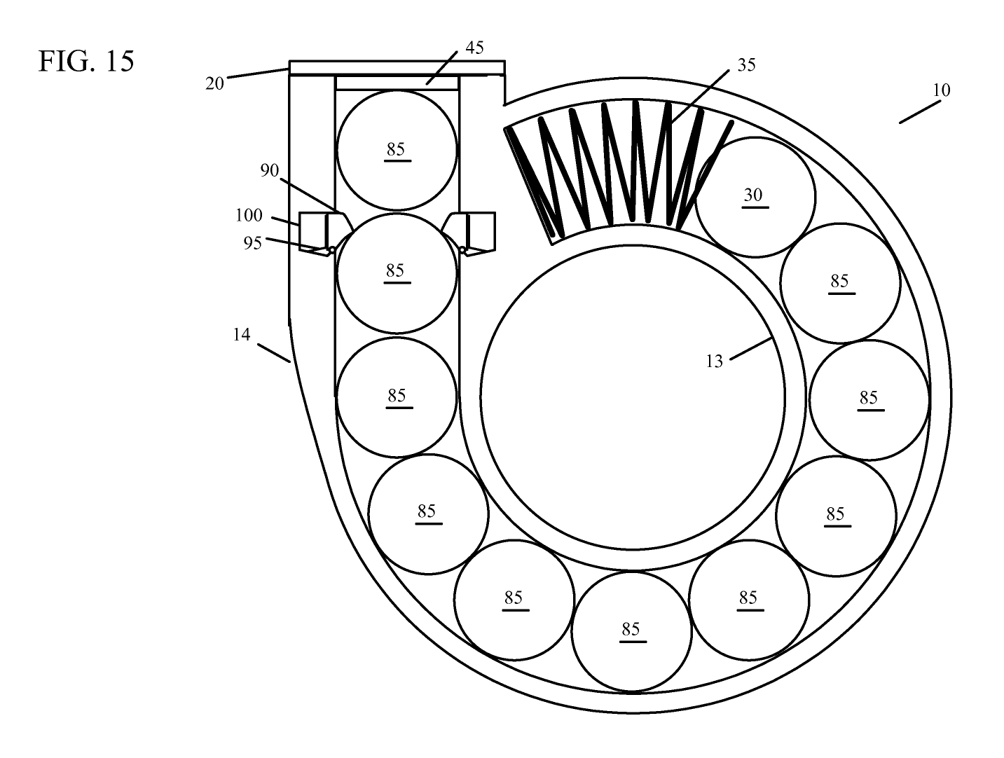

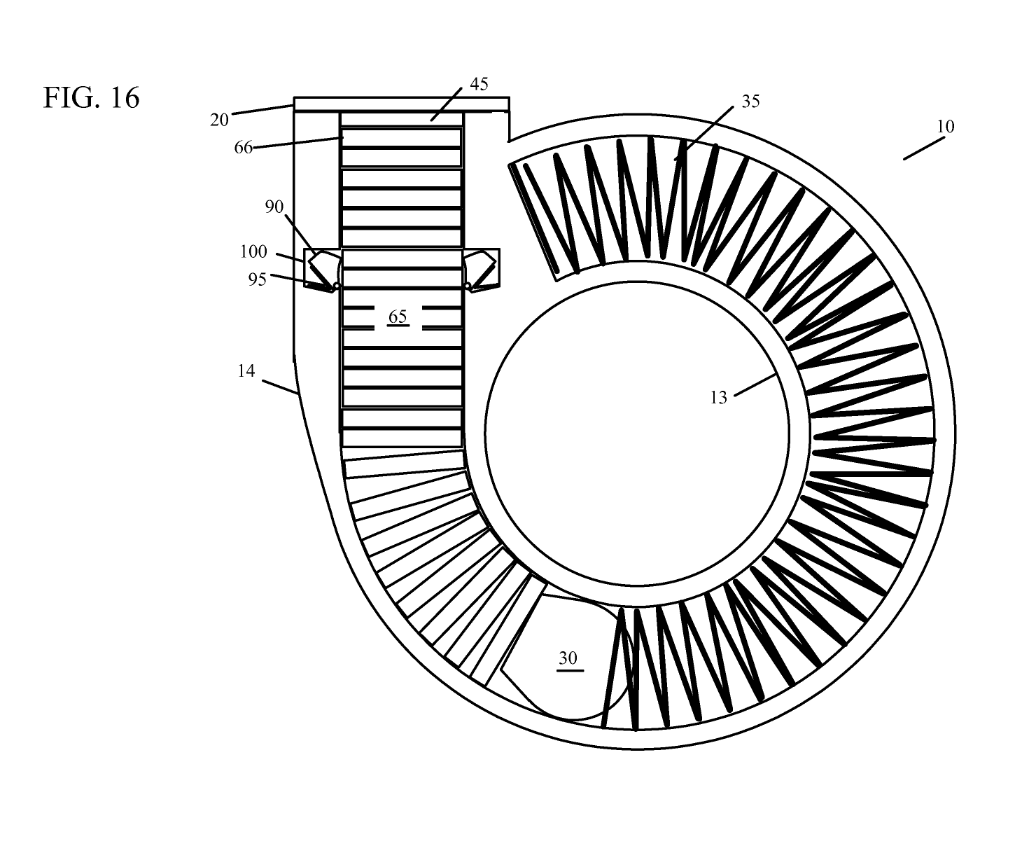

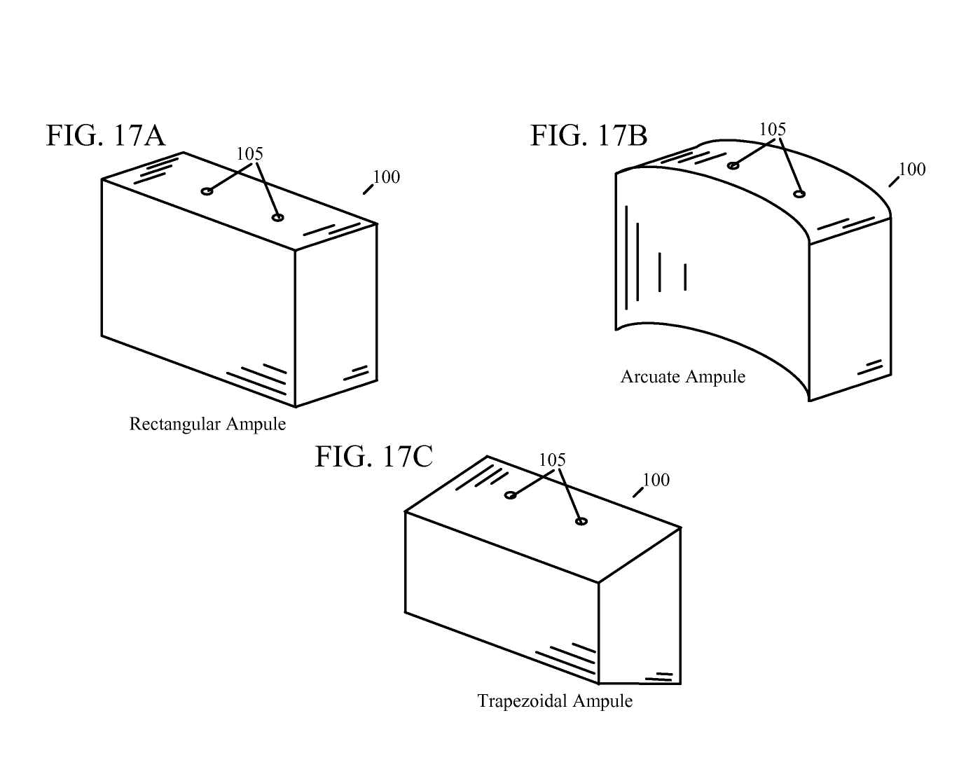

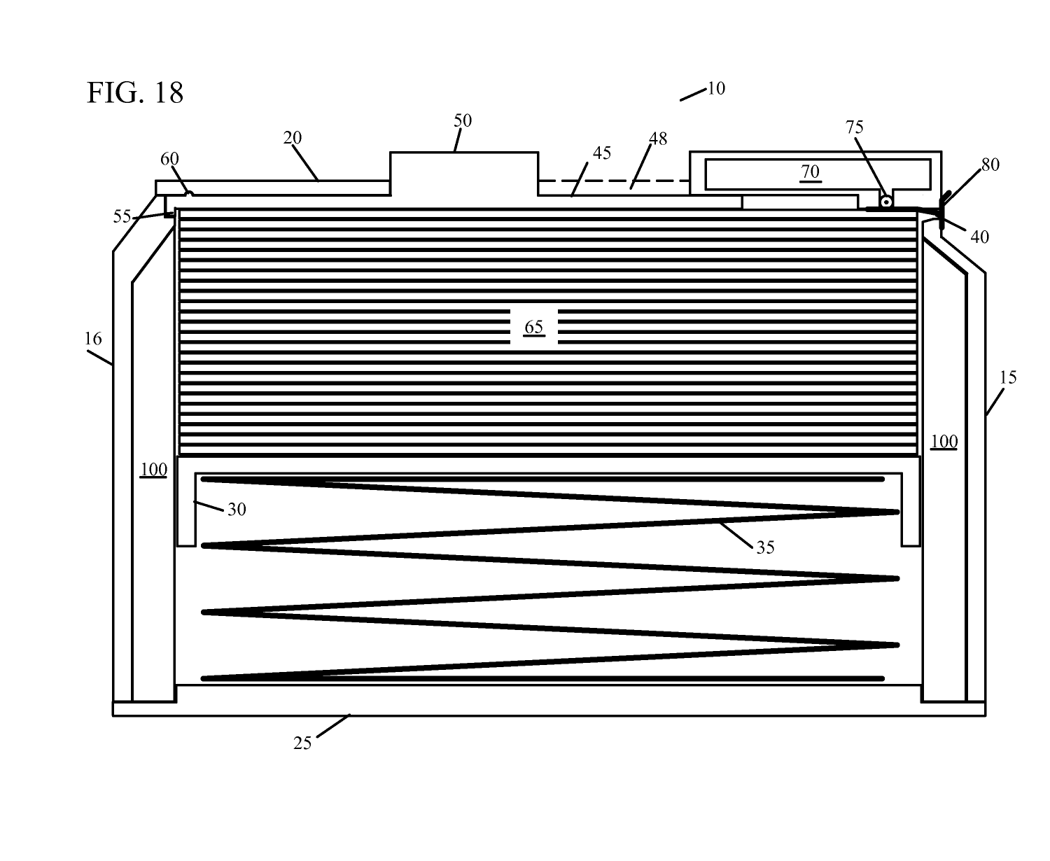

The embodiments of FIGS. 1-16 are sealed dispensers 10, all of which may be provided with tamper-resistant elements such as applicators 75 and marking fluid reservoirs 75, as particularly shown in FIG. 6. Additional tamper-resistant elements, shown in FIGS. 17-18, may also, or alternatively, be added to any or all of these embodiments in the form of a breakable ampule 100. Common to all embodiments of the ampule 100 are the properties of the material it is constructed from, and the fluids it contains.

The ampule 100 is constructed of very thin materials that have a higher brittleness (defined by the degree of plastic deformation prior to fracture) than the material from which the dispenser 10 case is constructed. For example, the ampule 100 may be formed as a thin walled vessel from polymethyl methacrylate (PMMA), polystyrene, and other materials that exhibit little or no plastic deformation before fracturing. During construction of the dispenser 10, one or more ampules 100 may be strategically attached to the inner surfaces of the dispenser 10, or within open recesses as may be most appropriate in a given situation. Attachment may be affected through the use of cyanoacrylate adhesives, ultrasonic welding, or similar adhesion techniques. Alternatively, guide structures may be formed in the inner surfaces of the dispenser 10 walls 13, 14, 15, 16, 20, 25 and ampules 100 may be inserted into these guide structures during assembly.

FIGS. 17A-17C show three embodiments of an ampule 100 that may easily be integrated into the internal space of a dispenser 10 at various locations. In FIG. 17A, the ampule 100 is shown as a simple hollow rectilinear form. FIG. 17B shows an arcuate form, and FIG. 17C shows a trapezoidal form. However, the ampule 100 may be produced with various hollow shapes, and have sealed channels passing through it to accommodate different dispenser 10 geometries, and access portals through the dispenser 10 case. The ampule 100 may be constructed with fill holes 105 through which fill nozzles (e.g., needles) introduce a fluid at the time of manufacture. These fill holes 105 may be permanently sealed using fast setting glue (e.g., cyanoacrylate), ultrasonic welding, permanent tape, or other similar methods after filling.

FIG. 18 shows an embodiment of the dispenser 10 of FIG. 6 in which ampules 100 have been integrated in recesses in the front and rear sides 15, 16 of the dispenser 10. In addition to being adhered to the inside surfaces of the front and rear 15, 16 of the dispenser 10, the bottom of the ampule 100 is positioned so that when the bottom 25 of the dispenser 10 is attached to the dispenser 10 through adhesives, ultrasonic welding, or other permanent means, the dispenser 10 bottom 25 is also permanently bonded to the bottom of the ampules 100. As a result, any attempt to remove the bottom 25 will also cause the ampules 100 to fracture, and expose the test strips 66 to the fluid contained in the ampule 100.

As the ampules 100 are rigidly held by the dispenser 10 case, any attempt to twist, deform, fracture, separate, or otherwise gain entry into the dispenser 10 will cause the ampule 100 to fracture before the material of the dispenser 10 housing does (as the ampule 100 does not exhibit plastic deformation and the materials chosen for the dispenser 10 housing do to a greater degree). Upon fracturing, shattering or otherwise breaking, the ampule 100 will release its contents into the interior of the dispenser 10.

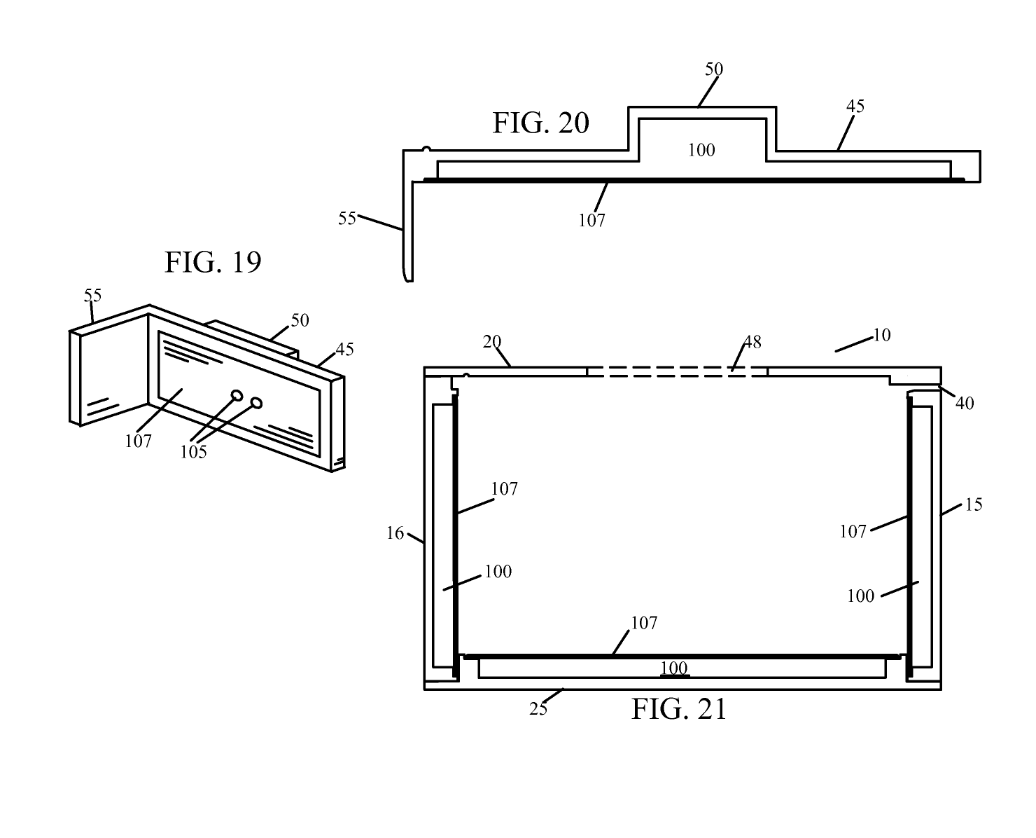

In other embodiments, shown in FIGS. 19-21, the ampule 100 structure is integral to one or more elements of the dispenser 10. For example, as shown in the lower perspective of a slide 45 (FIG. 19), and the cross section of that slide 45 and actuator handle 50 (FIG. 20), the dispenser 10 slide 45 may be constructed with a hollow that is then covered by an ampule 100 plate 107. This plate 107 is similarly constructed of a thin sheet of material that is more brittle than the material from which the slide 45 is constructed. The plate 107 may be affixed in a recess to form a uniform surface on the slide 45 using cyanoacrylate adhesives, ultrasonic welding, or other similar bonding methods that result in a fluid-tight joint. Filling holes 105 in the plate 107 then allow the ampule 100 to be injected with a fluid, and may be sealed using cyanoacrylate adhesives, ultrasonic welding, permanent tapes, or other permanent fluid tight methods.

FIG. 21 shows a dispenser 10 case in which the bottom 25, front 15, and back 16 of the dispenser 10 are constructed with integral ampules. Sides 17, 18 may be similarly prepared, but are not visible in this cross-section.

As previously described, the inside of the dispenser 10 bottom, front 15, and back 16 are constructed with hollows and recesses in which plates 107 may be adhered to form a sealed ampule 100. In the case of the front 15 and back 16 walls (and walls 17, 18 that are not shown), the ampule 100 plates 107 extend downward to overlap with the joint between the bottom 25 and the walls 15, 16. As a result, attachment of the bottom 25 to the walls 15, 16, 17, 18 also causes the bottom 25 to adhere to the plates 107 on those walls 15, 16, 17, 18. Thus, any attempt to remove the bottom 25 will cause a fracture of the ampule 100 plates 107 in the bottom 25, front 15, back 16, and/or sides 15, 16.

In any of the previously described ampule 100 embodiments, the space within the dispenser 10 will be flooded with the fluid the ampules 100 contain, indicating they have been tampered with in some manner, when attempts to open the dispenser 10 are made. The top 20 may also, or alternatively, be constructed with integrated ampules 100, but has not been shown here for simplicity. These examples of integrated ampules 100 in a linear feed magazine dispenser 10 and slide 45 are not intended to be limiting of this construction, as the technique may equally be applied to any of the components and configurations discussed herein.

The fluid contained in the ampule 100 may include an indelible dye solution, a reagent that reacts with a coating on the testing supply 66, 85 to change color, a protein solution, an ascorbic acid solution, a combination thereof, or other solutions that would permanently mark test strips 66 and lancets 85, and render test strips 66 unusable. An ascorbic acid solution clogs and/or alters the capillary tubes that draw blood into a test strip 66 so that the capillary tubes no longer function. A protein solution reacts with active ingredients contained in test strips 66 so that they no longer perform in their intended manner. In preferred embodiments, the ampule contains two parts protein solution, and one part ascorbic acid solution. However, other protein/ascorbic acid combinations are possible, and other solutions that disable the test strip 66 capillary tubes and/or active ingredients may also be substituted.

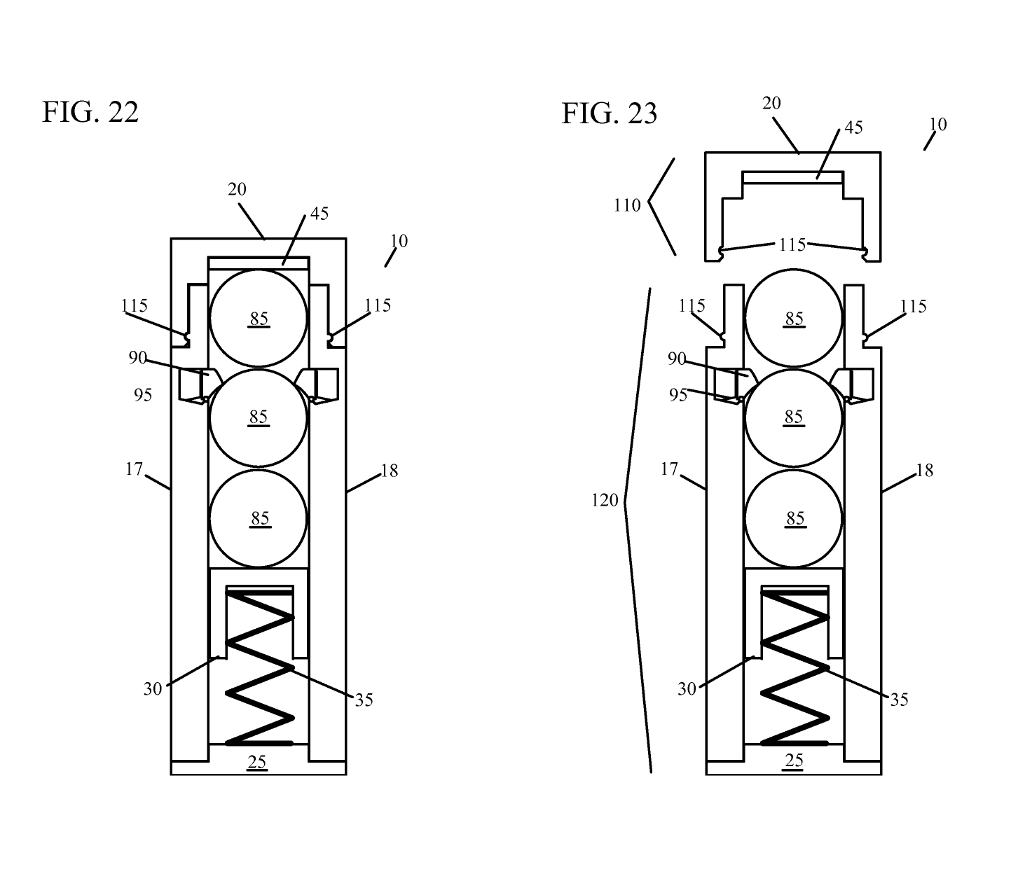

The dispenser 10 embodiments described in FIGS. 1-21 are sealed, single use, dispensers 10 that are pre-stocked with testing supplies 66, 85 at the point of manufacture. In other embodiments, shown in FIGS. 22-26 and FIGS. 62-65, the dispensers 10 are reusable and refillable.

FIGS. 22-23 show a linear feed magazine dispenser of the type shown in FIG. 9 that has been formed as a magazine group 120 and a dispenser head group 110. In this example, a neck is formed at the top of the magazine group 120 that mates to a dispensing volume internal to the dispenser head group 110. When the two groups 110, 120 are properly mated, they are held in place by, for example, mating snap lock elements 115, and create a reusable and refillable dispenser 10.

The magazine group 120 forms the magazine storage volume and feed path for testing supplies 66, 85 and contains the follower 25, and magazine spring 35. In some embodiments, feed regulators in the form of spring-loaded sears 90 are also located in the magazine group 120. The dispenser head group 110 includes the slide 45, ejector 55, and grip 50, or other mechanisms that may be adapted to deliver a single test strip 66 or lancet 85 from the dispenser 10.

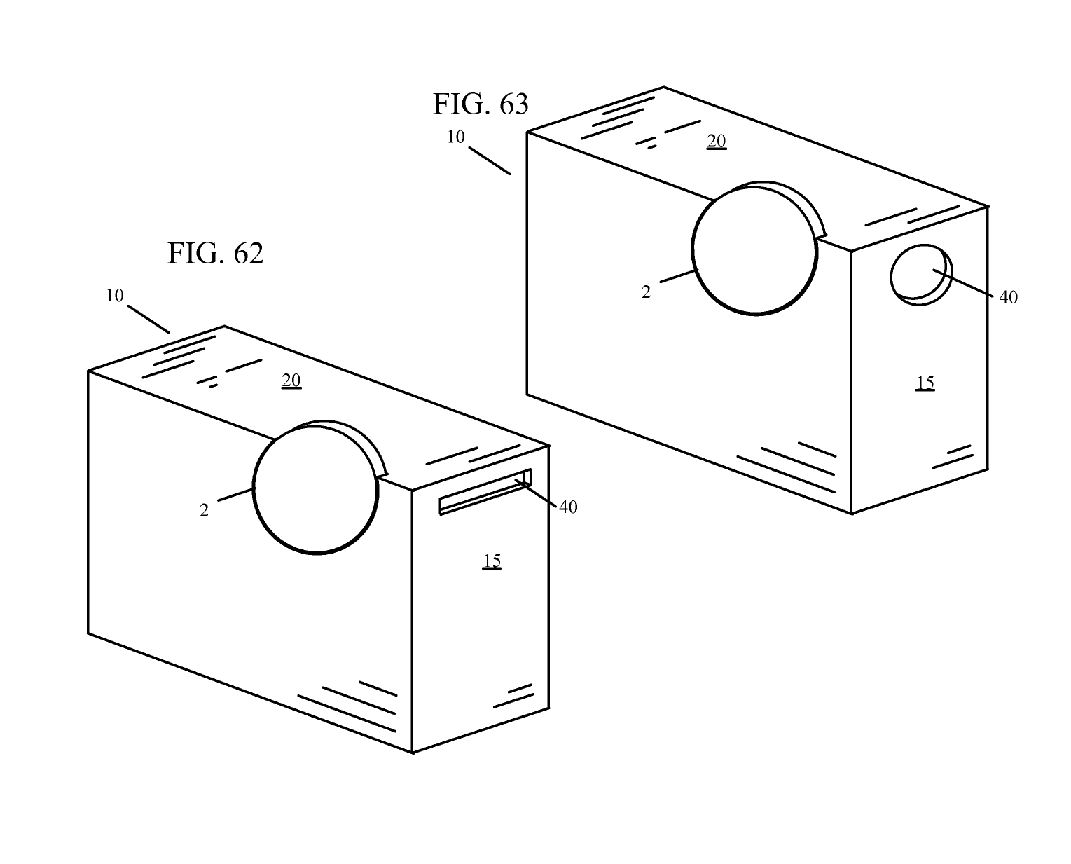

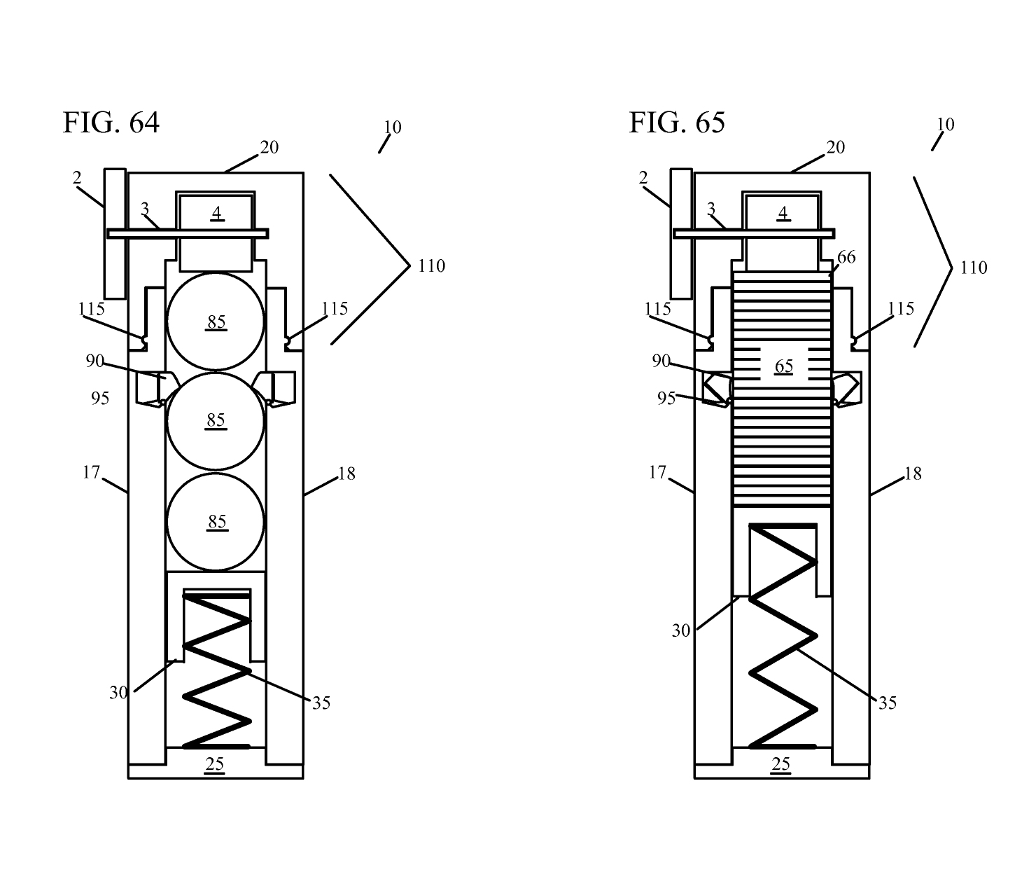

FIGS. 62-65 show a dispenser head group 110 with an alternative ejector mechanism. The mechanism is shown for both lancets 85 (FIGS. 63 and 64) and test strips 66 (FIGS. 62 and 65). In both embodiments, a drum-shaped roller ejector 4 is incorporated in the head group 110. The roller ejector 4 is substantially cylindrical in shape, but may be formed with a concave profile for example to better grip cylindrical lancet 85 bodies. The roller ejector 4 may be constructed of metal or hard plastic for example, and coated with a high tack rubber, or may be constructed from rubber or other resilient materials that have a high coefficient of friction relative to the materials from which the test strips 66 and lancet 85 bodies are constructed.

The roller ejector 4 is free to rotate on an axle 3 that extends out one side 17 of the dispenser head group 110. The axle 3 is in turn connected to a thumb wheel 2 actuator. The diameter of the thumb wheel 2 and the diameter of the roller ejector 4 may be chosen so that one rotation of the thumb wheel 2 will substantially eject one testing supply 66, 85 from the dispenser 10 through the port 40.

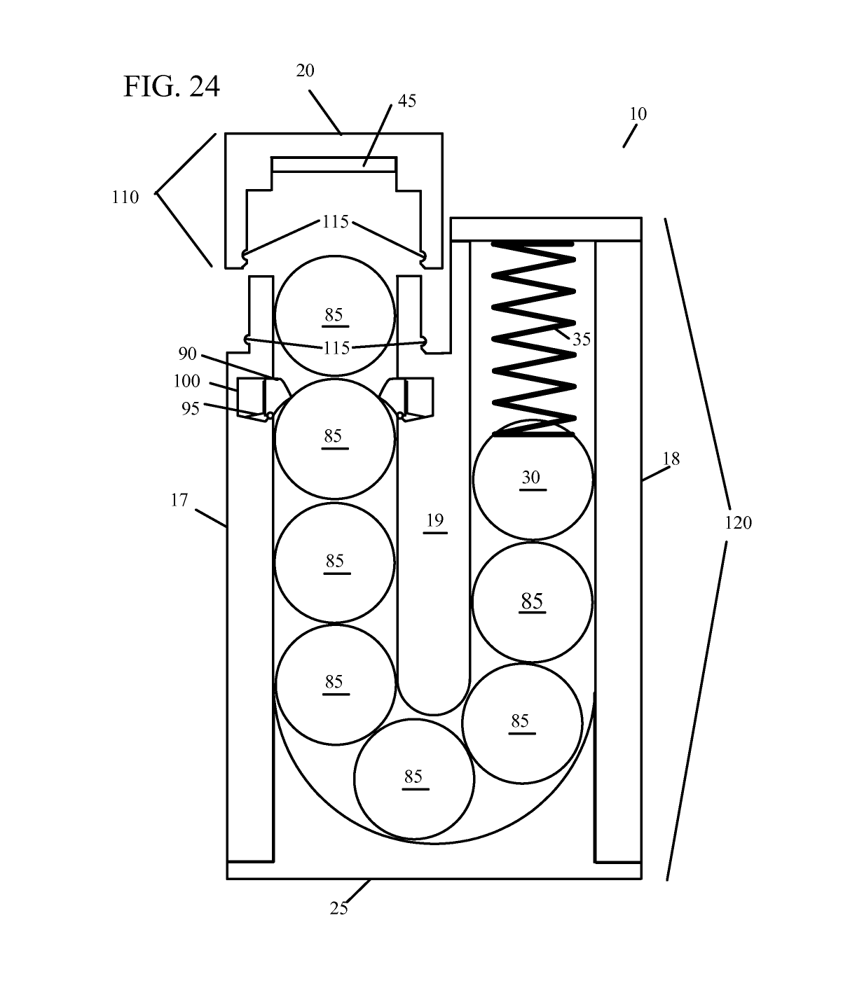

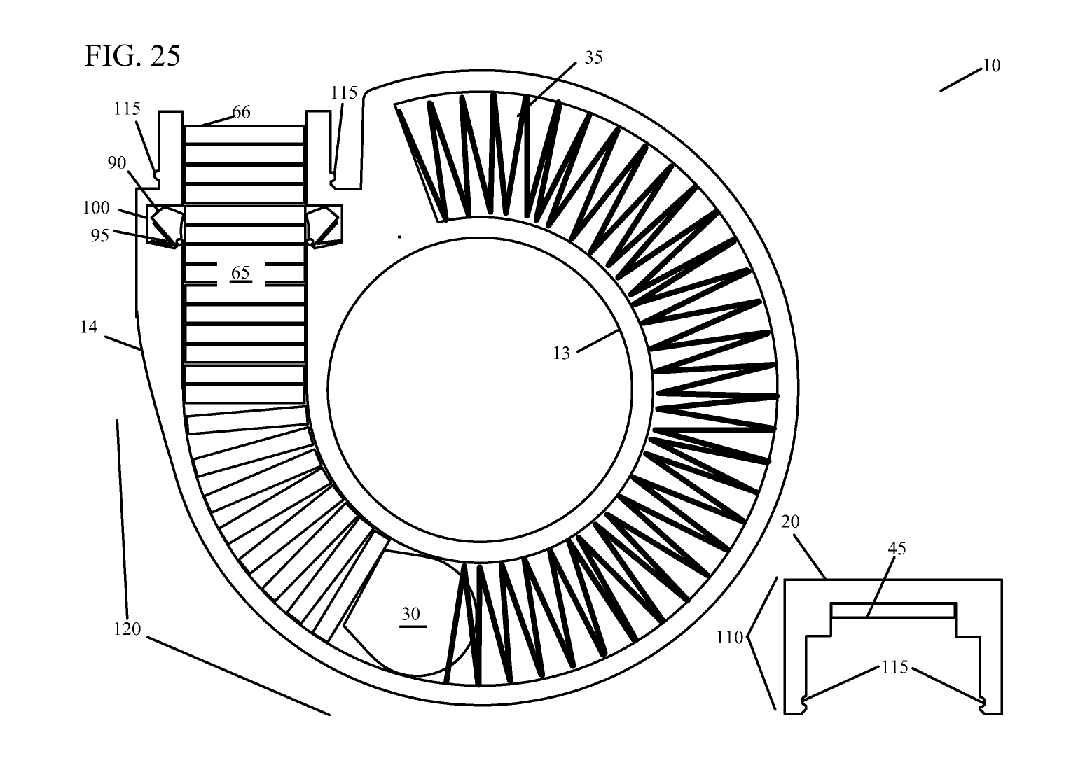

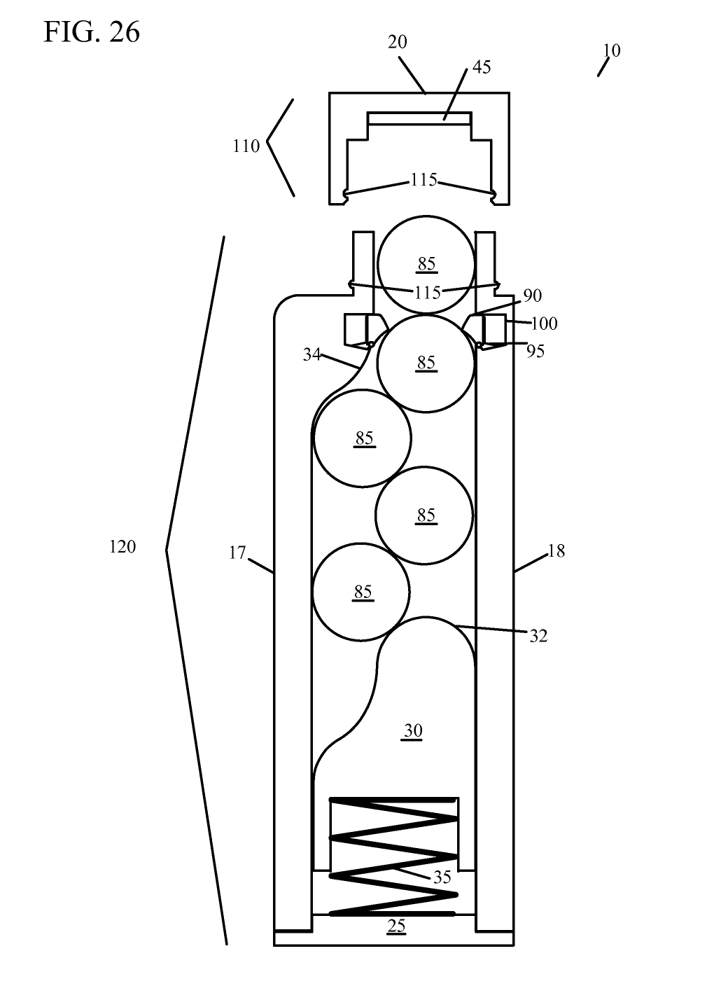

FIG. 24 shows a dispenser head group 110 and a magazine group 120 of a serpentine feed dispenser 10 (serpentine dispenser 10 also shown in FIG. 14). FIG. 25 shows the dispenser head group 110 with a magazine group 120 of a circular, spiral, or helical dispenser 10 (circular dispenser 10 also shown in FIGS. 15 and 16). FIG. 26 shows the magazine group 120 and dispenser head group 110 of a staggered stack dispenser 10 (staggered stack dispenser also shown in FIG. 13). While FIGS. 24 and 26 show a dispenser with lancets 85, and FIG. 25 shows a dispenser with test strips 66, any of the embodiments shown may be adapted to dispense lancets 85 or test strips 66. In each embodiment, the dispenser head group 110 may be universally adapted to a wide variety of magazine group 120 configurations. Although snap-fit elements 115 have been incorporated to illustrate embodiments that removably join the two groups 110, 120, other types of joints using hinges, fasteners, or other reusable connectors, are also possible.

Further, each of the embodiments shown in FIGS. 22-26 and FIGS. 62-65 may be constructed in tamper-resistant elements as well. Ampules 100, and/or a marking fluid reservoir 70 and applicator 75, may be incorporated, as previously described herein, in the dispensers 10 shown in FIGS. 22-26 and FIGS. 62-65. Also, to seal the unit, one need only apply an adhesive to the mating surfaces between the magazine group 120 and the dispenser head group 110 during assembly. Alternatively, the two groups 110, 120 may be joined after filling, and the joints fused with ultrasonic welding techniques.

Bulk loading of the reloadable dispenser embodiments shown in FIGS. 22-26 and FIGS. 62-65 may be performed rather than loading individual test strips 66 or lancets 85. Bulk loading saves time, is more hygienic, and requires much less tactile and motor involvement than loading individual test supplies 66, 85.

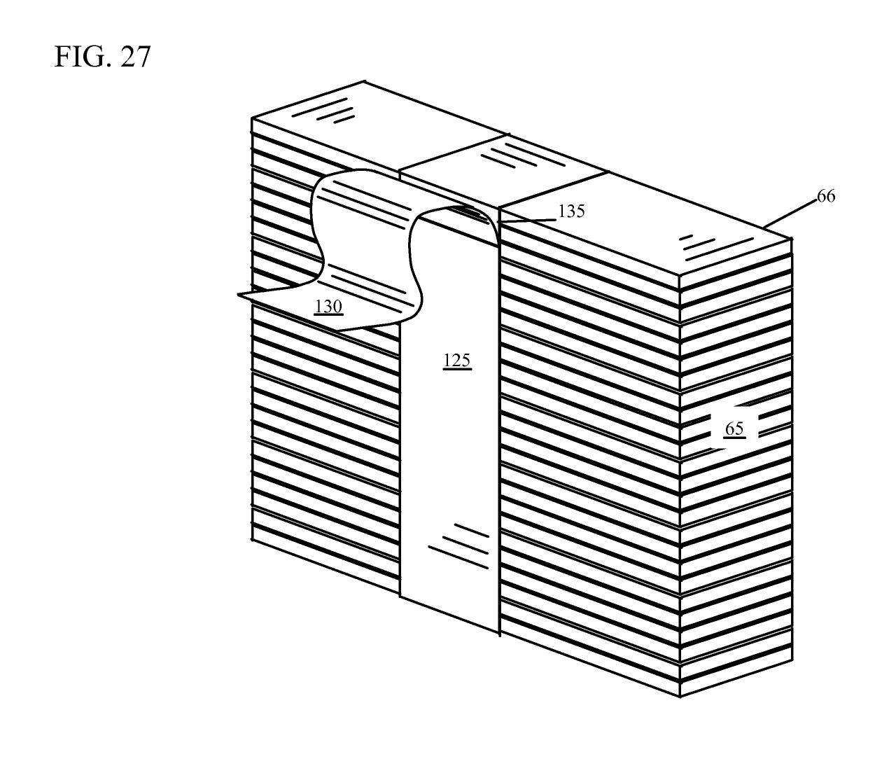

In one embodiment, shown in FIGS. 27-33, the magazine group 120 and the dispenser head group 110 are modified for bulk loading of a stack 65 of test strips 66. FIG. 27 shows the stack 65 of test strips forming a vertical column, with a band 125 binding the stack 65 together and maintaining the orientation of the strips 66 in the stack 65.

The band 125 may be made of an appropriate material, including but not limited to, paper, film backed paper, or thin plastic films such as Mylar® or linear low-density polyethylene (LLDP). LLDP may be particularly useful in binding stacks 65 that are to be loaded into magazine groups 120 that have circular, spiral, or helical feed pathways. LLDP will tightly bind the stack, but will also stretch enough to allow the stack to bend around curves in such magazines. The banded stack may be inexpensively packaged, for example, in foil or thin film wrappings. The band 125 is preferably wrapped around the stack 65 near its center, and one end of the band 125 is removably adhered to near the other end of the band 125 at a location 135 near the top of the stack. A pull strip leader 130 preferably extends freely from the adhesion location 135 to facilitate removal of the band 125 after loading.

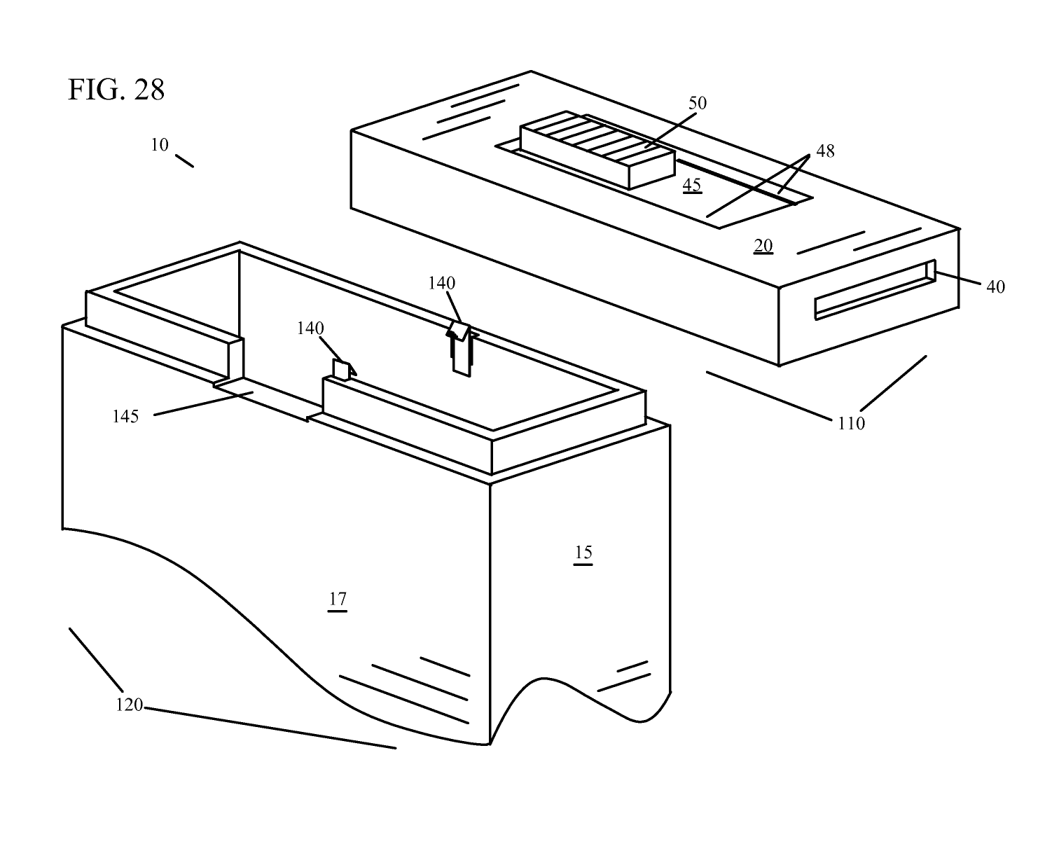

FIG. 28 shows a magazine group 120 that accepts the bulk loading test strip stack 65 of FIG. 27. Feed regulators in the form of barbed sears 140 are added on two interior sides of the dispenser 10 that form the magazine and feed path. A “sear” or “sears”, as used herein, generally refer to any structural element that allows motion of another element in one direction, but prevents the motion of that same element in the opposite direction until such time as the sear is actively prevented from blocking such motion. Alternatively a sear may allow bilateral motion of an element until it is actively enabled to stop motion in one direction (as is the case with the spring-loaded sears 90 of FIGS. 9-11) In some embodiments, a sear is a strip (for example of metal, plastic, or other material) with two ends. One end of the strip forms, for example, an angled or curved hook or barb. As another element moves past the angle or curve, it applies lateral force to the sear. The strip forming the sear may be flexible, or hinged at its opposite end, to allow the sear to move in response to this applied force and allow the moving element to pass. Once past the sear, the hook or barb engages a portion of the moving element to prevent its movement in the opposite direction until the hook or barb is moved out of engagement with the moving element.

In addition, a notch 145 is located on one side 17 of the magazine group 120. When the bulk loading test strip stack 65 is fully inserted, the pull strip leader 130 may be positioned in the notch 145 and remains free to slide out of the dispenser 10 after the dispenser head group 110 has been attached. Once loaded and reassembled, the band 125 may be pulled out of the dispenser 10, freeing the individual test strips 66 in the stack 65 for dispensing.

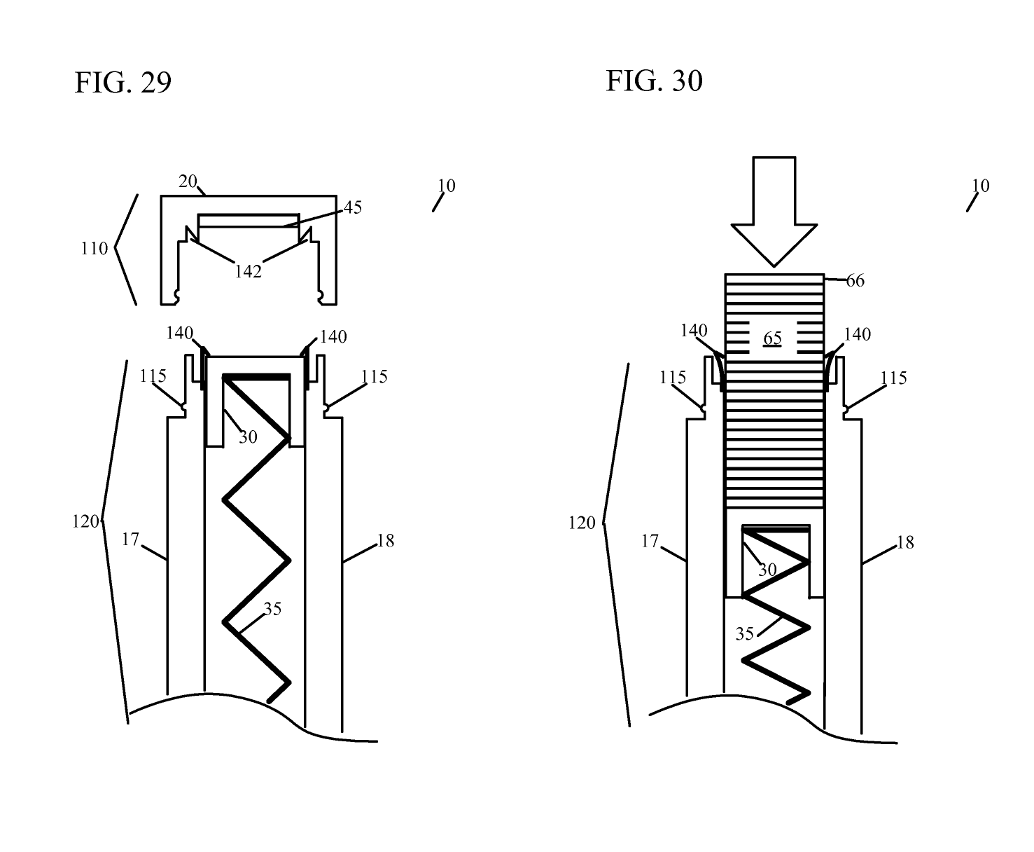

FIG. 29 shows a cross section of the magazine group 120 and the dispenser head group 110 at the location of the barbed sears 140. The barbed sears 140 preferably have a barb, hook, or similar protrusion extending into the magazine volume, and are angled at their top. The barbed sears 140 are constructed of a material that provides them with a resilient spring action, including but not limited to, spring steel, or plastic as part of the dispenser molding. When the dispenser 10 is empty, the barbed sears 140 serve to retain the follower 30 within the dispenser 10 storage volume.

The inside of the dispenser head group 110 has also been prepared with sear blocks 142 that push the barbed sears 140 out of the magazine feed pathway when the dispenser head group 110 and the magazine group 120 are fully mated together.

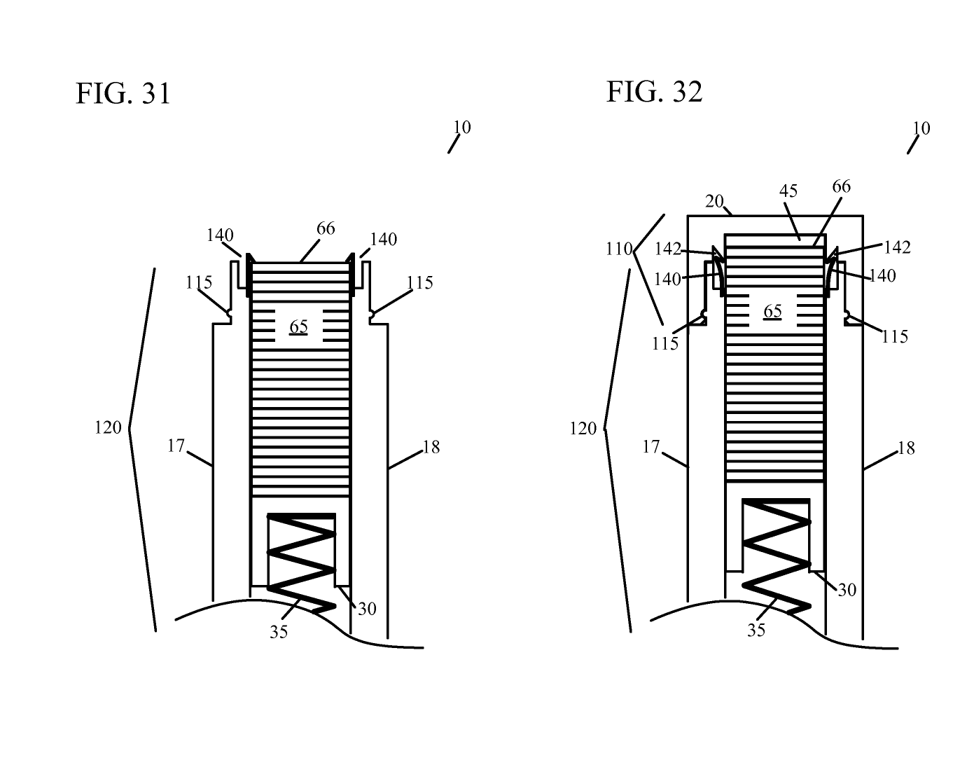

When the banded stack 65 of test strips 66 is inserted into the magazine group 120, shown in FIG. 30, the barbed sears 140 are bent outward and allow the stack 65 to freely slide downward past them. Once the top-most test strip 66 moves past the barbed sears 140, the barbed sears 140 spring back into the magazine feed path and block the stack 65 of test strips 66 from being pushed back out of the magazine group 120 by the magazine spring 35, as shown in FIG. 31.

When the dispenser head group 110 is fixed in place on the magazine group 120, the sear blocks 142 engage the barbed sears 140 and bend them back out of the magazine feed pathway, as shown in FIG. 32. The magazine feed spring 30 is then free to push the stack 65 upward against the top 20 and slide 45 so that the top-most test strip 66 is ready to be dispensed.

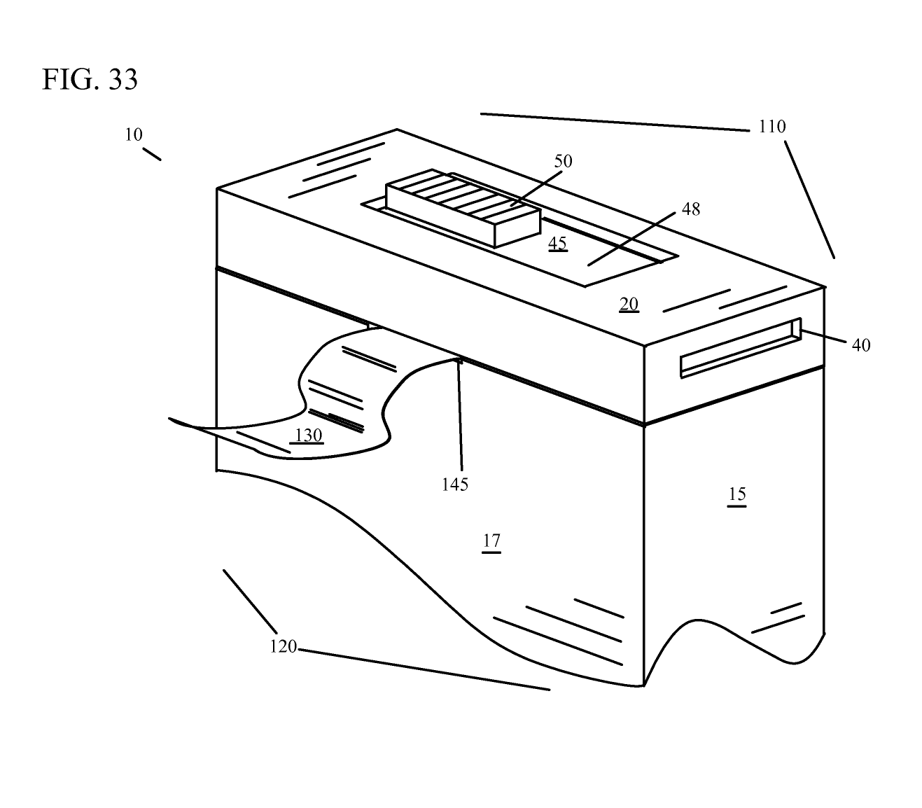

When bulk loading is complete and the dispenser 10 is reassembled (FIG. 33), the band pull leader 130 is pulled to separate the adhesion location 135. The entire band 125 is then free to be pulled out of the dispenser 10 through the notch 145, and the dispenser is ready for use.

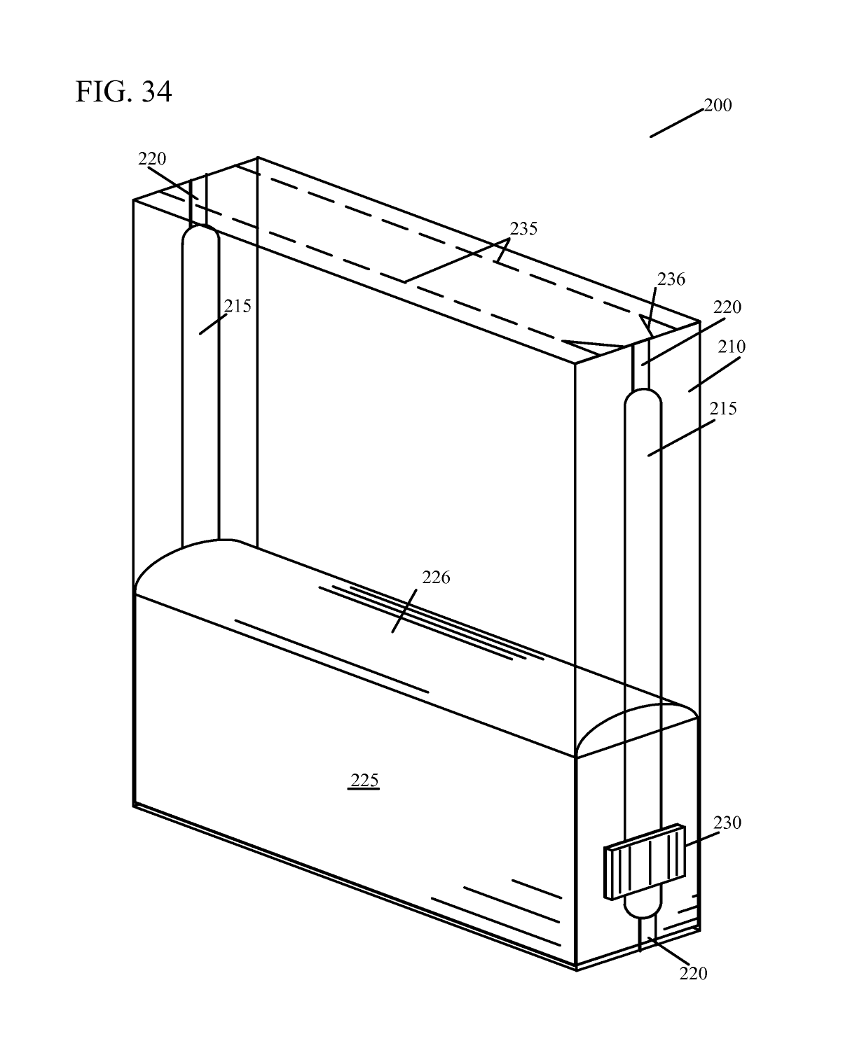

In an alternative embodiment, test strips 66 and lancets 85 are prepared in bulk loaders 200 shown in FIGS. 34-36. A bulk loader 200, shown in FIG. 34, is generally formed by a rectangular tube 210. The tube 210 may be constructed from a blank pattern die cut from a sheet of thin card stock, or as shown in the FIG. 34, transparent plastic sheet, and sealed 220 on two sides. One end of the tube 210 is closed and prepared with a perforated 235 pull strip 236 that removes the end of the tube 210 when it is ready for use. Alternatively, a removable cap may seal the open end of the tube 210.

Once filled with testing supplies 66, 85, a pusher 225 is provided at the other end of the tube 210, and may slide toward the opposite, open, end of the tube 210, pushing the testing supplies 66, 85, contained therein out of the bulk dispenser 200. The top side 226 of the pusher 225 is formed as an arch, semi-circle, or other shape that will push the testing supplies 66, 85, but not interfere with other mechanisms in the magazine group 120 being loaded, as will be discussed further below.

The pusher 225 is also provided with a handle 230 on each end that extends out of the tube 210, and moves with the pusher through slots 215 provided in each side of the tube 210. The pusher 225 may be formed by injection molding, vacuum molding, blow molding, or from a card stock blank folded and glued to form the structure of the pusher 225.

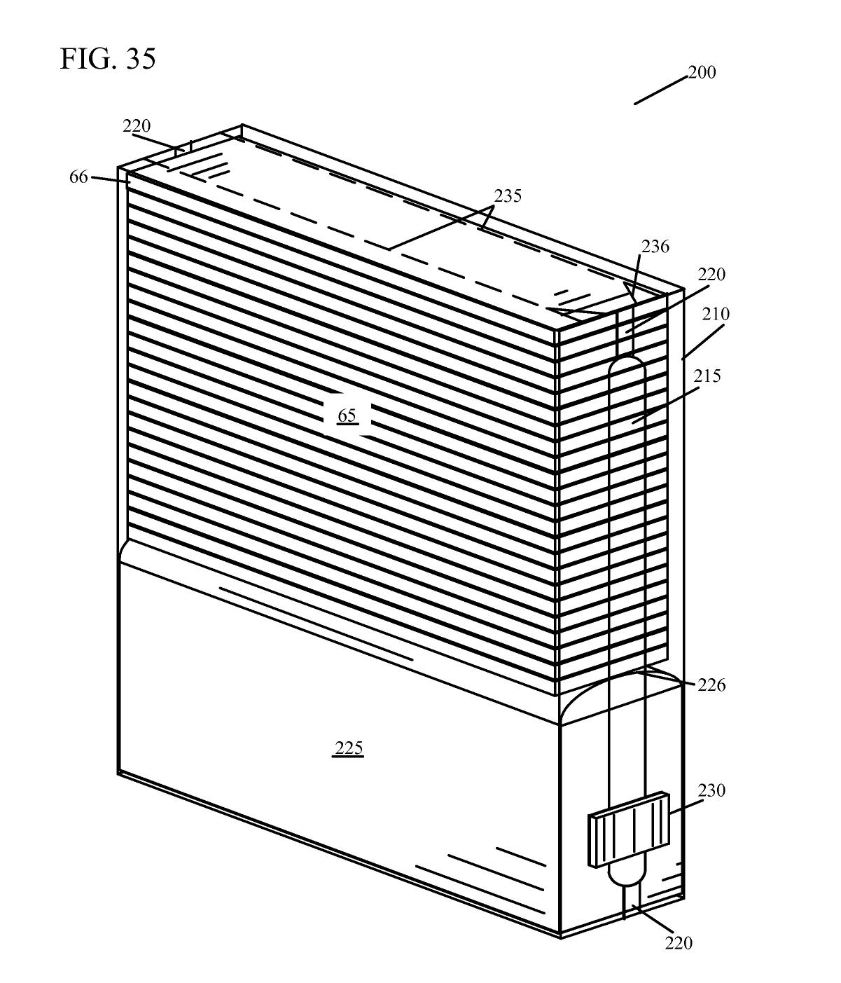

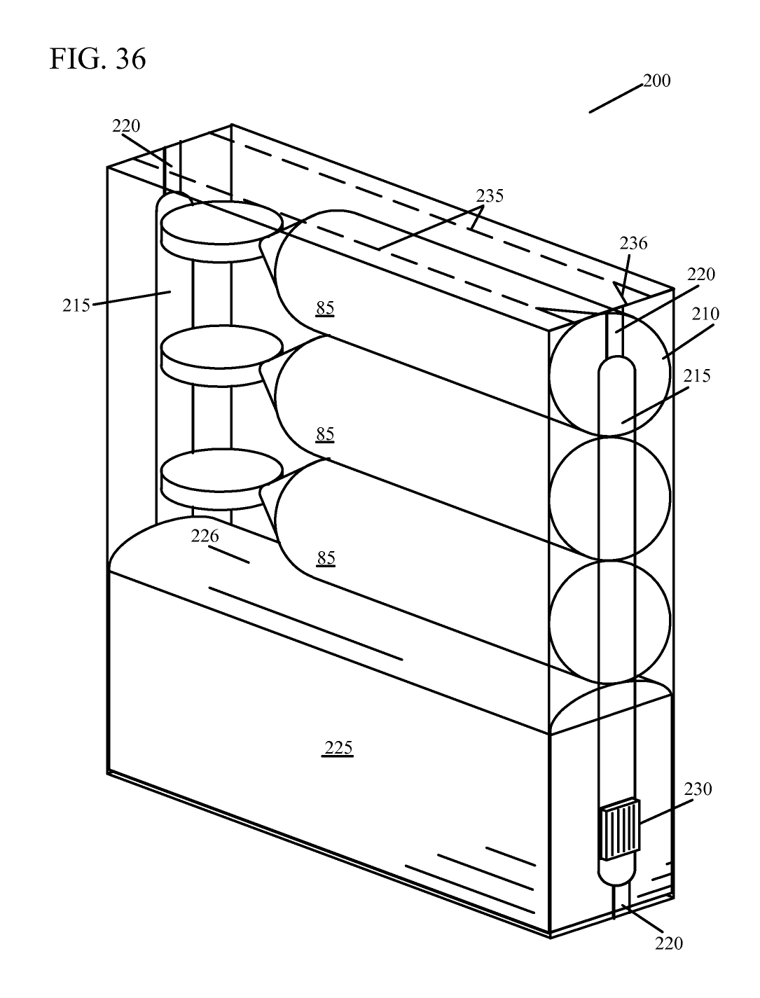

In operation, the user removes the pull strip 236 to open the bulk loader 200, and inserts the open end into the dispenser magazine group 120. The pusher 225 is then moved toward the open end of the bulk loader 200 using the handles 230, causing the testing supplies 66, 85, within the bulk loader 120 to move into the dispenser magazine group 120. FIG. 35 shows a bulk loader 200 filled with a stack 65 of test strips 66. FIG. 36 shows a bulk loader 200 filled with a stack of lancets 85. Although a limited number of test strips 66 and lancets 85 are shown in FIGS. 35-36, the bulk loader 200 may be constructed in any length to accommodate any desired number of testing supplies 66, 85. FIG. 36 also shows an alternative embodiment of a pusher handle 230 that only protrudes through the tube 210 slots 215, but does not otherwise surround the tube 210 material.

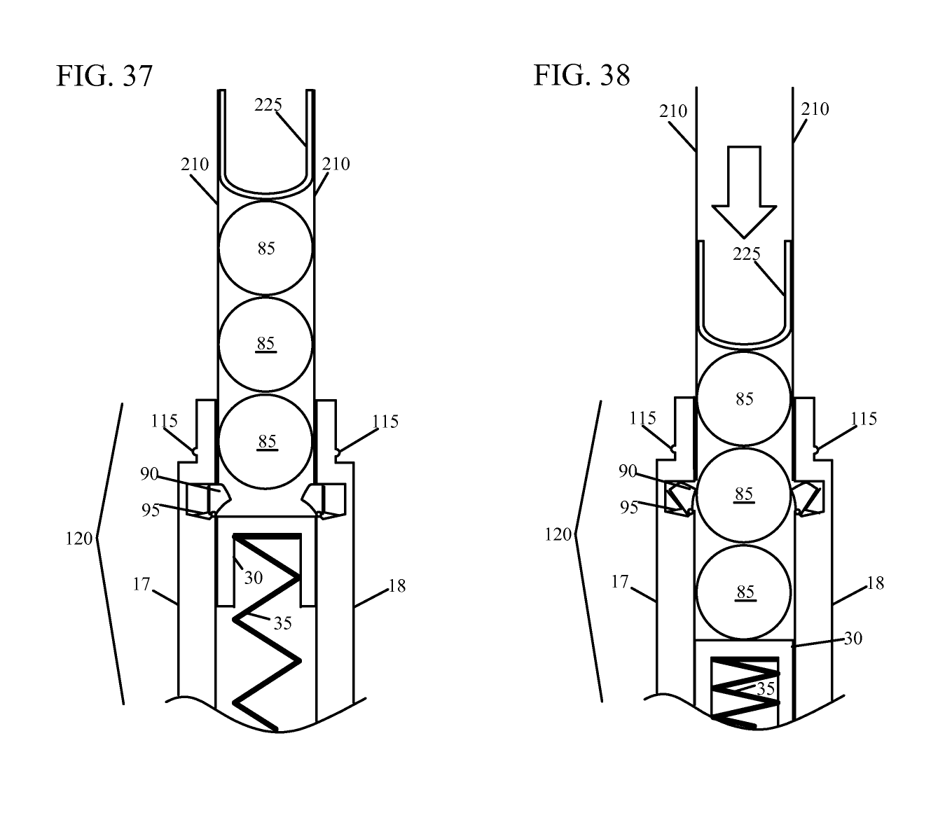

FIGS. 37-42 illustrate the operation of the bulk loader 200 filling a magazine group 120 with a number of lancets 85. The open end of the bulk loader 200 tube 210 is inserted into the open end of the magazine group 120 after removal of the dispenser head group 110. The tube 210 is inserted to the level of the spring-loaded sears 90. As shown in FIG. 38, the pusher 225 is moved toward the magazine group 120, which in turn pushes the lancets 85 into the magazine group against the follower 30, compressing the magazine spring 35.

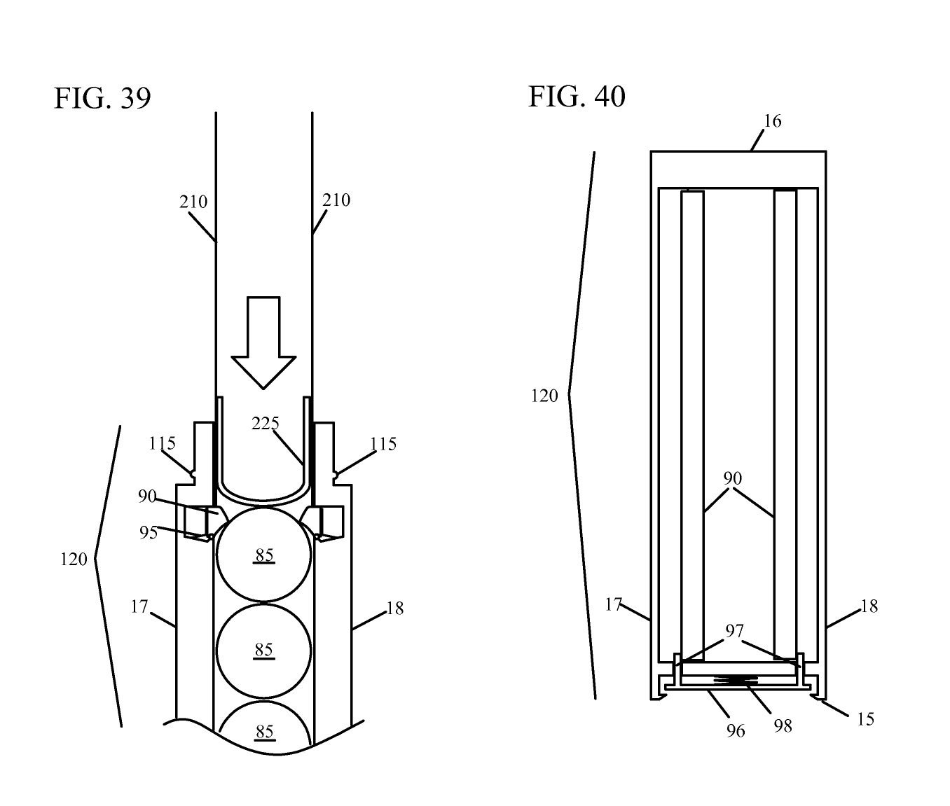

As the lancets 85 move into the magazine group 120, they push the spring-loaded sears 90 out of the feed path so the lancets 85 may move freely past. The length of the slots 215 in the bulk loader 200, and position of the pusher handles 230, limits the maximum travel of the pusher 225 in the bulk loader 200 tube 210. As shown in FIG. 39, when the pusher 225 is at its most extreme position at the open end of the bulk loader 200 tube 210, the last lancet 85 from the bulk loader 200 is positioned below the spring-loaded sears 90, which are now free to extend into the feed pathway under pressure from the springs 95.

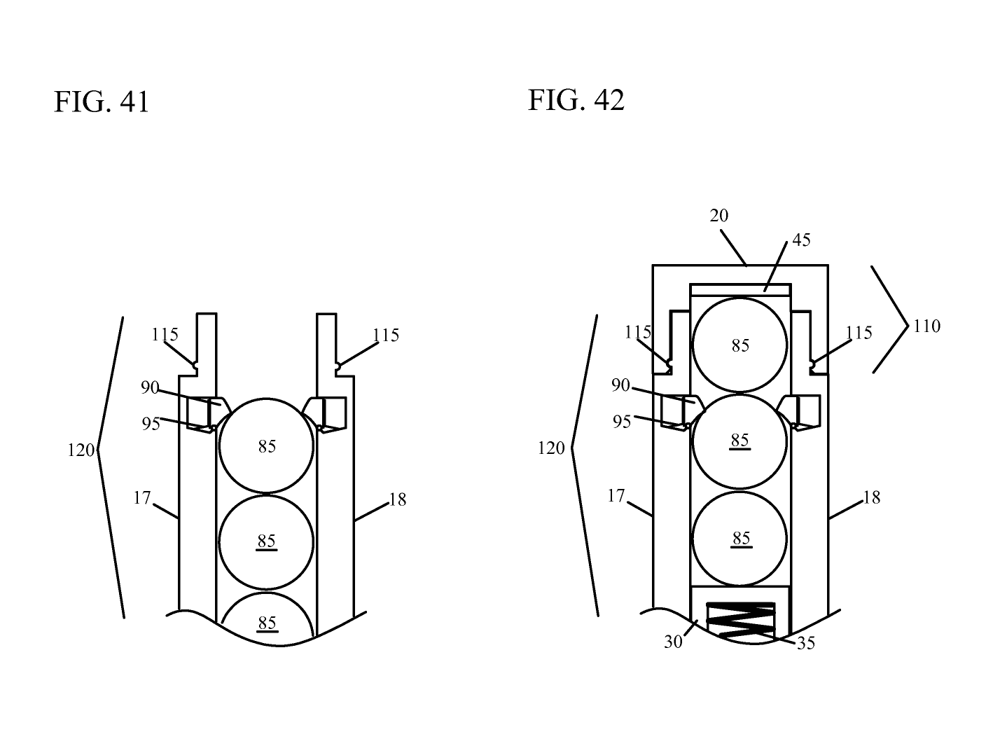

The user now depresses a feed lock 96, shown in FIG. 40, which prevents the spring-loaded sears 90 from moving out of the feed path by locating pins 97. As shown in FIG. 41, the bulk loader 200 can now be removed from the magazine group 120, while the spring-loaded sears 90 retain the lancets 85 inside the magazine group.

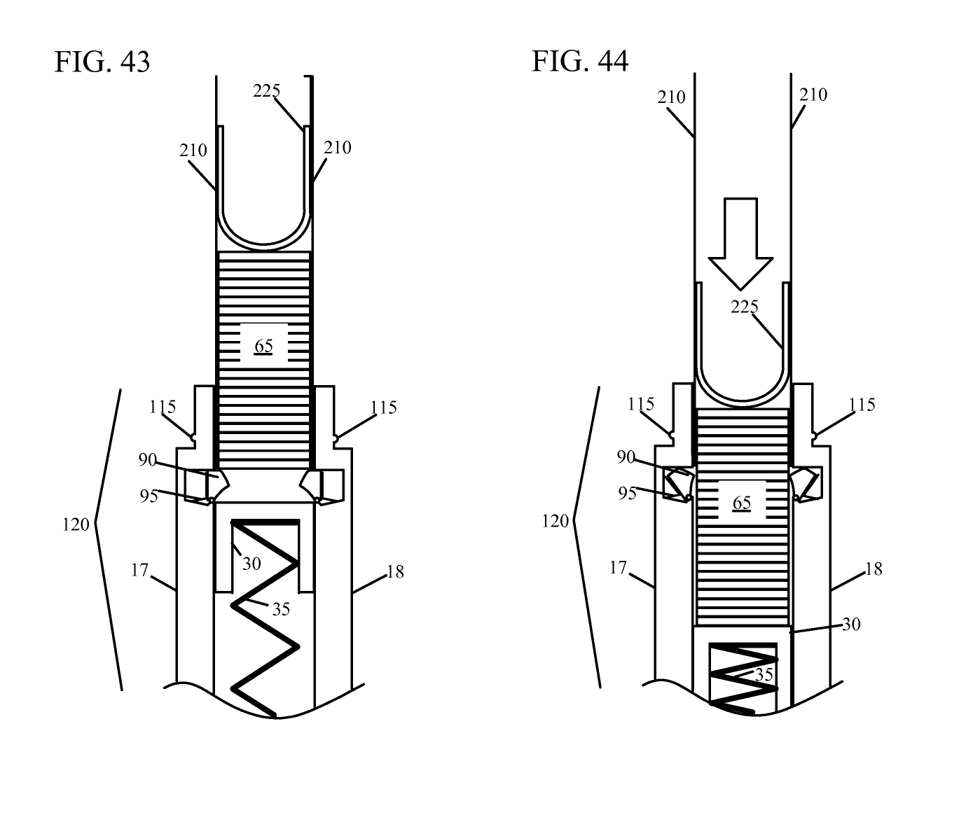

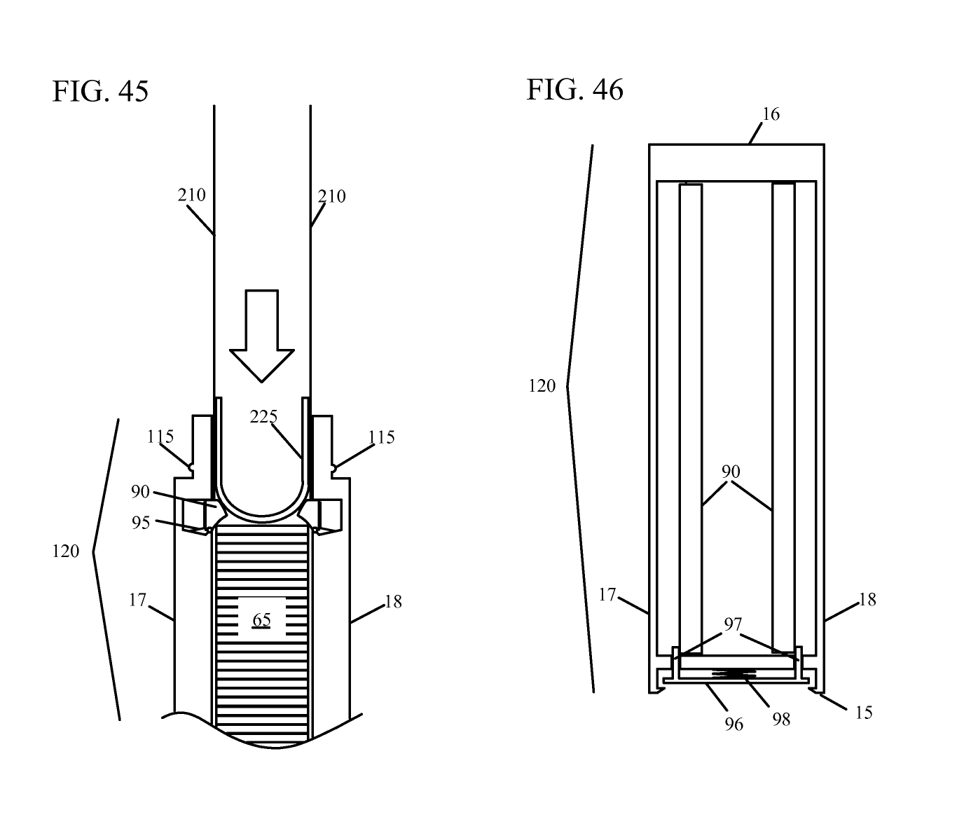

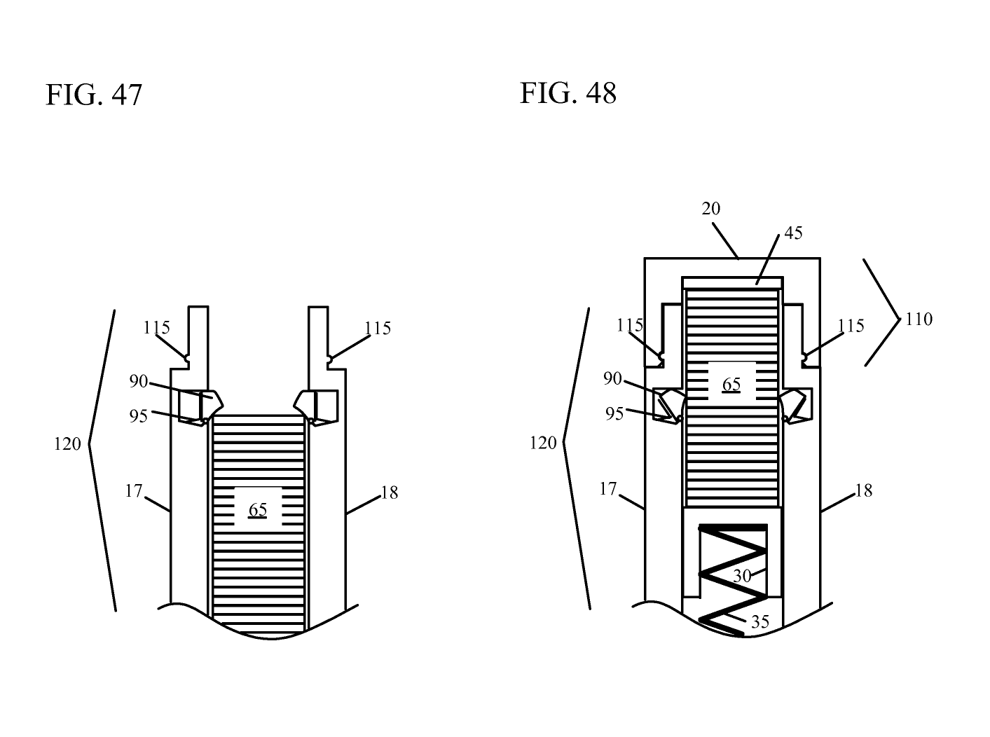

After replacement of the dispenser head group 110 on the magazine group 120, as shown in FIG. 42, the feed lock 96 is released, for example under pressure of a return spring 98 (shown in FIG. 40), and the pins 97 are withdrawn, allowing the spring-loaded sears 90 to move out of the magazine feed pathway. Pressure of the magazine spring 35 on the follower 30 and loaded lancets 85 then pushes a lancet 85 into a ready position for dispensing. FIGS. 43-48 illustrate substantially the same procedure when a stack 65 of test strips are being bulk loaded using the bulk loader 200 of FIG. 35. Although spring loaded sears 90 are shown in FIGS. 43-48, barbed sears 140 shown in FIGS. 28-32 may also be used with test strips, and appropriate cutouts and indentations provided in the bulk loader tube 210 and pusher 225 so that the bulk loader tube 210 may be inserted past the barbed sears 140, and the pusher 225 may push a test strip stack 65 past the barbed sears 140.

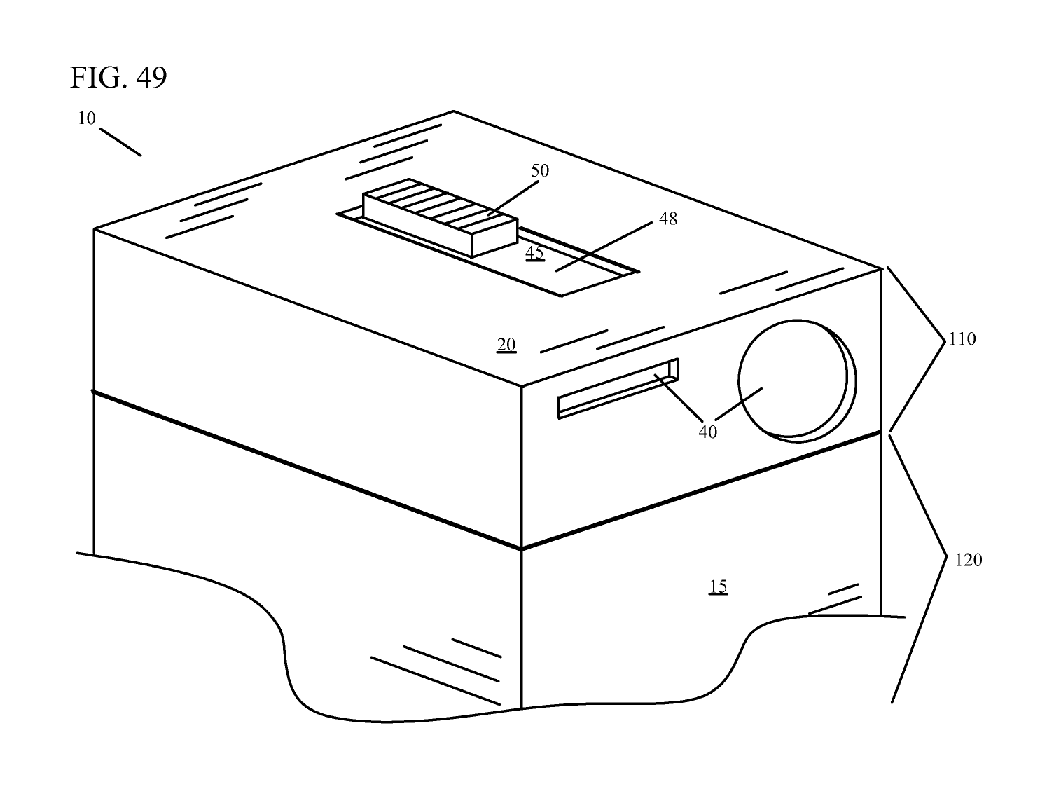

Dispensers 10 that dispense both test strips 66 and lancets 85 encourage patients to purchase, and use, one lancet 85 and one test strip 66 for each blood test. FIG. 49 shows a perspective of a dispenser 10 with a widened magazine group 120 to accommodate two internal magazine volumes, followers 30, and magazine springs 35. Similarly, the dispenser 10 head group 110 has also been modified with a single grip 50 and slide 45 actuating an ejector in each magazine volume.

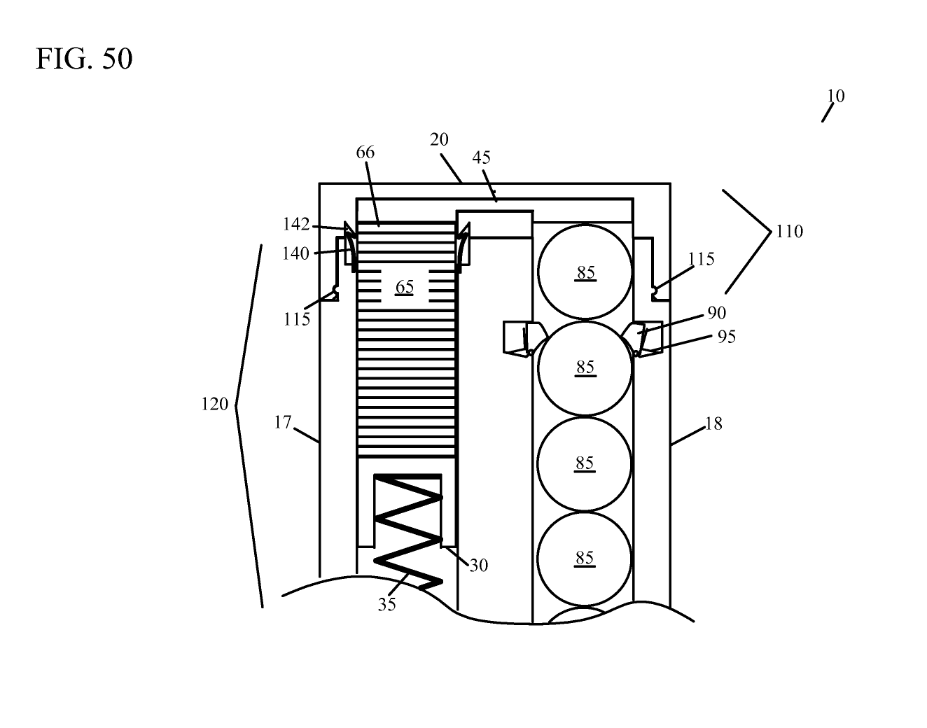

FIG. 50 shows a cross-section of this arrangement in which the single slide 45 spans a first magazine holding a stack of test strips 65, and a second magazine holding lancets 85. The ejector 55 on the test strip (not shown in this figure) side is dimensioned to move a single test strip 66, and a second ejector attached to the same slide 45 on the lancet 85 side is dimensioned to move a single lancet 85. Thus, with one movement of the actuator grip 50 and slide 45, a single test strip 66 and a single lancet 85 are dispensed through the dispenser ports 40 at the same time. Alternatively, the test strip magazine and the lancet magazine each have a separate, dedicated actuator grip 50, slide 45 and ejector 55, which allows individual dispensing from each of the magazine storage volumes.

The dual dispenser 10 embodiments shown in FIGS. 49-50 may be constructed as sealed single use dispensers 10, and may also be equipped with any of the tamper-resistant elements shown in FIGS. 6, 8, and 17-21. In other embodiments, dual dispenser embodiments of FIGS. 49-50 may be configured for reuse and bulk loading using the various elements shown in FIGS. 27-48.

Further, dual dispenser 10 embodiments may be constructed with any geometrically compatible combination of magazine groups 120, including those previously described herein. In one embodiment, two linear feed magazines may be formed side-by-side. In another embodiment, as lancets 85 require more space than test strips 66, a staggered, serpentine, circular, spiral, or helical magazine group 120 for lancets 85 may be combined with a linear magazine group for test strips 66.

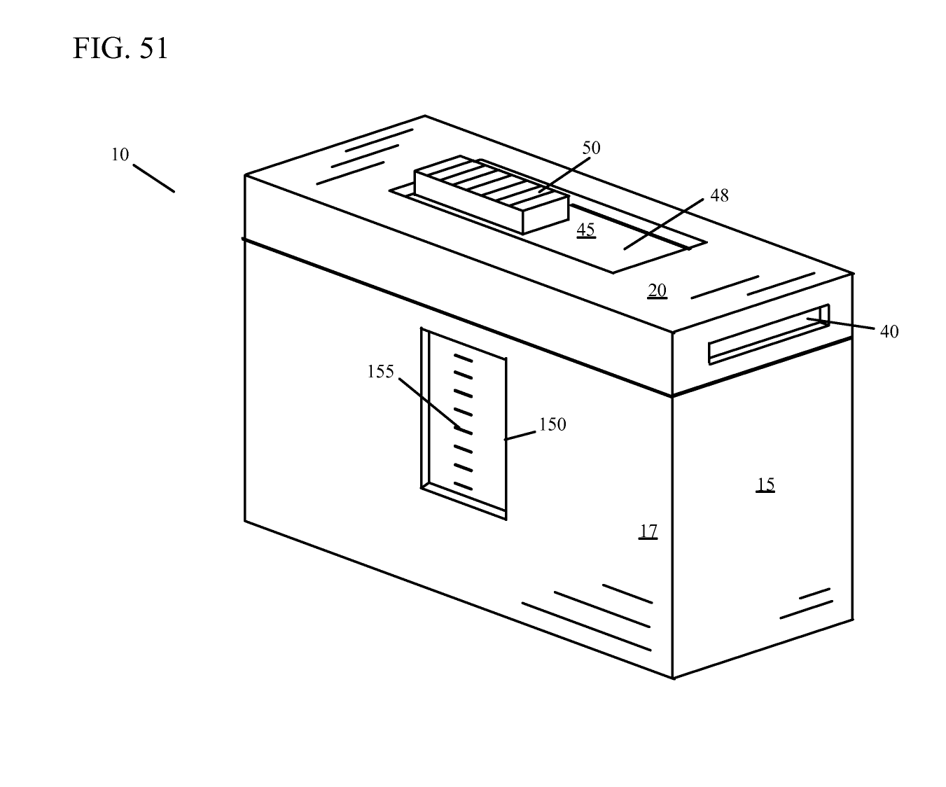

In some embodiments, one or more sides of the magazine group 120 may be provided with a transparent viewing window 150 that is graduated 155 according to the number of testing supplies 66, 85 remaining in the dispenser 10, as shown in FIG. 51. For example, a visible line on the follower 30 may align with each successive graduation 155 as the follower 30 moves to different positions when individual testing supplies 66, 85 are dispensed. While FIG. 51 shows a viewing window 150 in use with a linear box magazine group 120, the viewing window 150 may also be incorporated into any of the dispenser 10 shapes described previously herein, whether they be of a sealed, tamper-resistant configuration, or a refillable, reusable configuration.

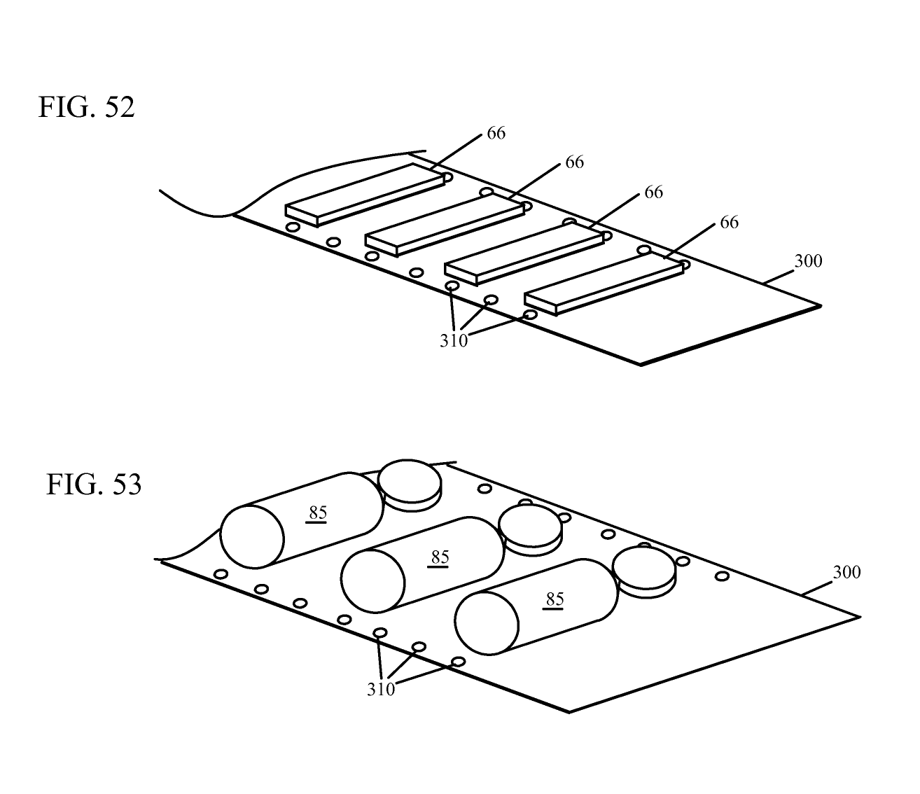

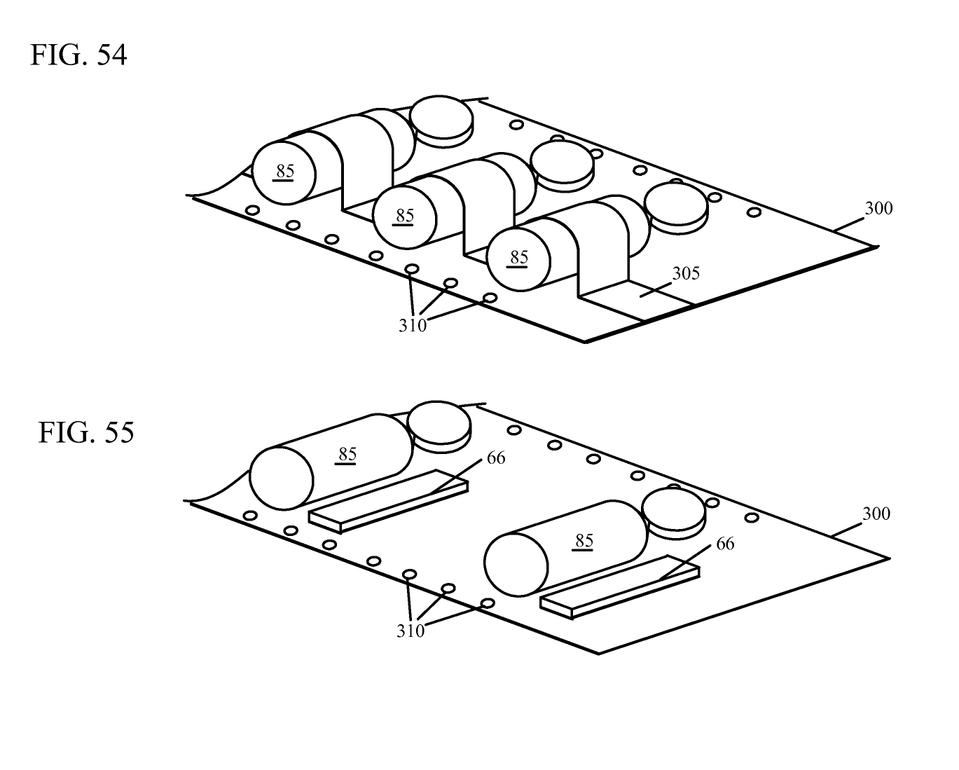

In other embodiments, shown in FIGS. 52-55, testing supplies 66, 85 are prepared on carrier tapes 300. Test strips 66 may be removably affixed to the carrier tape 300. Lancets 85 may also be removably affixed to carrier tapes 300, as shown in FIG. 53. Alternatively, as shown in FIG. 54, lancets 85 may be affixed to the carrier tape 300 using a secondary tape 305, forming a bandolier. In other embodiments, shown in FIG. 55, lancets 85 and test strips 66 may be applied in working pairs on the carrier tape 300, and affixed with removable adhesive, a secondary tape 305 as shown in FIG. 54, or a combination of both. The carrier tapes 300 may be constructed of any appropriate material, including but not limited to, paper, coated paper, or thin plastic films. In some embodiments, perforations 310 are provided at regular intervals along each edge of the carrier tape 300.

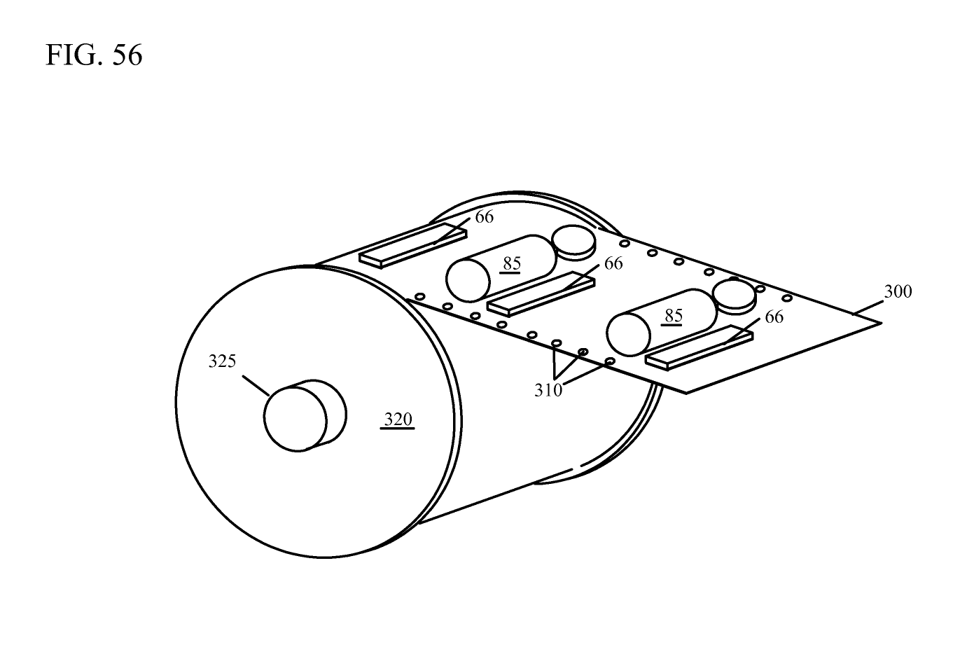

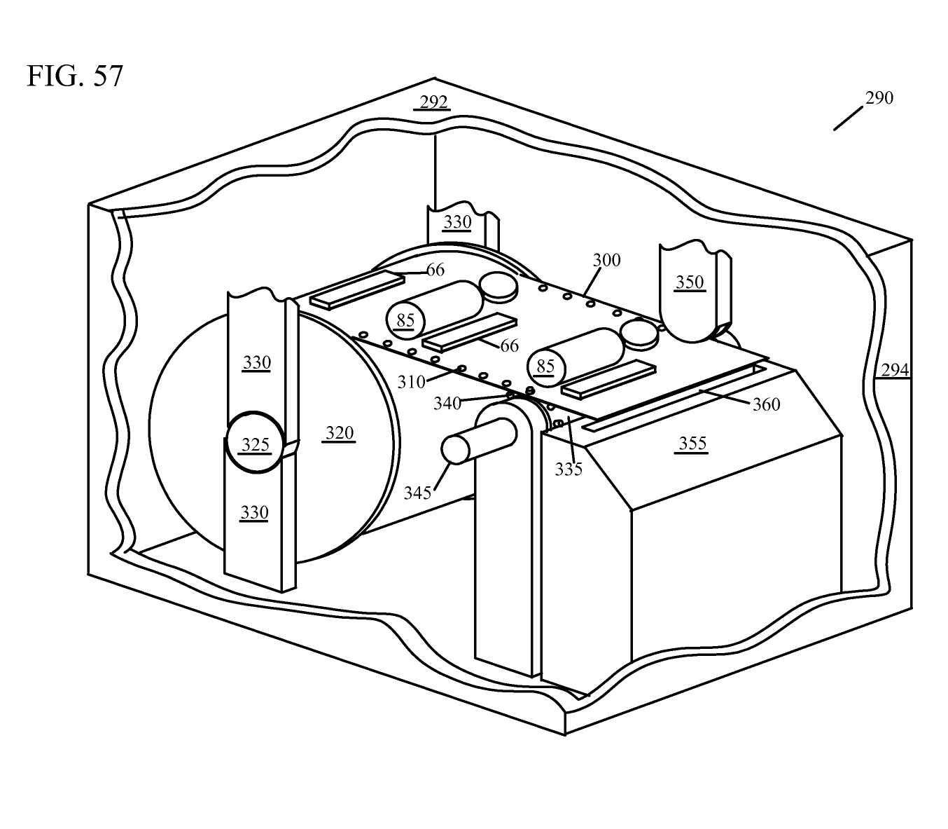

Referring now to FIG. 56, any of the carrier tapes 300 shown in FIGS. 52-55 may be wound around a spool 320 with an axle 325. The spool 320 is dimensioned to accommodate a number of windings of a carrier tape 300 carrying a given number of testing supplies 66, 85. Although testing supplies 66, 85, are affixed to the majority of the length of the carrier tape 300, in this figure, testing supplies 66, 85 are only shown on the unwound end of the carrier tape 300 for clarity. Also, although working pairs of test strips 66 and lancets 85 are shown in groups, the carrier tape 300 may also carry evenly spaced lancets 85 only, or alternatively, evenly spaced test strip 66 only.

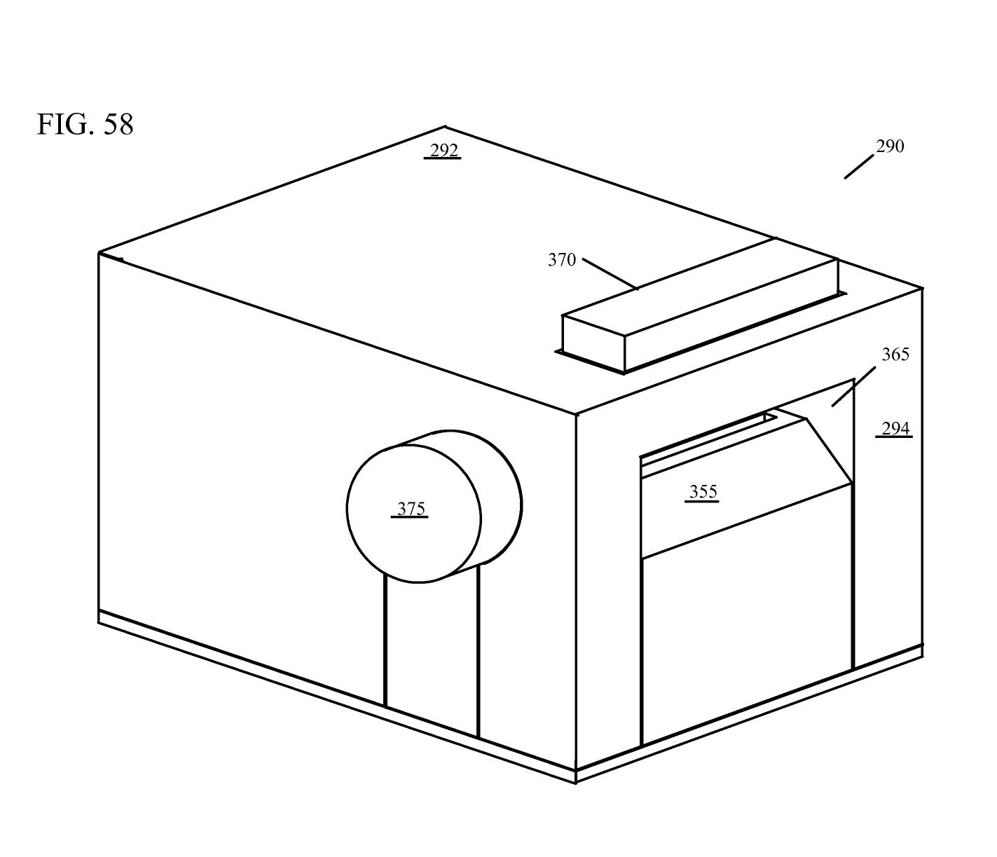

FIG. 57 shows a partial cut-away perspective of a dispenser 290 that uses the loaded spool 320 of FIG. 56. The spool 320 is held in the dispenser 290 by supports 330 on either end, and from above and below, so that it is free to rotate. The carrier tape 300 is fed over a drive drum 335 that has a series of pins 340 extending radially from its surface at each end. The pins 340 are spaced such that, when the drive drum 335 rotates, the pins 340 engage the perforations 310 along both sides of the carrier tape 300. Thus, as the drive drum 335 rotates, the carrier tape 300 is drawn off the spool 320 toward an exit port 365 (FIG. 58) in the front 294 of the dispenser 290. A guide 350 is provided on each side of the carrier tape 300 (only one is shown in FIG. 57) to maintain contact between the carrier tape 300 and the drive drum 335, and to ensure proper engagement of the perforations 310 and pins 340.

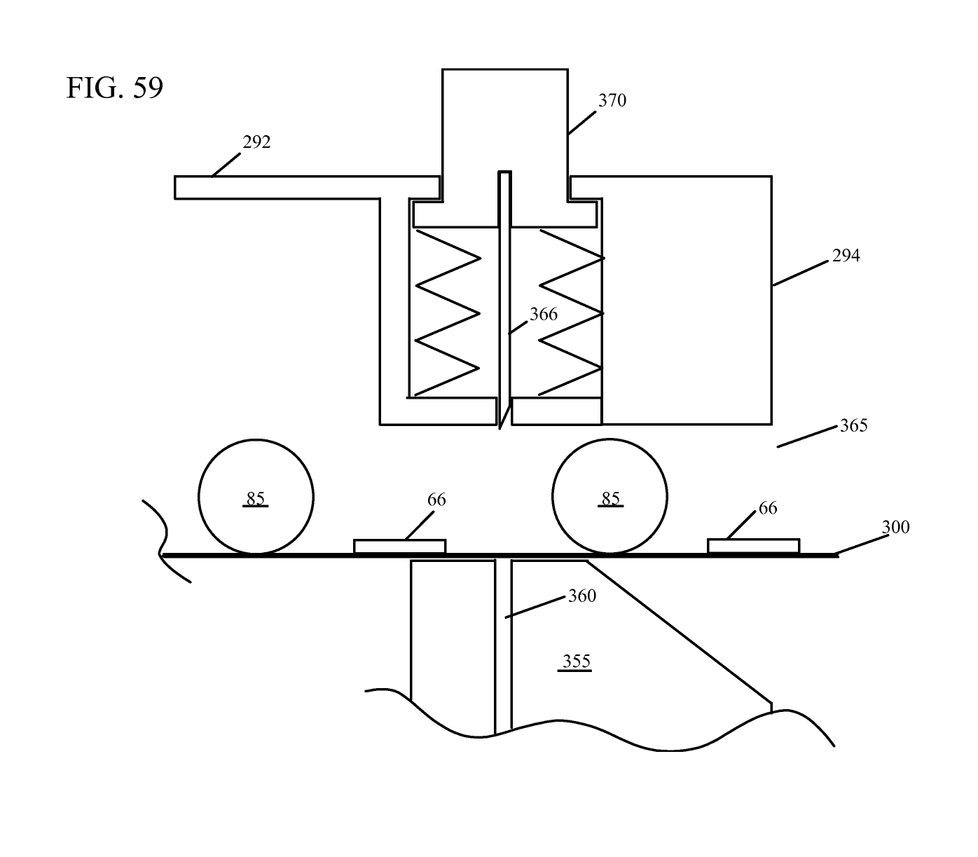

The bottom of the exit port 365 is formed by an anvil 355 having a shear slot 360, over which the carrier strip 300 moves toward the exit port 360. As shown in FIGS. 58-59, a blade 366 is provided in the top 292 of the dispenser 290 to cut the carrier strip 300 after it has been moved a fixed increment past the shear slot 360. The blade 366 may be actuated by pressing a cutter actuator 370 on the top 292 of the dispenser 290.

The carrier tape 300 movement increment, as shown in FIG. 58, may be controlled by a dispensing actuator knob 375 that is calibrated to move the carrier tape 300 past the shear slot one test supply 66, 85 (or working pair 66 and 85) per rotation. A spring-loaded detent on the drive drum 355 axle 345 (FIG. 57), extending out of the dispenser indicates when a single rotation has been completed, and the dispensed testing supplies may be freed from the rest of the carrier tape 300 by cutting the carrier tape 300 with the blade 366. Alternatively, a detent may be incorporated into the dispensing actuator knob 375 to indicate when an appropriate increment of the carrier tape 300 has occurred.

The dispensers 290 shown in FIGS. 52-59 may be constructed as a single use, disposable dispenser 290, or a reusable, refillable dispenser 290. In single use embodiments, the tamper-resistant ampules 100 shown in FIGS. 17-21 may be incorporated at various locations within the dispenser 290 construction to mark and or disable testing supplies if attempts to open the dispenser 290 are made. Further, in some embodiments, the marking reservoir 70 and applicator 75 of FIG. 6 may also, or alternatively, be incorporated to prevent intentional emptying of the dispenser 290 and unauthorized resale of its contents.

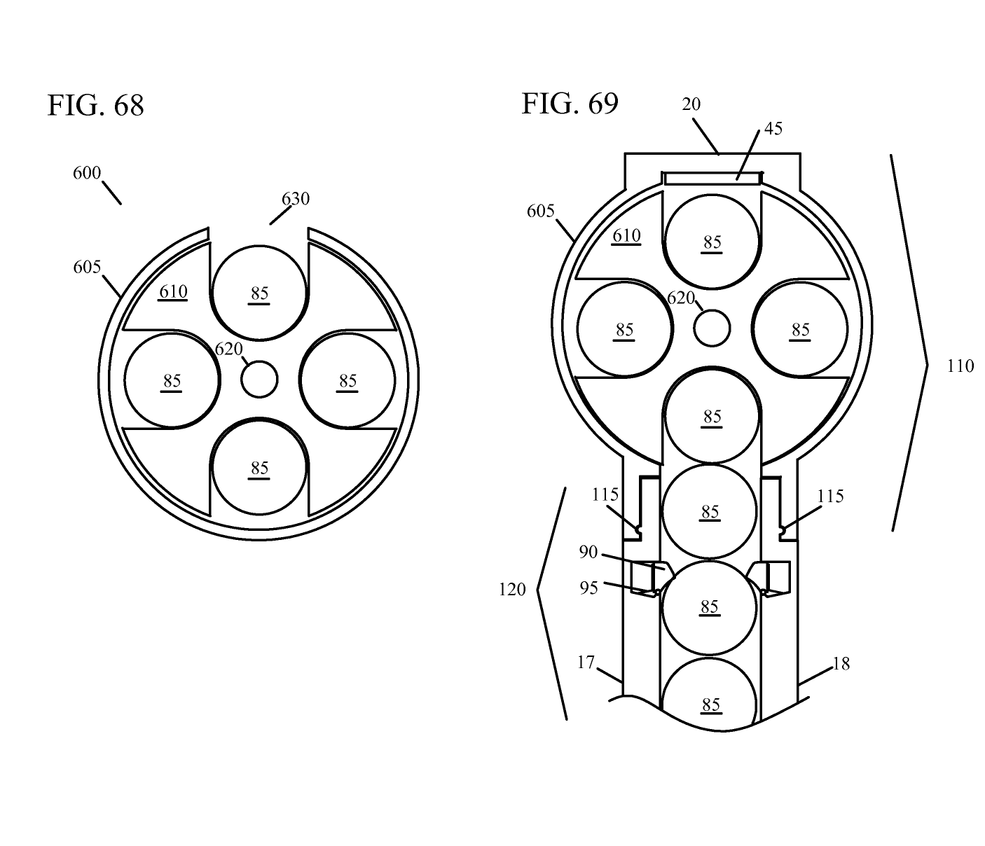

In another embodiment, shown in FIG. 68, a dispenser 600 has a cylindrical housing 605 enclosing a dispensing cylinder 610 which is free to rotate about an axle 620. The cylinder 610 has chambers formed in it in which testing supplies 66, 85, for example lancets 85 in FIG. 68, may be placed. An actuator knob (not shown in this view) connected to the axle 620, and placed external to the housing 605, allows the user to rotate a chamber into alignment with a port 630, through which the testing supply 66, 85 in one chamber may pass.

In an alternate embodiment, shown in FIG. 69, the cylindrical housing 605 may additionally be configured with an ejector and actuator for moving the testing supply 66, 85 through a port in the front of the dispenser. In this example, a slide 45 that is connected to an ejector and grip as described previously in FIGS. 1-8 is used. Optionally, the cylindrical housing 605 and dispensing cylinder may also be used to form a head group 110 for attachment to a magazine group 120.

The dispensers 600 shown in FIGS. 68-69 may be constructed as a single use, disposable dispenser, or a reusable, refillable dispenser. In single use embodiments, the tamper-resistant ampules 100 shown in FIGS. 17-21 may be incorporated at various locations within the dispenser head group 110 and magazine group 120 constructions to mark and/or disable testing supplies if attempts to open the dispenser 600 are made. Further, in some embodiments, the marking reservoir 70 and applicator 75 of FIG. 6 may also, or alternatively, be incorporated to prevent intentional emptying of the dispenser 290 and unauthorized resale of its contents.

Used testing supplies 66, 85 present a biohazard after they have been exposed to blood and must be disposed of properly. Many patients test at various times during the day when they are at work or other locations where it would be inappropriate to dispose of their testing supplies 66, 85 in public waste bins, or other locations where they do not have access to a biohazard receptacle. Therefore, it is advantageous and convenient to add an integrated contaminated testing supply receptacle to testing supply dispensers.

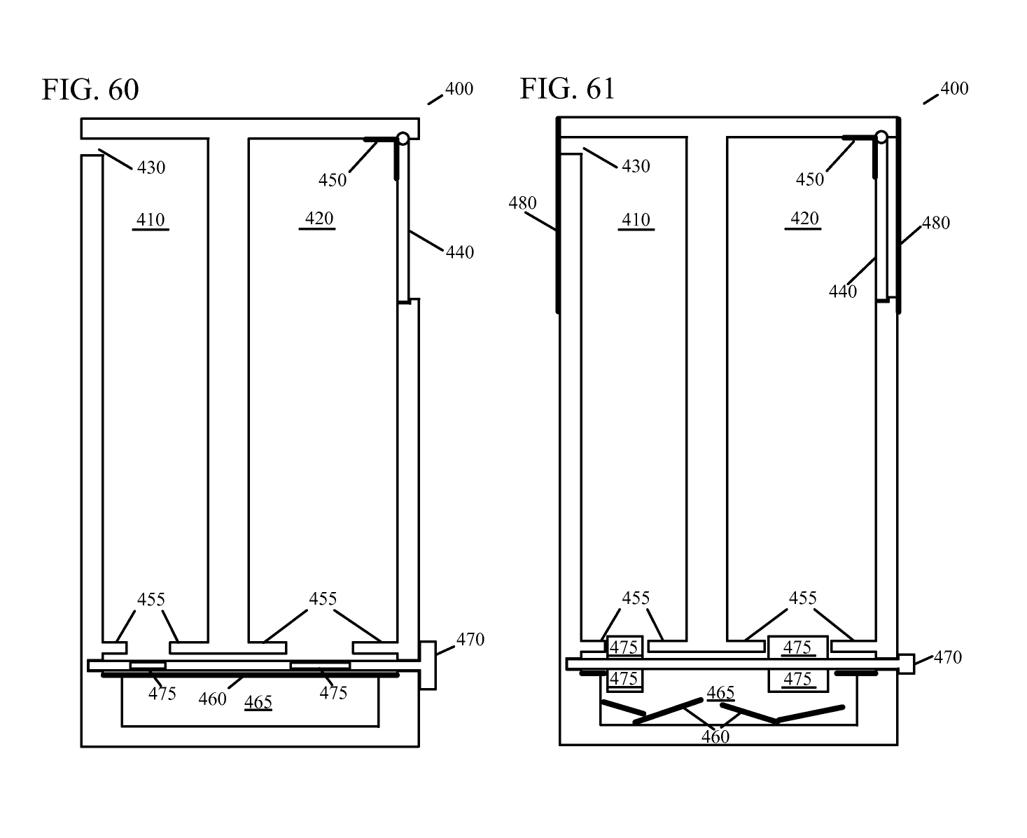

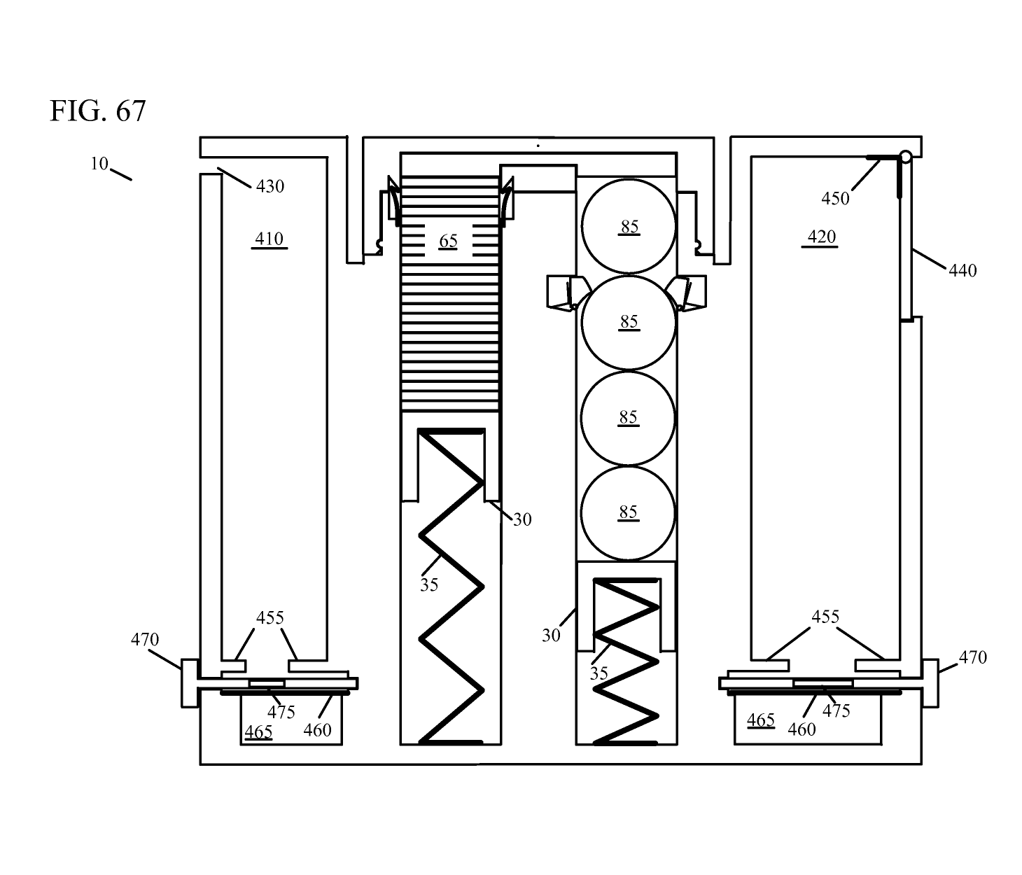

FIGS. 60-61 show an embodiment of a biohazard receptacle 400 that may be attached, either permanently or removably for replacement after filling, to any of the dispenser 10, 290, 600 embodiments described herein. FIG. 60 shows a biohazard receptacle 400 that has two storage volumes 410, 420 for used test strips 66 and used lancets 85, respectively. FIG. 67 shows an embodiment of a dispenser 10 with receptacles 410, 420 for disposal of test strips 66 and lancets 85, respectively. While these examples illustrate a biohazard receptacle 400 for both test strips 66 and lancets 85, the elements shown herein may also be separated to create individual biohazard receptacles 400 for each.

The volume 410 intended for test strip 66 storage has a slot 430 though which used test strips 66 may be inserted. The volume 420 intended for used lancet 85 storage has a spring 450 loaded door 440 that prevents the lancets from exiting the biohazard receptacle 400 after insertion. These volumes 410, 420 may be dimensioned to accommodate any number of used testing supplies. Particularly in the case of disposable dispensers, the volumes 410, 420 are preferrably dimensioned to hold at least as many testing supplies 66, 85 as are contained in the dispenser 10, 290, 600 the biohazard receptacle 400 is associated with.

A sterilizing ampule 465 is created in the bottom of the biohazard receptacle 400, and separated from the storage volumes by a tearable membrane or an easily shattered thin plastic sheet 460 which does not exhibit plastic deformation before shattering. An actuator key 470 is inserted through the biohazard receptacles 400 with wings 475 oriented parallel to the plastic sheet 460 sealing the sterilizing ampule 465. Slotted or perforated dividers 455 separate the key from the storage volumes 410, 420 so the used testing supplies contained in those volumes 410, 420 do not interfere with the operation of the key 470.

When the biohazard receptacle 400 is full and ready for replacement or disposal, a biohazard tape 480 is applied to seal the slot 430 and spring 450 loaded door 440. This biohazard tape 480 may be supplied as a separate peel-and-stick strip, or may already be applied to the biohazard receptacle 400 at another location, and only need be removed and relocated. Once the biohazard receptacle 400 has been sealed, the key 470 is turned at least 90 degrees. This motion causes the wings 470 to tear the membrane or shatter the plastic sheet 460 sealing the sterilizing ampule 465. Sterilizing fluid, such as alcohol or other appropriate antiseptic fluid, is thus released from the sterilizing ampule 465 and is free to flood the sealed storage volumes 410, 420. The sterilizing fluid may be thoroughly distributed by vigorous shaking of the biohazard receptacle 400.

The embodiment of the biohazard receptacle 400 shown in FIGS. 60-61 have the slot 430 spring 450 loaded door 440 on the sides of the slot 430 spring 450 loaded door 440. However, these elements may also or alternatively be located elsewhere on the biohazard receptacle 400, for example, on the top, front, back, or bottom.

Similarly, the sterilizing ampule 465, key 470, and or perforated dividers 455 may be located freely in the biohazard receptacle 400, provided their operation is coordinated with the operation of the other elements of the biohazard receptacle 400.

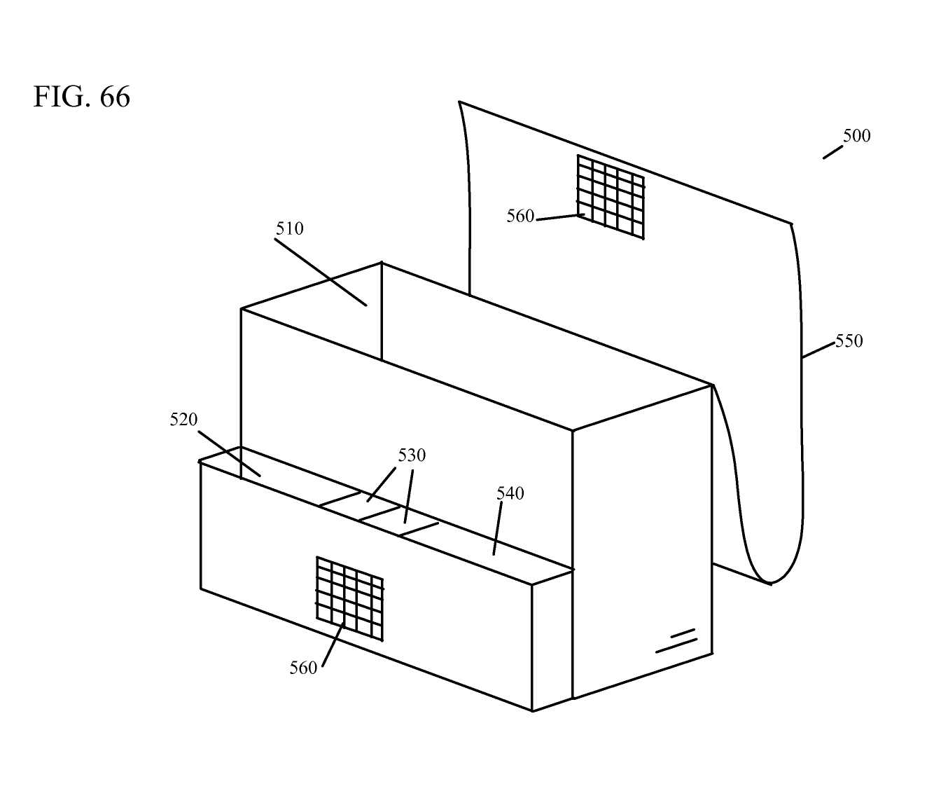

To maintain privacy, add convenience during travel, and coordinate all testing supplies into a single kit, a case may be constructed that contains not only the dispenser 10, 290, 600 but also provides storage space for a blood glucose meter, and at least one pre-filled multi-dose injector pen. An embodiment of such a case 500 is shown in FIG. 66. This embodiment provides a dispenser 10, 290, 600 storage volume 510, a blood glucose meter storage volume 520, two insulin injector pen storage volumes 530, and an additional storage volume 540 that may be used to store additional items such as test supply bulk loaders 200. A cover flap 550, which is preferably constructed of a soft flexible material, is also preferably included. A hook and loop type closure 560, or similar closure, is also preferably provided. The case 500 may also be constructed as a form-fitting cover for the dispenser 10, 290, 600 and made from an elastic material such as rubber, neoprene, or similar materials. In this embodiment, the case 500 also includes testing meter specific pouches.

As a further measure to discourage fraudulent resale of testing supplies, as shown for example in FIG. 1, any of the dispenser embodiments described herein may be constructed with indicia 5 in form of permanent markings applied, for example, through engraving, embossing, pigments included in construction materials, or embedding of labels in transparent construction materials. Indicia 5 include, but are not limited to, human readable indicia related to lot number for tracking purposes, and warnings such as “RESALE PROHIBITED”. Similarly, testing supplies may have computer readable indicia and human readable indicia applied to one of their surfaces that allow tracking of the source of the supplies, and warnings such as “NOT FOR RESALE”. Alternatively, or additionally, computer readable indicia and human readable indicia 5 may include regional specific distribution source codes, and/or an identifier of the reimbursement provider.

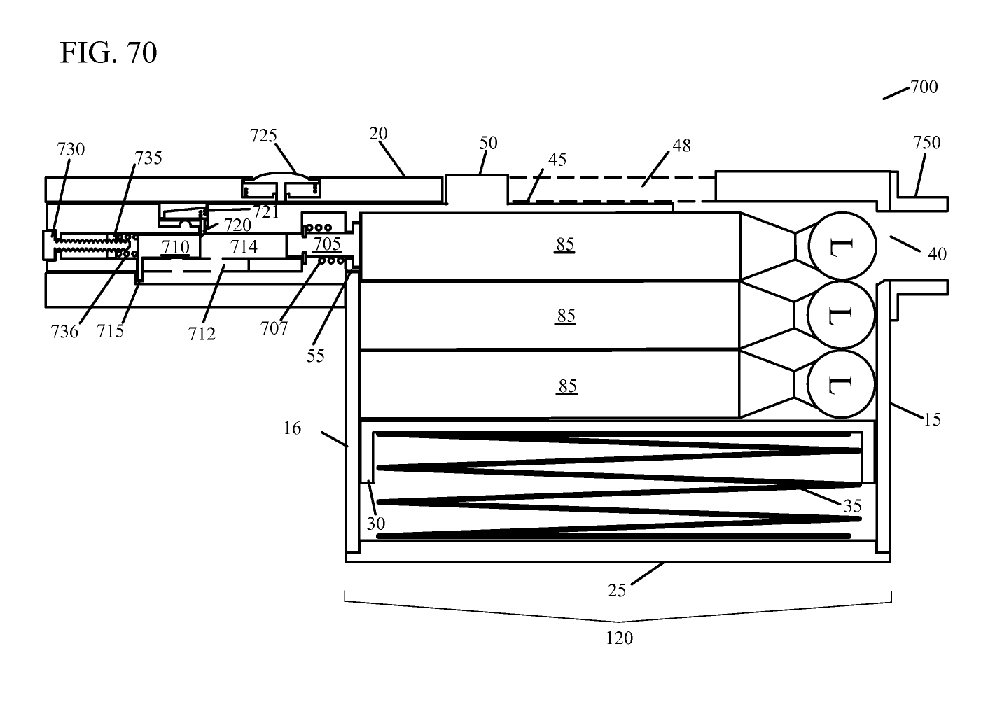

FIGS. 70-75 show a dispenser 700 having an integrated lancing device that is fed by a magazine group 120 of any type as described herein. A slide 45 is located in the dispenser 700 top 20 and is extended with a central channel 714 at its rear. The central channel 714 and slide 45 carry a bolt 710, a bolt release 720, a set screw 730, a set plate 735, a bolt spring 736, and, in combination with the ejector 55, a striker 705 and a striker return spring 707. The top 20 of the dispenser 700 includes the trigger 725.

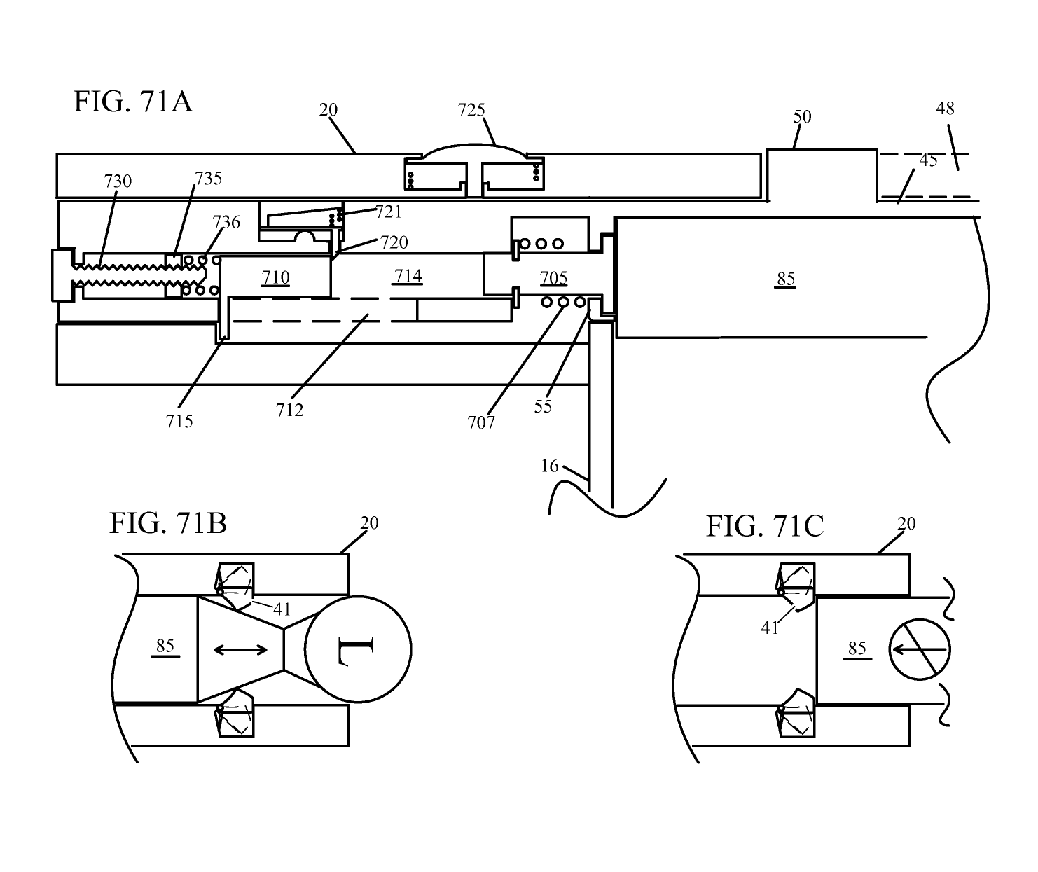

The central channel 714 is constructed with a slot 712 through which the retractor 715 attached to the bolt 710 projects below the bottom of the slide 45. FIG. 70 illustrates the dispenser 700 in a charged and cocked position, with a lancet 85 in a top-most dispensing position, and the bolt 710 held fully retracted by the bolt release 720 against the bias of the bolt spring 736. FIG. 71A shows the elements of the integrated lancing device in greater detail, and also shows the bolt release 720 spring 721.

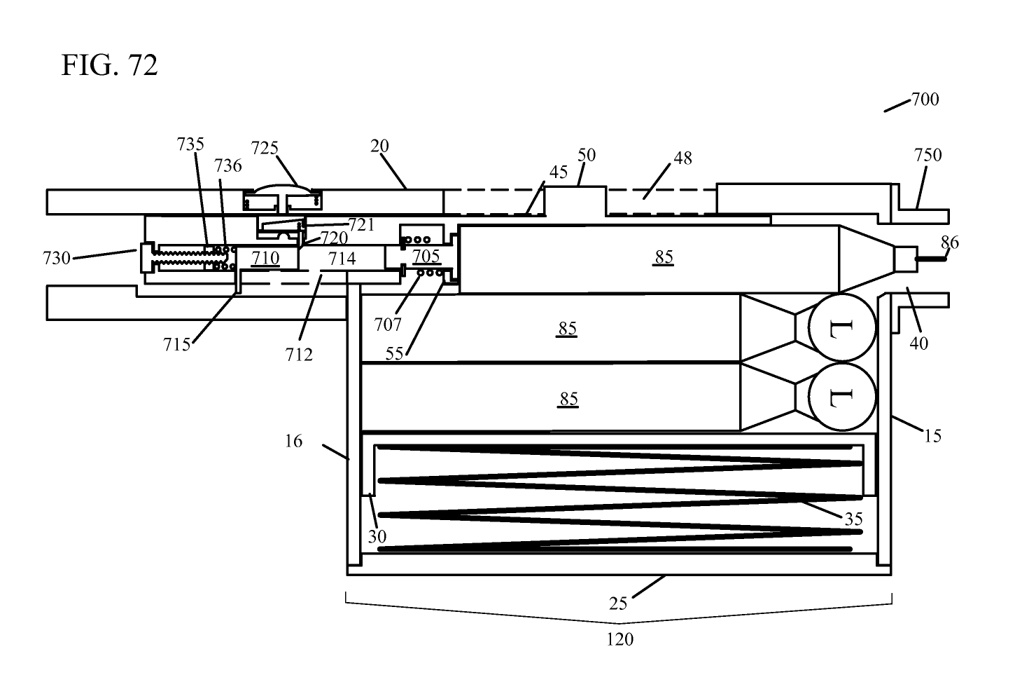

FIG. 72 shows the dispenser 700 in a firing position and the lancet 85 prepared for use with its needle tip 86 exposed. The shroud 750 is removably attached to provide access to the needle tip 86 protective cover, and later removal of the used lancet 85. The position of the lancet 85 in the dispenser 700 is maintained by port 40 regulators 41, shown in FIGS. 71B-71C, which are of similar design and construction to the feed regulators 90 shown in FIGS. 9 and 10, and are located on the left and right inner sides of the dispenser 700 near the port 40. Spring action of the port 40 regulators 41 biases the lancet 85 toward the rear of the dispenser 700, as shown by the double ended arrow in FIG. 71B. To the rear of the lancet, the ejector 55 holds the lancet 85 against the spring bias of the port 40 regulators 41 at the front of the dispenser 700. The slide 45 is maintained in this position by detents (not shown), and the bolt release 720 is brought in alignment with the trigger 725. The lancet 85 needle 86 is held flush with the shroud 750 so that when the shroud is placed against the skin, the needle tip 86 is also flush with the skin.

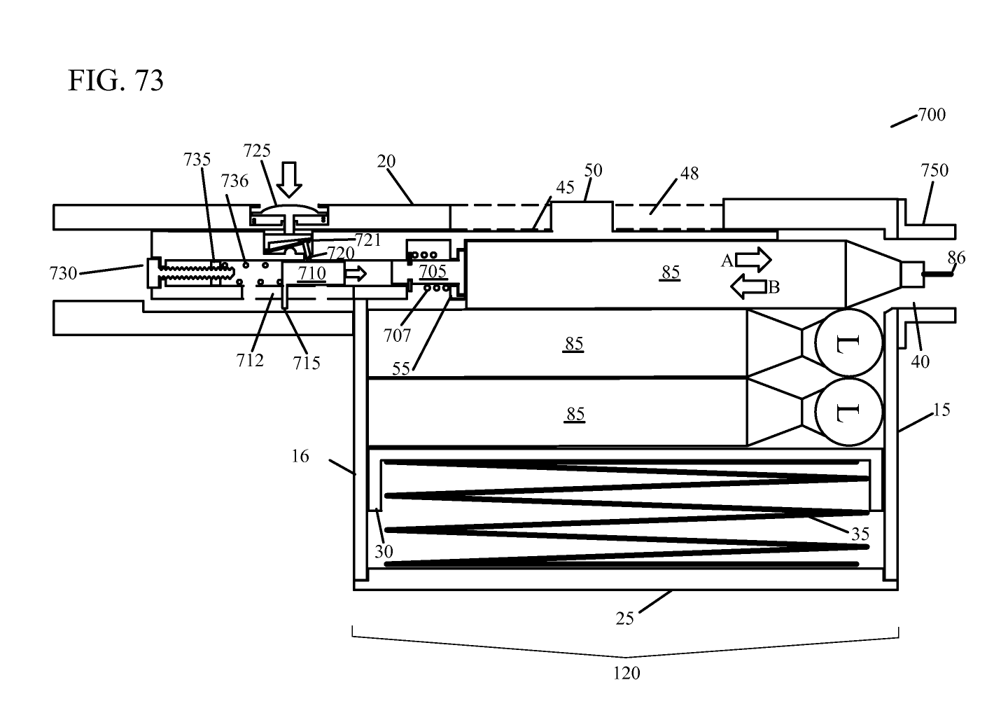

As shown in FIG. 73, actuating the trigger 725 allows the bolt release 720 to disengage the bolt 710 against the bias of the bolt release 720 spring 721. The bolt 710 then accelerates forward, toward the striker 705, under the bias of the bolt spring 736. The bolt 710 then impacts the striker 705, causing the lancet 85 to move forward (arrow A in FIG. 73) against the port 40 regulators 41, which in turn moves the lancet 85 needle tip 86 to extend past the shroud 750 and penetrate the skin. After penetrating the skin, the bias of the port 40 regulators 41 causes the lancet 85 to move backward (see arrow B in FIG. 73, and double ended arrow in FIG. 71B), retracting the needle tip 86 from the skin at the same time.

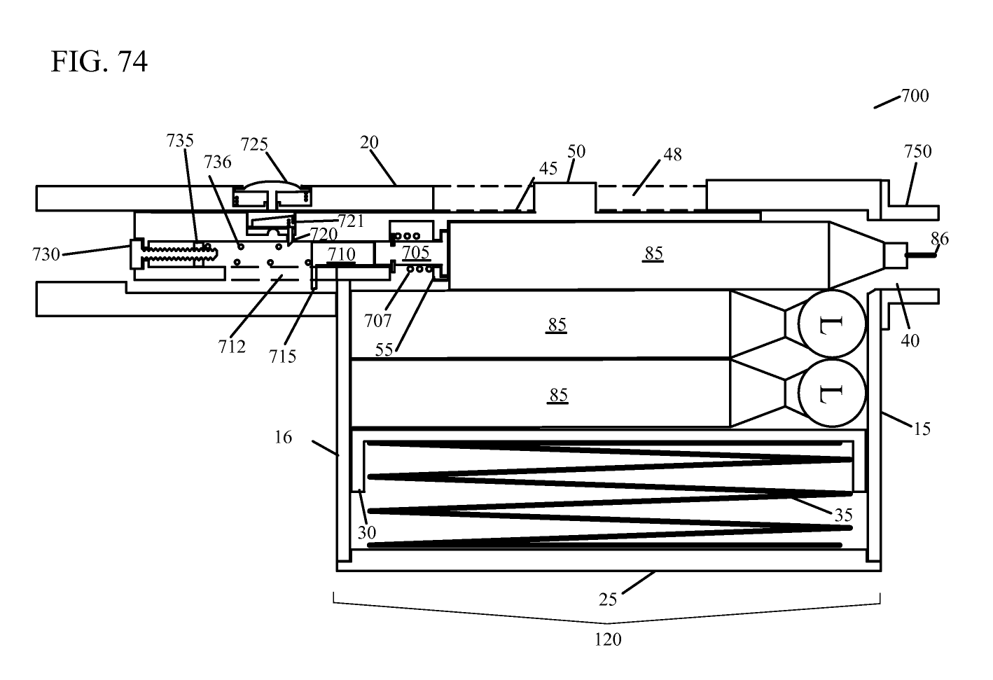

FIG. 74 shows the dispenser 700 after a lancing operation. In this figure, the bolt 710 is full forward against the striker 705. As a result, a second lancing procedure with the same lancet 85 cannot be performed. The bolt 710 must first be reset against the bias of the bolt spring 736 by removing the used lancet 85 and resetting the lancing device assembly.

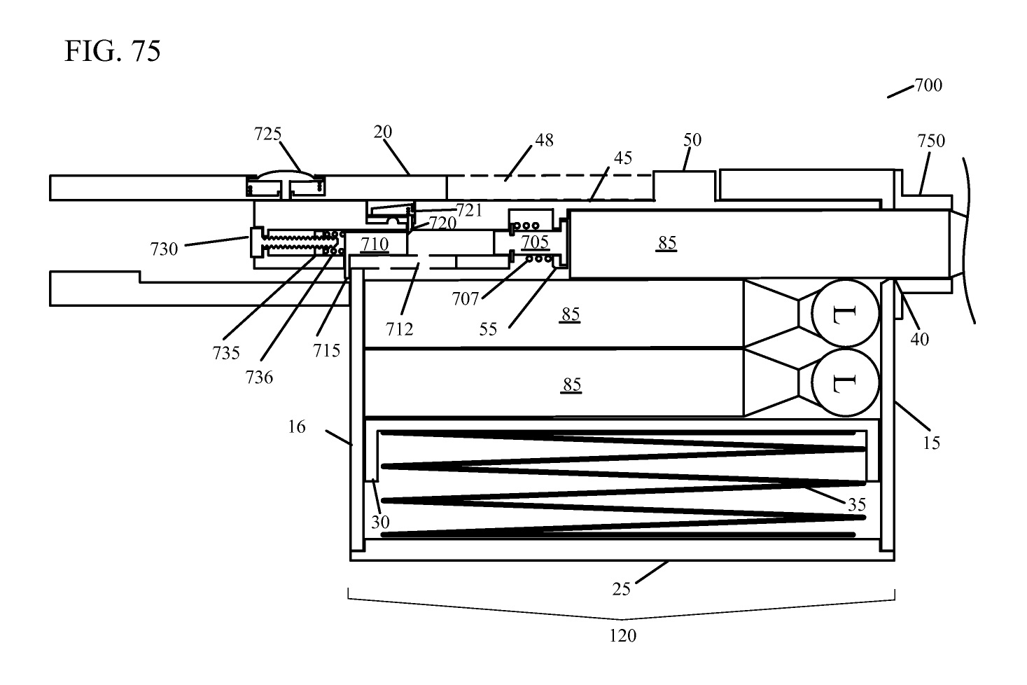

FIG. 75 shows the slide 45 after being moved to its forward-most eject position. The used lancet 85 is pushed past the port 40 regulators 41 (not shown in this view), through the port 40 and the shroud 750. The lancet 85 is now in a position where it can be removed by the patient. During the same operation, the retractor 715 attached to the bolt 710 catches the back 16 of the dispenser 700 and prevents the bolt 710 from further forward movement as the slide 45 moves forward to eject the used lancet 85. As the slide 45 moves forward, the bolt 710 moves past the bolt release 720, which acts as a sear, and is held in its rearmost position against the bias of the now compressed bolt spring 736. The dispenser 700 may now be recharged and prepared for another lancing operation with a fresh lancet by moving the slide 45 back to its rearmost cocked position. As shown in FIG. 71C, once a used lancet 85 has been removed from the dispenser port 40, the shape of the port 40 regulators 41 prevents a used lancet 85 from simply being reinserted after the bolt 710 has been reset.

When the slide 45 is moved fully to the rear cocked position, a new lancet 85 is delivered to the slide from the magazine by the magazine spring 35 and follower 30, as shown in FIGS. 8-10, bringing the dispenser into the same configuration as shown in FIG. 70.

The patient may adjust the lancing depth to suit their own personal comfort level by adjusting the set screw 730. When the set screw 730 is turned, the set plate 735 moves closer to, or farther from, the rear of the bolt 710. This change in distance results in a change in the bolt spring 736 bias, and therefore the amount of force with which the bolt 710 will impact the striker 705.

The set plate 735 is prevented from rotating during adjustment through tabs or other radial extensions that slidably mate to longitudinal grooves or similar surface features on the inside of the central channel 714, allowing the set plate 735 to slide within the central channel 714, but not rotate. Additionally, view windows 150 and graduations 155, similar to those shown in FIG. 51, may be added to the dispenser 700 to allow the patient to see the location of the set plate 735, and therefore verify the lancing depth they have chosen.

The dispenser 700 may be constructed for single use, or for refillable embodiments, by separating the dispenser 700 into a head group 110 (including the dispenser top 20 and various lancing device elements described herein) and a magazine group 120. In single use embodiments, the tamper-resistant ampules 100 shown in FIGS. 17-21 may be incorporated at various locations within the dispenser 700 head group 110 and magazine group 120 constructions to mark and/or disable testing supplies if attempts to open the dispenser 700 are made. Further, in some embodiments, the marking reservoir 70 and applicator 75 of FIG. 6 may also, or alternatively, be incorporated to prevent intentional emptying of the dispenser 700 and unauthorized resale of its contents.

Accordingly, it is to be understood that the embodiments of the invention herein described are merely illustrative of the application of the principles of the invention. Reference herein to details of the illustrated embodiments is not intended to limit the scope of the claims, which themselves recite those features regarded as essential to the invention.

What is claimed is:

1. A diabetic testing supply dispenser comprising:
a) a magazine group having a body with four internal walls, a bottom, and an open top forming a magazine storage volume and a feed pathway for testing supplies in a length between the open top and the bottom, comprising a follower disposed in the magazine storage volume and free to slide within the magazine storage volume for at least a portion of the feed pathway length, and a magazine spring disposed within the magazine storage volume between the follower and the bottom for biasing the follower toward the open top of the magazine storage volume;
b) a head group coupled to the magazine group, comprising a top completely enclosing the open top of the magazine storage volume;
c) an actuator comprising a portion outside the head group for operation by a user and an ejector portion inside the head group for moving a top-most testing supply;
d) a dispenser port in communication with the magazine storage volume, aligned with the ejector portion of the actuator; and
e) at least one feed regulator disposed in the magazine storage volume, moveable to a first default position in the feed pathway and a second position out of the feed pathway, wherein the feed regulator has a first state wherein motion of the testing supplies is only permitted in one direction into the magazine storage volume and a second state wherein motion of the testing supplies is permitted in two opposite directions into and out of the magazine storage volume;
such that, the force of the magazine spring against the follower forces the plurality of testing supplies in the magazine storage volume toward the head group, bringing the top-most testing supply into a position aligned with the dispenser port, and operation of the actuator moves the top-most testing supply out of the dispenser through the dispenser port a sufficient distance that at least a portion of the testing supply may be grasped and the testing supply removed from the dispenser port.

2. The diabetic testing supply dispenser of claim 1, wherein the diabetic testing supply is selected from the group consisting of: a) a test strip, and b) a lancet.

3. The diabetic testing supply dispenser of claim 1, wherein the dispenser port passes through an internal wall of the magazine group.

4. The diabetic testing supply dispenser of claim 1, wherein the head group further comprises at least one internal wall aligned with at least one magazine group internal wall, and the dispenser port passes through the at least one head group internal wall.

5. The diabetic testing supply dispenser of claim 1, wherein the head group and magazine group are permanently coupled.

6. The diabetic testing supply dispenser of claim 1, wherein the actuator is mounted for slideable motion through a slot in the head group from a position opposed to the dispenser port to a position near the dispenser port, and the ejector portion is a flange extending at right angles from the slot, such that when the actuator is slidably moved in the slot from the position opposed to the dispenser port to the position near the dispenser port, a single testing supply is slid through the dispenser port.

7. The diabetic testing supply dispenser of claim 1, wherein the actuator further comprises a thumb wheel outside the head group coupled to a substantially cylindrical roller inside the head group in contact with the top-most testing supply, arranged such that when the thumb wheel is rotated the roller moves the top-most testing supply through the dispenser port.

8. The diabetic testing supply dispenser of claim 1, wherein the actuator further comprises a knob outside the head group coupled to a substantially cylindrical drum inside the head group, the drum having a plurality of drum receptacles for carrying testing supplies, arranged such that, when the knob is rotated, the cylindrical drum moves a first drum receptacle into alignment with the dispenser port.

9. The diabetic testing supply dispenser of claim 8, wherein rotation of the knob further causes a second drum receptacle to move into alignment with the top-most testing supply in the magazine storage volume.

10. The diabetic testing supply dispenser of claim 1, wherein the head group further comprises a reservoir filled with a fluid, connected to an applicator adjacent to the dispenser port, such that as the top-most testing supply passes through the dispensing port the fluid is applied to at least a portion of the testing supply.

11. The diabetic testing supply dispenser of claim 10, wherein the fluid causes a change in color after being applied to a testing supply.

12. The diabetic testing supply dispenser of claim 1, further comprising at least one ampule filled with a fluid and located within a wall of the dispenser, such that if the wall of the dispenser is deformed, the ampule will break and release the fluid into the magazine storage volume.

13. The diabetic testing supply dispenser of claim 12, wherein the fluid is selected from the group consisting of: a) a protein solution, b) a permanent dye, c) an ascorbic acid solution, and d) any combination of a), b), and c).

14. The diabetic testing supply dispenser of claim 1, wherein the magazine group internal walls constrain the testing supplies in the magazine storage volume to form a stack.

15. The diabetic testing supply dispenser of claim 14, wherein the stack is staggered.

16. The diabetic testing supply dispenser of claim 14, wherein the stack is serpentine.

17. The diabetic testing supply dispenser of claim 14, wherein the stack is circular.

18. The diabetic testing supply dispenser of claim 1, wherein, when the head group and magazine group are uncoupled, the at least one feed regulator moves to the position out of the feed pathway when a user pushes a plurality of testing supplies through the open top into the magazine storage volume, and the plurality of testing supplies is prevented from moving back toward the open top by the at least one feed regulator; and
wherein, when the head group and magazine group are coupled, the at least one feed regulator moves to the position out of the feed pathway.

19. The diabetic testing supply dispenser of claim 1, wherein the at least one feed regulator comprises:
a) a barbed sear on a magazine group internal wall proximal to the magazine group open top extending into the magazine storage volume; and
b) a sear block in the head group;
wherein, when a plurality of testing supplies is inserted into the magazine volume through the open top, the barbed sear prevents the magazine spring and follower from pushing the plurality of testing supplies out the open top of the magazine group; and, upon coupling the head group with the magazine group, the sear block retracts the barbed sear from the magazine storage volume, allowing the magazine spring and the follower to move the plurality of testing supplies toward the head group.

20. The diabetic testing supply dispenser of claim 1, wherein the at least one feed regulator comprises:
a) a spring-loaded sear extending into the magazine storage volume proximal to the open top of the magazine storage volume; and
b) a feed lock device on a wall of the magazine group, coupled to the spring-loaded sear to lock the spring-loaded sear in a position within the magazine storage volume to prevent passage of testing supplies within the magazine storage volume;
wherein, when the feed lock is in a free position, the spring-loaded sear can move out of the feed pathway, and when the feed lock is in a locked position the spring-loaded sear is held in the feed pathway.

21. The diabetic testing supply dispenser of claim 20, wherein the actuator maintains the sear in a blocking position when the top-most testing supply is being dispensed.

22. The diabetic testing supply dispenser of claim 1, further comprising a slot extending into the magazine storage volume through which a leader attached to a strap wrapped around the plurality of testing supplies may pass, such that the leader may be pulled by a user to remove the strap from the plurality of testing supplies and the dispenser.

23. The diabetic testing supply dispenser of claim 1, further comprising a bulk loader comprising:
a hollow thin walled body with a first end and a second end, a removable closure at the first end of the hollow thin walled body, and a pusher slidably inserted into the second end of hollow thin walled body for pushing a stack of testing supplies located within the hollow thin walled body between the removable closure and the pusher;
wherein, the first end of the hollow thin walled body is sized to allow the first end of hollow thin walled body to be inserted into the magazine group open top, such that when the pusher is moved from the first end of hollow thin walled body toward the second end of hollow thin walled body, the stack of testing supplies is forced into the magazine storage volume against the bias of the magazine spring.

24. The diabetic testing supply dispenser of claim 1, wherein the magazine group further comprises a biohazard storage volume with at least one port in a wall of the magazine group communicating with the biohazard storage volume.

25. The diabetic testing supply dispenser of claim 24, further comprising an ampule containing a sterilizing fluid, wherein the ampule is adjacent to the biohazard storage volume, and an actuator adjacent to the ampule, wherein operating the actuator fractures the ampule, releasing the sterilizing fluid into the biohazard storage volume.

26. A sealed dispenser containing a plurality of testing supplies to be individually dispensed comprising:
a) a body comprising four internal walls, a bottom, and a top, forming a magazine and a magazine storage volume;
b) an actuator comprising a portion outside the body for operation by a user and an ejector portion inside the body for moving a top-most testing supply to be individually dispensed in the magazine storage volume;
c) a dispenser port in communication with the magazine storage volume, aligned with the ejector portion of the actuator; and
d) at least one tamper-resistant element;
wherein, operation of the actuator moves a top-most testing supply to be individually dispensed out of the sealed dispenser through the dispenser port a sufficient distance that at least a portion of the top-most testing supply may be grasped by a user and the top-most testing supply removed from the dispenser port.

27. The sealed dispenser of claim 26, wherein the at least one tamper-resistant element is a reservoir filled with a fluid, connected to an applicator adjacent to the dispenser port, such that, as the top-most testing supply passes through the dispensing port, the fluid is applied to at least a portion of the top-most element.

28. The sealed dispenser of claim 27, wherein the fluid causes a change in color after being applied to a testing supply.

29. The sealed dispenser of claim 26, wherein the at least one tamper-resistant element is an ampule filled with a fluid and located within a wall of the sealed dispenser, such that if the wall of the sealed dispenser is deformed, the ampule breaks and releases the fluid into the magazine storage volume.

30. The sealed dispenser of claim 29, wherein the fluid is selected from the group consisting of: a) a protein solution, b) a permanent dye, c) an ascorbic acid solution, and d) any combination of a), b), and c).

31. The sealed dispenser of claim 26, wherein the testing supplies are selected from the group consisting of: a) test strips, and b) lancets.

32. The sealed dispenser of claim 26, further comprising at least one feed regulator comprising:
a) a spring-loaded sear extending into the magazine storage volume proximal to the top of the magazine storage volume; and
b) a feed lock device on a wall of the magazine, coupled to the spring-loaded sear to lock the spring-loaded sear in a position within the magazine storage volume to prevent passage of testing supplies within the magazine storage volume;
wherein, when the feed lock is in a free position, the spring-loaded sear can move out of the feed pathway, and when the feed lock is in a locked position the spring-loaded sear is held in the feed pathway.

33. The sealed dispenser of claim 32, wherein the ejector portion of the actuator maintains the spring-loaded sear in a blocking position when the top-most testing supply is being dispensed.

34. The sealed dispenser of claim 26, wherein the dispenser further comprises indicia applied to an external surface.

35. The sealed dispenser of claim 34, wherein the indicia are permanently applied to a material forming the sealed dispenser.

36. An auto-loading lancing dispenser comprising:
a) a dispenser top couplable to a magazine group comprising:
i) a lancing device disposed in the dispenser top, the lancing device having a cocked position, a firing position, and an eject position; and
ii) a trigger mechanism disposed in the dispenser top; and
b) a magazine group having a body with four internal walls, a bottom, and an open top forming a magazine storage volume, comprising a follower disposed below a plurality of lancets in the magazine storage volume and free to slide along at least a portion of a feed pathway length, and a magazine spring disposed between the follower and the bottom, biasing the follower toward the open top;
wherein, when the dispenser top and the magazine group are coupled and the lancing device is in the firing position, actuating the trigger mechanism causes the lancet to move out of the dispenser, penetrate a skin surface to a predetermined depth, and retract into the dispenser;
wherein, when the lancing device is in the eject position a lancet moves to a dispenser port for removal and the lancing device is simultaneously cocked; and
wherein, when the lancing device is in the cocked position, a top-most lancet in the magazine storage volume is delivered to a top-most dispensing position of the lancing device and the lancing device is prevented from firing.

37. The auto-loading lancing dispenser of claim 36, wherein the dispenser top further comprises at least one port regulator that holds a lancet inside the dispenser;
wherein, the lancet protrudes from and then retracts into the dispenser port against a port regulator bias in response to firing of the lancing device;
wherein, the port regulator permits passage of the lancet out of the dispenser port when the lancing device is in the eject position; and
wherein, the port regulator prevents a lancet from being inserted into the lancing device through the dispenser port.

38. The auto-loading lancing dispenser of claim 36, wherein the lancing device comprises a slide with an actuator grip outside the dispenser top, a striker, a bolt with a retractor, a bolt spring, a set plate, a set screw, and a bolt release;
wherein moving the actuator grip and slide places the lancing device in the firing position, and the bolt release holds the bolt in the firing position against a bolt spring bias, such that actuating the trigger releases the bolt and the bolt spring impels the bolt toward the striker and the bolt impacting the striker imparts a force on a lancet in the dispenser;
wherein the lancing device is placed in the eject position by moving the slide in a first slide direction, the bolt retractor limits bolt motion in the first slide direction and compresses the bolt spring against the bolt when the slide is moved in the first slide direction, and the bolt is held by the bolt release when the slide is moved to a first slide direction extreme; and
wherein turning the set screw causes the set plate to move between a first set plate position and a second set plate position, changing the bolt spring bias.

39. The auto-loading lancing dispenser of claim 36, wherein the magazine group further comprises at least one feed regulator extending into the magazine storage volume proximal to the open top, wherein the feed regulator is moveable to a first default position in the feed pathway and a second position out of the feed pathway, and wherein the feed regulator has a first state wherein motion of the testing supplies is only permitted in one direction into the magazine and a second state wherein motion of the testing supplies is permitted in two opposite directions into and out of the magazine.

40. The auto-loading lancing dispenser of claim 39, wherein when the dispenser top and magazine group are coupled, the at least one feed regulator moves out of the feed pathway when the lancing device is in the cocked position, and when the lancing device is moved away from the cocked position, the lancing device holds the at least one feed regulator in a position blocking movement of the plurality of lancets in the magazine storage volume.

41. A bulk loader containing a plurality of diabetic testing supplies for a diabetic testing supply dispenser comprising a testing supply magazine group with an open top, the bulk loader comprising:
a) a hollow body with a first end and a second end;
b) a removable closure at the first end of the hollow body; and
c) a movable pusher inserted into the second end of the hollow body;
wherein, the plurality of testing supplies is disposed between the removable closure and the pusher; and
wherein, when the removable closure is removed, the first end of the hollow body is inserted into the magazine group open top, and the pusher is moved from the second end of the hollow body to the first end of the hollow body, the plurality of testing supplies is pushed into the open top of the magazine group.

42. The bulk loader of claim 41, wherein the testing supply magazine group of the testing supply dispenser further comprises a body with four internal walls and a bottom, forming a magazine storage volume and a feed pathway for testing supplies in a length between the open top and the bottom, and comprises a follower disposed in the magazine storage volume and free to slide within the magazine storage volume for at least a portion of the feed pathway length, and a magazine spring disposed within the magazine storage volume between the follower and the bottom for biasing the follower toward the open top of the magazine storage volume.
